(19) United States
(12) Patent Application Publication
(10) Pub. No.: US 2025/0032576 A1
(43) Pub. Date: Jan. 30, 2025

(54) CNP Therapy

(71) Applicant: BIOMARIN PHARMACEUTICAL INC., Novato, CA (US)

(72) Inventors: George Jeha, Novato, CA (US); Christopher Bauer, Oakland, CA (US); Sergio Covarrubias, Novato, CA (US); Devanshi Shanghavi, Novato, CA (US); Yu-Shan Tseng, Novato, CA (US); Jonathan Day, Buckinghamshire (GB); Elena Fisheleva, Novato, CA (US)

(21) Appl. No.: 18/717,353

(22) PCT Filed: Dec. 7, 2022
(86) PCT No.: PCT/US22/81094

§ 371 Date: Jun. 6, 2024

Related U.S. Application Data
(60) Provisional application No. 63/380,509, filed on Oct. 21, 2022.
(60) Provisional application No. 63/286,829, filed on Dec. 7, 2021.

(51) Int. Cl. A61K 38/17; A61P 19/08; C07K 14/435; G01N 33/573; G01N 33/68

(52) U.S. Cl. CPC A61K 38/17; A61P 19/08; C07K 14/435; G01N 33/573; G01N 33/6893; G01N 2800/52

(57) ABSTRACT

The present disclosure relates, in general, to measures of efficacy in patients receiving C-type natriuretic peptide (CNP) therapy to treat a skeletal dysplasia, short stature or bone-related disorders.

### CROSS-REFERENCE TO RELATED APPLICATIONS

The present application claims the priority benefit of U.S. Provisional Patent Application No. 63/286,829, filed Dec. 7, 2021, and U.S. Provisional Patent Application No. 63/380,509, filed Oct. 21, 2022, herein incorporated by reference in their entireties.

### REFERENCE TO THE SEQUENCE LISTING

This application includes a sequence listing submitted electronically, in a file entitled: 56861_Seqlisting.xml created on Nov. 30, 2022 and having a size of 55,905 bytes, which is incorporated by reference herein.

### FIELD OF THE DISCLOSURE

The present disclosure relates, in general, to c-type natriuretic (CNP) therapy to treat skeletal dysplasia, and measures of efficacy in treatment.

### BACKGROUND

C-type Natriuretic Peptide (CNP) is a paracrine growth factor widely expressed across numerous tissues (Prickett et al., Peptides 2020; 132:170363) with diverse functions including regulation of endochondral bone growth, blood flow and pressure in the microcirculation, anti-inflammatory actions, gamete maturation and neurogenesis and connectivity (Kuhn M., Physiol Rev 2016; 96:751-804). The best defined of these factors in humans is the crucial role of the hormone in skeletal growth in driving growth plate expansion.

Studies in experimental animals show that it is the local production of CNP acting via its specific receptor NPR2 within growth plate tissues that determines physiological endochondral bone growth (Nakao et al., Sci Rep 2015; 5:10554). Study of the dynamic role of CNP in the growth of children is challenging due to the rapid clearance of CNP and very low concentrations in plasma. However, an inactive portion of the synthesized product in tissues (proCNP)—amino terminal proCNP (NTproCNP)—is not subject to clearance or rapid degradation. Its level in plasma reflects variations in linear growth velocity throughout growth in both children and experimental animals (Espiner et al., Horm Res Paediatr 2018; 90:345-357). Notably in subjects with genetic disorders of skeletal growth affecting CNP pathway activity plasma NTproCNP concentrations are raised where intra cellular CNP pathway activity is reduced (Olney et al., J Clin Endocrinol Metab 2015; 100: E355-359; Wang et al., Hum Mutat 2015; 36:474-481) and are reduced where intra cellular activity is enhanced (Hannema et al., J Clin Endocrin Metab 2013; 98: E1988-1998; Boudin et al., Am J Hum Genet 2018; 103:288-295; Miura et al. PloS one 2012; 7: e42180). In achondroplasia, the normal reciprocal antagonism (Ozasa et al., Bone 2005 36:1056-1064) between FGFR3 pathway activity (inhibitory to endochondral bone growth) and CNP signaling (stimulating bone growth) is overridden by a gain of function mutation in FGR3 (Yasoda et al. Nature Medicine 2004 10:80-86), reducing intracellular CNP activity, and is associated with modest elevations in concentrations of CNP products in plasma (Olney et al., J Clin Endocrinol Metab 2015 100: E355-359).

Nothing is known of the dynamics or significance of feedback regulation of CNP during periods of active long bone growth. Further, it is unclear whether such feedback is direct or time-dependent on inter-cellular growth responses of skeletal tissues. (indirect feedback). Direct feedback results from actions of the cell's own product on CNP production whereas indirect feedback involves a longer loop mediated by cells other than those secreting the peptide. However in a recent report addressing these important issues in rodent pups, exogenous CNP administered at high concentrations continuously for 3 days inhibited CNP gene expression but only in tissues containing growth plates (Ueda et al., PLOS One 2020, 15: e0240023).

### SUMMARY

The present disclosure is directed to observations in real time on the impact of exogenous CNP analogue (e.g., vosoritide) on endogenous CNP production in children with achondroplasia (Ach) during a 5-year period of daily treatment. Analysis of endogenous CNP levels in response to CNP treatment vary depending on the dose of exogenous CNP given and the state of growth of the subject. The present disclosure shows that levels of NTproCNP (indicative of endogenous CNP levels) and N-terminal fragment of collagen X (CXM) are useful as markers of growth velocity and efficacy of endogenous CNP therapy in children with short stature or skeletal dysplasia, such as achondroplasia. Additional measures of efficacy in younger children include analysis of skull and brain morphology over time.

Provided herein is a method of treating a subject having a bone-related disorder, skeletal dysplasia or short stature and receiving C-type natriuretic peptide (CNP) therapy, comprising i) administering CNP therapy to the subject; ii) obtaining a sample from the subject; iii) measuring levels of NTproCNP and/or N terminal fragment of collagen X (CXM) in a sample collected from the subject in (ii); and iv) altering or changing the dose of CNP to bring NTproCNP levels within +/−2 SDS of mean NTproCNP for the population.

In various embodiments, CNP therapy dose level or frequency increases if the level of NTproCNP increases, or CNP therapy dose level decreases if the level of NTproCNP decreases.

Also provided is a method of treating a subject having a bone-related disorder, skeletal dysplasia or short stature and receiving C-type natriuretic peptide (CNP) therapy, comprising i) administering CNP therapy to the subject; ii) obtaining a sample from the subject; iii) measuring levels of N terminal fragment of collagen X (CXM) in a sample collected from the subject in (ii); and, iv) increasing CNP therapy dose level or frequency if the level of collagen X decreases.

In various embodiments, increasing the CNP therapy dose increases the average growth velocity (AGV) in the subject. In various embodiments, the average growth velocity (AGV) in the subject increases over 6 months, over 1 year or over 2 years, or more.

In various embodiments, increasing CNP therapy dose comprises increasing dose frequency or increasing dose amount.

In various embodiments, an increase in CNP therapy dose level and decrease in NTproCNP level correlate with improved Annualized Growth Velocity (AGV) in subjects.

In various embodiments, an increase in CNP therapy dose level and decrease in NTproCNP level extends the duration of growth plate activity in the subject.

In various embodiments, the levels of NTproCNP are maintained between 2 standard deviations of mean NTproCNP levels based on population analysis. In various embodiments, the levels of NTproCNP are maintained between +/−2 SDS of the mean NTproCNP for that population. In various embodiments, the NTproCNP is ±0.5, ±1.0, ±1.5 or ±2.0 standard deviations (SDS) of mean NTproCNP levels of a population to which the subject is grouped.

In various embodiments, the CNP therapy is titrated toward zero NTproCNP SDS if the NTproCNP SDS is below the mean. In various embodiments, the CNP therapy is titrated until zero NTproCNP SDS. In various embodiments, the CNP therapy is titrated until +0.5, +1.0, +1.5 or +2.0 NTproCNP SDS for the population being treated is achieved. In various embodiments, the zero NTproCNP SDS predicts optimal effect size. Optimal size effect is a measure of the expected average normal growth rate of a subject based on population norms.

In various embodiments, the sample is blood, urine, plasma, saliva, or tissue.

In various embodiments, the subject is suffering from a bone-related disorder, skeletal dysplasia or short stature. In various embodiments, the bone-related disorder, skeletal dysplasia or short stature is selected from the group consisting of achondroplasia, osteoarthritis, hypophosphatemic rickets, hypochondroplasia, short stature, dwarfism, osteochondrodysplasias, thanatophoric dysplasia, osteogenesis imperfecta, achondrogenesis, chondrodysplasia punctata, homozygous achondroplasia, camptomelic dysplasia, congenital lethal hypophosphatasia, perinatal lethal type of osteogenesis imperfecta, short-rib polydactyly syndromes, rhizomelic type of chondrodysplasia punctata, Jansen-type metaphyseal dysplasia, spondyloepiphyseal dysplasia congenita, atelosteogenesis, diastrophic dysplasia, congenital short femur, Langer-type mesomelic dysplasia, Nievergelt type mesomelic dysplasia, Robinow syndrome, Reinhardt syndrome, acrodysostosis, peripheral dysostosis, Kniest dysplasia, fibrochondrogenesis, Roberts syndrome, acromesomelic dysplasia, micromelia, Morquio syndrome, Kniest syndrome, metatrophic dysplasia, spondyloepimetaphyseal dysplasia, disorders related to NPR2 mutation, SHOX mutation (Turner's syndrome/Leri Weill), PTPN11 mutations (Noonan's syndrome) and IGF1R mutation.

It is contemplated that CNP therapy to treat a subject having a bone-related disorder, skeletal dysplasia or short stature comprises administration of CNP variants, conjugates, salts or prodrugs thereof.

In various embodiments the CNP variants are useful as an adjunct or alternative to growth hormone for treating idiopathic short stature and other skeletal dysplasias.

In various embodiments, the bone-related disorder, skeletal dysplasia or short stature disorder results from an NPR2 mutation, SHOX mutation (Turner's syndrome/Leri Weill), or PTPN11 mutations (Noonan's syndrome).

In various embodiments, the bone-related disorder, skeletal dysplasia or short stature disorder results from an NPR2 mutation, SHOX mutation (Turner's syndrome/Leri Weill), PTPN11 mutations (Noonan's syndrome), or insulin growth factor 1 receptor (IGF1R).

In various embodiments, the CNP variants are useful to treat growth plate disorders and short stature, including familial short stature, dominant familial short stature which is also known as dominant inherited short stature, or idiopathic short stature. In various embodiments, the short stature or growth plate disorder is a result of a mutation in collagen (COL2A1, COL11A1, COL9A2, COL10), aggrecan (ACAN), indian hedgehog (IHH), PTPN11, NPR2, NPPC, or FGFR3.

In various embodiments, the gene related to skeletal dysplasia or short stature is selected from the group consisting of NPR2, SHOX, PTPN11, COL2A1, COL11A1, COL9A2, COL10), aggrecan (ACAN), indian hedgehog (IHH), NPPC, FGFR3, IGF1R, DTL, and pregnancy-associated plasma protein A2 (PAPPA2), or combinations thereof.

In various embodiments, the growth plate disorder or short stature is associated with one or more mutations in a gene associated with a RASopathy.

In various embodiments, the bone-related disorder, skeletal dysplasia or short stature disorder results from a RASopathy. In various embodiments, the RASopathy is Noonan syndrome, Costello syndrome, cardiofaciocutaneous syndrome, neurofibromatosis Type 1, or LEOPARD syndrome. In one embodiment, the RASopathy is hereditary gingival fibromatosis type 1.

In various embodiments, the CNP variants are useful to treat a subject with short stature having a height SDS of less than −1.0, −1.5, −2.0, −2.5, or −3.0, and having at least one parent with a height SDS of less than −1.0, −1.5, −2.0 or −2.5, optionally wherein the second parent has height within the normal range. In various embodiments, the CNP variants are useful to treat a subject with short stature having a height SDS of between −2.0 to −3.0. In various embodiments, the CNP variants are useful to treat a subject with short stature having a height SDS of between −2.0 to −2.5. In various embodiments, the short stature is associated with one or more mutations in a gene associated with short stature, such as, collagen (COL2A1, COL11A1, COL9A2, COL10), aggrecan (ACAN), indian hedgehog (IHH), PTPN11, NPR2, NPPC, FGFR3, or insulin growth factor 1 receptor (IGF1R), or combinations thereof. In various embodiments, the gene related to skeletal dysplasia or short stature is selected from the group consisting of NPR2, SHOX, PTPN11, COL2A1, COL11A1, COL9A2, COL10), aggrecan (ACAN), indian hedgehog (IHH), NPPC, FGFR3, IGF1R, DTL, and pregnancy-associated plasma protein A2 (PAPPA2) or combinations thereof. In various embodiments, the growth plate disorder or short stature is associated with one or more mutations in a gene associated with a RASopathy.

In various embodiments, the short stature is a result of mutations in multiple genes as determined by polygenic risk score (PRS). In various embodiments, the subject has a mutation in NPR2 and a low PRS. In various embodiments, the subject has a mutation in FGFR3 and a low PRS. In various embodiments, the subject has a mutation in NPR2 and a low PRS. In various embodiments, the subject has a mutation in IGF1R and a low PRS. In various embodiments, the subject has a mutation in NPPC and a low PRS. In various embodiments, the subject has a mutation in SHOX and a low PRS. In various embodiments, the subject has one or more mutation in one or more of FGFR3, IGF1R, NPPC, NPR2 and SHOX, and a low PRS. In various embodiments, the PRS is 1 or 2. In various embodiments, the PRS is 1. In various embodiments, the PRS is 2.

In various embodiments, the CNP is a CNP variant selected from the group consisting

(Pro-Gly-CNP37)
(SEQ ID NO: 1)
of PGQEHPNARKYKGANKKGLSKGCFGLKLDRIGSMSGLGC;
(Gly-CNP-37)
(SEQ ID NO: 2)
GQEHPNARKYKGANKKGLSKGCFGLKLDRIGSMSGLGC;
(Gly-CNP53)
(SEQ ID NO: 3)
GDLRVDTKSRAAWARLLQEHPNARKYKGANKKGLSKGCFGLKLDRIGSM
SGLGC;
(Pro-CNP53)
(SEQ ID NO: 4)
PDLRVDTKSRAAWARLLQEHPNARKYKGANKKGLSKGCFGLKLDRIGSM
SGLGC;
(Met-CNP53)
(SEQ ID NO: 5)
MDLRVDTKSRAAWARLLQEHPNARKYKGANKKGLSKGCFGLKLDRIGSM
SGLGC;
[CNP-53(M48N)]
(SEQ ID NO: 6)
DLRVDTKSRAAWARLLQEHPNARKYKGANKKGLSKGCFGLKLDRIGSNS
GLGC;
(CNP-52)
(SEQ ID NO: 7)
LRVDTKSRAAWARLLQEHPNARKYKGANKKGLSKGCFGLKLDRIGSMSG
LGC;
(CNP-51)
(SEQ ID NO: 8)
RVDTKSRAAWARLLQEHPNARKYKGANKKGLSKGCFGLKLDRIGSMSGL
GC;
(CNP-50)
(SEQ ID NO: 9)
VDTKSRAAWARLLQEHPNARKYKGANKKGLSKGCFGLKLDRIGSMSGLG
C;
(CNP-49)
(SEQ ID NO: 10)
DTKSRAAWARLLQEHPNARKYKGANKKGLSKGCFGLKLDRIGSMSGLG
C;
(CNP-48)
(SEQ ID NO: 11)
TKSRAAWARLLQEHPNARKYKGANKKGLSKGCFGLKLDRIGSMSGLGC;
(CNP-47)
(SEQ ID NO: 12)
KSRAAWARLLQEHPNARKYKGANKKGLSKGCFGLKLDRIGSMSGLGC;
(CNP-46)
(SEQ ID NO: 13)
SRAAWARLLQEHPNARKYKGANKKGLSKGCFGLKLDRIGSMSGLGC;
(CNP-45)
(SEQ ID NO: 14)
RAAWARLLQEHPNARKYKGANKKGLSKGCFGLKLDRIGSMSGLGC;
(CNP-44)
(SEQ ID NO: 15)
AAWARLLQEHPNARKYKGANKKGLSKGCFGLKLDRIGSMSGLGC;
(CNP-43)
(SEQ ID NO: 16)
AWARLLQEHPNARKYKGANKKGLSKGCFGLKLDRIGSMSGLGC;
(CNP-42)
(SEQ ID NO: 17)
WARLLQEHPNARKYKGANKKGLSKGCFGLKLDRIGSMSGLGC;
(CNP-41)
(SEQ ID NO: 18)
ARLLQEHPNARKYKGANKKGLSKGCFGLKLDRIGSMSGLGC;
(CNP-40)
(SEQ ID NO: 19)
RLLQEHPNARKYKGANKKGLSKGCFGLKLDRIGSMSGLGC;
(CNP-39)
(SEQ ID NO: 20)
LLQEHPNARKYKGANKKGLSKGCFGLKLDRIGSMSGLGC;
(CNP-38)
(SEQ ID NO: 21)
LQEHPNARKYKGANKKGLSKGCFGLKLDRIGSMSGLGC;
(CNP-37)
(SEQ ID NO: 22)
QEHPNARKYKGANKKGLSKGCFGLKLDRIGSMSGLGC;
(CNP-36)
(SEQ ID NO: 23)
EHPNARKYKGANKKGLSKGCFGLKLDRIGSMSGLGC;
(CNP-35)
(SEQ ID NO: 24)
HPNARKYKGANKKGLSKGCFGLKLDRIGSMSGLGC;
(CNP-34)
(SEQ ID NO: 25)
PNARKYKGANKKGLSKGCFGLKLDRIGSMSGLGC;
(CNP-33)
(SEQ ID NO: 26)
NARKYKGANKKGLSKGCFGLKLDRIGSMSGLGC;
(CNP-32)
(SEQ ID NO: 27)
ARKYKGANKKGLSKGCFGLKLDRIGSMSGLGC;
(CNP-31)
(SEQ ID NO: 28)
RKYKGANKKGLSKGCFGLKLDRIGSMSGLGC;
(CNP-30)
(SEQ ID NO: 29)
KYKGANKKGLSKGCFGLKLDRIGSMSGLGC;
(CNP-29)
(SEQ ID NO: 30)
YKGANKKGLSKGCFGLKLDRIGSMSGLGC;
(CNP-28)
(SEQ ID NO: 31)
KGANKKGLSKGCFGLKLDRIGSMSGLGC;
(CNP-27)
(SEQ ID NO: 32)
GANKKGLSKGCFGLKLDRIGSMSGLGC;
(CNP-26)
(SEQ ID NO: 33)
ANKKGLSKGCFGLKLDRIGSMSGLGC;
(CNP-25)
(SEQ ID NO: 34)
NKKGLSKGCFGLKLDRIGSMSGLGC;
(CNP-24)
(SEQ ID NO: 35)
KKGLSKGCFGLKLDRIGSMSGLGC;
(CNP-23)
(SEQ ID NO: 36)
KGLSKGCFGLKLDRIGSMSGLGC;
(CNP-21)
(SEQ ID NO: 37)
LSKGCFGLKLDRIGSMSGLGC;
(CNP-20)
(SEQ ID NO: 38)
SKGCFGLKLDRIGSMSGLGC;
(CNP-19)
(SEQ ID NO: 39)
KGCFGLKLDRIGSMSGLGC;
(CNP-18)
(SEQ ID NO: 40)
GCFGLKLDRIGSMSGLGC;
[CNP-37(M32N)]
(SEQ ID NO: 41)
QEHPNARKYKGANKKGLSKGCFGLKLDRIGSNSGLGC;
(Pro-CNP-37)
(SEQ ID NO: 42)
PQEHPNARKYKGANKKGLSKGCFGLKLDRIGSMSGLGC;
(Met-CNP-37)
(SEQ ID NO: 43)
MQEHPNARKYKGANKKGLSKGCFGLKLDRIGSMSGLGC;
[Gly-CNP-37(M32N)]
(SEQ ID NO: 44)
GQEHPNARKYKGANKKGLSKGCFGLKLDRIGSNSGLGC;
(Met-Gly-CNP-37)
(SEQ ID NO: 45)
MGQEHPNARKYKGANKKGLSKGCFGLKLDRIGSMSGLGC;
(SEQ ID NO: 46)
PGQEHPQARRYRGAQRRGLSRGCFGLKLDRIGSMSGLGC;
(SEQ ID NO: 47)
PGQEHPNARKYKGANKKGLSKGCFGLKLDRIGSMSGLGC;
(SEQ ID NO: 48)
PGQEHPNARRYRGANRRGLSRGCFGLKLDRIGSMSGLGC;
and
(SEQ ID NO: 49)
PGQEHPQARKYKGAQKKGLSKGCFGLKLDRIGSMSGLGC.

In various embodiments, the variant peptide further comprises an acetyl group. In various embodiments, the acetyl group is on the N-terminus of the peptide. In various embodiments, the peptide further comprises an OH or an NH₂ group at the C-terminus.

In various embodiments, the CNP variant composition is an extended release composition. In various embodiments, the composition is a sustained release composition. In various embodiments the sustained or extended release compositions comprises a CNP variant pro-drug.

In various embodiments, the variant peptide comprises a conjugate moiety. In various embodiments, the conjugate moiety is on a residue of the CNP cyclic domain or at a site other than the CNP cyclic domain. In various embodiments, the conjugate moiety is on a lysine residue. In various embodiments, the conjugate moiety comprises one or more acid moieties. In various embodiments, the acid moiety is a hydrophobic acid.

In various embodiments, the conjugate moiety comprises one or more acid moieties linked to a hydrophilic spacer. In various embodiments, the hydrophilic spacer is any amino acid. In various embodiments, the hydrophilic spacer is gamma glutamic acid (γGlu). In various embodiments, the hydrophilic spacer is OEG (8-amino-3,6-dioxaoctanoic acid). In various embodiments, the hydrophilic spacer is gamma glutamic acid (γGlu) or OEG (8-amino-3,6-dioxaoctanoic acid). In various embodiments, the hydrophilic spacer is gamma glutamic acid (γGlu) linked to one or two or more OEG (8-amino-3,6-dioxaoctanoic acid). In various embodiments, the acid moiety is a fatty acid. Exemplary fatty acids include short chain, medium chain, or long chain fatty acids, or a dicarboxylic fatty acid. In various embodiments, the fatty acid is saturated or unsaturated. Contemplated are C-6 to C-20 fatty acids, including but not limited to, C-6, C-8, C-10, C-12, C-14, C-16, C-18 or C-20 fatty acids, saturated or unsaturated. In various embodiments, the fatty acid is decanoic acid, dodecanoic acid, myristic acid, palmitic acid, stearic acid, arachidic acid, or diacids of the same.

In various embodiments, the acid moiety and the hydrophilic spacer have the structure AEEA-AEEA-γGlu-C18DA.

In various embodiments, the variant comprises one or more linker groups. In various embodiments, the linker is on a residue of the CNP cyclic domain or at a site other than the CNP cyclic domain. In various embodiments, the linker is on a lysine residue.

In various embodiments, the linker is a hydrolysable linker.

In various embodiments, the conjugate moiety is a synthetic polymeric group. In various embodiments, the variant comprises a synthetic polymeric group coupled to the variant through a hydrolysable linker. In various embodiments, the synthetic polymeric group comprises a hydrophilic polymer moiety. In various embodiments, the hydrophilic polymer moiety comprises polyethylene glycol (PEG). In various embodiments, the hydrophilic polymer moiety comprises polyethylene glycol (PEG) having a 6 to 20 atom chain length.

In various embodiments, the CNP variant is

(Pro-Gly-CNP37)
(SEQ ID NO: 1)
PGQEHPNARKYKGANKKGLSKGCFGLKLDRIGSMSGLGC;
(Gly-CNP-37)
(SEQ ID NO: 2)
GQEHPNARKYKGANKKGLSKGCFGLKLDRIGSMSGLGC;
or
(CNP-38)
(SEQ ID NO: 21)
LQEHPNARKYKGANKKGLSKGCFGLKLDRIGSMSGLGC.

In various embodiments, the variant has the structure:

(SEQ ID NO: 46)
PGQEHPQARRYRGAQRRGLSRGCFGLK(AEEA-AEEA-γGlu-C18DA)
LDRIGSMSGLGC,
or
(SEQ ID NO: 46)
Ac-PGQEHPQARRYRGAQRRGLSRGCFGLK(AEEA-AEEA-γGlu-
C18DA)LDRIGSMSGLGC-OH.

In various embodiments, the variant is selected from the group consisting of

(SEQ ID NO: 46)
Ac-PGQEHPQARRYRGAQRRGLSRGCFGLKLDRIGSMSGLGC-OH;
(SEQ ID NO: 47)
Ac-PGQEHPNARKYKGANKKGLSKGCFGLKLDRIGSMSGLGC-NH₂;
(SEQ ID NO: 48)
Ac-PGQEHPNARRYRGANRRGLSRGCFGLKLDRIGSMSGLGC-OH;
(SEQ ID NO: 48)
Ac-PGQEHPNARRYRGANRRGLSRGCFGLKLDRIGSMSGLGC-NH₂;
(SEQ ID NO: 46)
Ac-PGQEHPQARRYRGAQRRGLSRGCFGLKLDRIGSMSGLGC-NH₂;
(SEQ ID NO: 49)
Ac-PGQEHPQARKYKGAQKKGLSKGCFGLKLDRIGSMSGLGC-NH₂;
and
(SEQ ID NO: 49)
Ac-PGQEHPQARKYKGAQKKGLSKGCFGLKLDRIGSMSGLGC-OH.

It is further contemplated that the CNP variant includes a conjugate, salt or prodrug of the CNP variants described herein.

In various embodiments, levels of NTproCNP or CXM are measured in a plasma sample, for example, before and after administration of a CNP variant.

In various embodiments, the subject is receiving between 7.5 μg/kg and 30 μg/kg CNP therapy. In various embodiments, the subject is receiving 15 μg/kg or 30 μg/kg CNP therapy. In various embodiments, the dose may be increased to 30 μg/kg or 60 μg/kg.

In various embodiments, the NTproCNP and/or CXM is measured at least 4 hours after administration. In various embodiments, the level of NTproCNP and/or CXM is measured at least 3 months or 6 months after start of CNP therapy. In various embodiments, the level of NTproCNP and/or CXM is measured at least every 3 months, 6 months, or 1 year after start of CNP therapy. In various embodiments, the level of NTproCNP and/or CXM is measured for a duration of at least 3 months, 6 months, 1 year, 2 years, 3 years, 4 years, 5 years, or until puberty/close of growth plates after start of CNP therapy.

In various embodiments, the level of NTproCNP in a sample is compared to a baseline measurement taken prior to start of CNP therapy. In various embodiments, the level of NTproCNP in a sample is compared to average levels in normal control patients.

In various embodiments, CNP therapy dose or frequency is increased when a decrease in NTproCNP indicates an increase in AGV in the subject.

In various embodiments, the level of CXM in a sample is compared to a baseline measurement taken prior to start of CNP therapy. In various embodiments, the level of CXM in a sample is compared to average levels in normal control patients.

In various embodiments, the CXM increase indicates increased bone growth, and wherein the dose of CNP frequency or level is increased when there is CXM increase that enhances AGV.

In various embodiments, the subject is a pediatric subject with open growth plates and received a dose of 15 or 30 μg/kg daily. In various embodiments, the subject is in early adolescence and received a dose increase to 30 μg/kg daily or 60 μg/kg daily. In various embodiments, the subject is an infant and received a dose increase to 30 μg/kg daily.

The disclosure also provides a method of selecting initiation of CNP therapy in a subject comprising i) measuring NTproCNP in the subject at multiple timepoints to establish a baseline NTproCNP level; ii) determining if the NTproCNP levels indicate an SDS within +2 of mean NTproCNP levels; and iii) starting treatment with CNP therapy when the subject has NTproCNP levels below mean NTproCNP SDS. In various embodiments, the subject has an NTproCNP SDS of about −2.5, −2.0, −1.5, −1.0 or −0.5. In various embodiments, CNP therapy is adjusted such that the NTproCNP SDS of the subject is about −0.4, −0.3, −0.2, −0.1, 0, 0.1, 0.2, 0.3, 0.4, 0.5, 0.6, 0.7, 0.8, 0.9, 1.0, 1.1, 1.2, 1.3, 1.4, 0.5, 0.16, 1.7, 1.8, 1.9 or 2.0 after a modulation in CNP therapy dose level and/or frequency.

In certain embodiments, the disclosure contemplates a method of selecting initiation of CNP therapy in a subject having achondroplasia comprising i) measuring NTproCNP in the subject at multiple timepoints to establish a baseline NTproCNP level; ii) determining if the NTproCNP levels indicate an SDS of zero, below zero or above zero; and iii) starting treatment with CNP therapy when the subject has NTproCNP levels above SDS zero.

In various embodiments, NTproCNP is measured at 2 weeks, one month, 3 months, and 6 months prior to CNP therapy to establish a baseline NTproCNP level. In various embodiments, NTproCNP is measured by radioimmunoassay.

Also provided is a method of treating a subject having a bone-related disorder, skeletal dysplasia or short stature described herein comprising,
i) identifying whether a subject has a Loss of Function (LoF) or Gain of Function (GoF) variant of a gene related to short stature;
ii) calculating a polygenic risk score (PRS) of the subject;
iii) determining if the subject has a LoF variant and a PRS in the bottom 20%; and
iv) treating the subject with a CNP variant if the subject has a LoF variant and a PRS in the bottom 20%.

In various embodiments, the subject has a PRS in the bottom 20%, 19%, 18%, 17.5%, 17%, 16.5%, 16%, 15.5%, 15%, 14.5%, 14%, 13.5%, 13%, 12.5%, 12%, 11%, 10%, 9%, 8%, 7.5%, 7%, 6%, 5%, 4%, 3%, 2.5%, 2% or 1%. In various embodiments, step iii) and iv) is a subject with a CNP variant if the subject has a LoF variant and a PRS in the bottom 12.5%.

In various embodiments, the gene related to skeletal dysplasia or short stature is selected from the group consisting of NPR2, SHOX, PTPN11, COL2A1, COL11A1, COL9A2, COL10), aggrecan (ACAN), indian hedgehog (IHH), NPPC, FGFR3, IGF1R, DTL, and pregnancy-associated plasma protein A2 (PAPPA2), or combinations thereof.

Also contemplated is a method of treating a subject having a bone-related disorder, skeletal dysplasia or short stature described herein comprising
i) identifying whether a subject has a NPR2 Loss of Function (LoF) or Gain of Function (GoF) variant;
ii) calculating a polygenic risk score (PRS) of the subject;
iii) determining if the subject has a NPR2 LoF variant and a PRS in the bottom 20%; and
iv) treating the subject with a CNP variant if the subject has a NPR2 LoF variant and a PRS in the bottom 20%.

In various embodiments, the subject has a PRS in the bottom 20%, 19%, 18%, 17.5%, 17%, 16.5%, 16%, 15.5%, 15%, 14.5%, 14%, 13.5%, 13%, 12.5%, 12%, 11%, 10%, 9%, 8%, 7.5%, 7%, 6%, 5%, 4%, 3%, 2.5%, 2% or 1%. In various embodiments, step iii) and iv) is a subject with a CNP variant if the subject has a LoF variant and a PRS in the bottom 12.5%.

In various embodiments, the LoF or GoF variant is determined by a biological activity assay. In various embodiments, a LoF or GoF variant may be predicted based on biological activity and mapping to the predicted 3D structure of the protein, e.g., using AlphaForm 3D mapping or other protein mapping tools.

In various embodiments, the PRS is calculated by a genome-wide association study (GWAS) of height. A PRS is an aggregate genetic score that consists of many, common variant effects of small effect each that are summed across the genome (Choi et al. Nat Protoc, 2020). To calculate a height PRS, Genome Wide Association Study (GWAS) association statistics are obtained to indicate the per-variant strength of association with height. These effect sizes are then applied to an independent sample, here the clinical population, by weighing the number of each height-associated allele (0, 1 or 2) by the GWAS effect size, and summing this weighted count across the genome for each clinical sample. PRS can be interpreted such that an individual with a low PRS carries a lower-than-average number of height-increasing genetic variants, and an individual with a high PRS carries a higher-than-average number of height-increasing variants.

In another aspect, provided herein is a method for increasing facial volume, facial sinus volume, and foramen magnum area in a subject 6 months old or less having a bone-related disorder, skeletal dysplasia or short stature comprising administering CNP variants, conjugates, salts or prodrugs thereof at a dose of at least 30 μg/kg. Also provided is a method of decreasing the incidence of sudden infant death, sleep disordered breathing, and necessity for neurosurgical decompression of the foramen magnum in a subject 6 months old or less having a bone-related disorder, skeletal dysplasia or short stature comprising administering CNP variants, conjugates, salts or prodrugs thereof at a dose of at least 30 μg/kg.

In various embodiments, the increase in facial volume, facial sinus volume, and foramen magnum area are measured by magnetic resonance imaging (MRI). In various embodiments, the change in facial volume, facial sinus volume, and foramen magnum area are compared to baseline levels, healthy control subjects or untreated control subjects.

In various embodiments, the CNP variant is administered subcutaneously. In various embodiments, the CNP variant is administered daily, weekly, every 2 weeks, monthly, or less. In various embodiments, the CNP variant is administered at a dose of 30 μg/kg for 3 months, 6 months, 1 year or more. In various embodiments, the dose of CNP variant is decreased to 15 μg/kg when the subject is about 2 years old.

It is understood that each feature or embodiment, or combination, described herein is a non-limiting, illustrative example of any of the aspects of the invention and, as such, is meant to be combinable with any other feature or embodiment, or combination, described herein. For example, where features are described with language such as “one embodiment”, “some embodiments”, “certain embodiments”, “further embodiment”, “specific exemplary embodiments”, and/or “another embodiment”, each of these types of embodiments is a non-limiting example of a feature that is intended to be combined with any other feature, or combination of features, described herein without having to list every possible combination.

Such features or combinations of features apply to any of the aspects of the invention. Where examples of values falling within ranges are disclosed, any of these examples are contemplated as possible endpoints of a range, any and all numeric values between such endpoints are contemplated, and any and all combinations of upper and lower endpoints are envisioned.

### BRIEF DESCRIPTION OF THE DRAWINGS

FIG. 1A-1C. Annualized growth velocity (AGV) (FIG. 1A), plasma NTproCNP concentrations (FIG. 1B) and NTproCNP SDS (adjusted for age and sex) (FIG. 1C) by cohort across the study. Values are mean±SE. Cohort 1 (6 subjects, age range 6-10yr at screening) received 2.5 μg/kg/d for up to 10 months (˜to day 300), followed by 7.5 μg/kg/d for approximately 2 months (˜to day 360), and thereafter 15 μg/kg/d until study completion. Cohort 2 (6 subjects, age range 5-10) received 7.5 μg/kg/d for the initial 6-8 months (180-240 days)—escalating to 15 μg/kg/d thereafter. Cohorts 3 (8 subjects, age range 6-11) and Cohort 4 (8 subjects, age range 5-8) received 15 μg/kg/d and 30 μg/kg/d respectively throughout the study.

FIG. 2A-2D. Changes in NTproCNP concentration over time by cohort. Individuals within each cohort are delineated. The letter P denotes the time of the visit when the individual was determined to have reached Tanner stage 2. Cohort 1 (FIG. 2A) (6 subjects, age range 6-10yr at screening) received 2.5 μg/kg/d for up to 10 months (˜ to day 300), followed by 7.5 μg/kg/d for approximately 2 months (˜ to day 360), and thereafter 15 μg/kg/d until study completion. Cohort 2 (FIG. 2B) (6 subjects, age range 5-10) received 7.5 μg/kg/d for the initial 6-8 months (180-240 days)—escalating to 15 μg/kg/d thereafter. Cohorts 3 (FIG. 2C) (8 subjects, age range 6-11) and Cohort 4 (FIG. 2D) (8 subjects, age range 5-8) received 15 μg/kg/d and 30 μg/kg/d respectively throughout the study.

FIG. 3. Fold change from baseline (screening) in bone turnover markers (bALP, PINP) and plasma NTproCNP in three Cohort 4 subjects in years 3-4 of therapy. Each panel depicts concurrent analyte concentrations in a single subject.

FIG. 4. Relationship between change (delta) in plasma NTproCNP concentration at 4 hr after injection on Day 183 and NTproCNP SDS prior to injection on the same day.

FIG. 5A-5C. Phase 2 Study 111-202 Growth Velocity and Biomarker Results over Time. Annualized growth velocity (AGV) (FIG. 5A), serum collagen X biomarker (CXM) (FIG. 5B), and serum bone-specific alkaline phosphatase (BSAP) (FIG. 5C), were measured in subjects receiving 2.5, 7.5, 15, or 30 ug/kg/day vosoritide (cohorts 1, 2, 3, and 4). After 6 months of treatment, cohorts 1 and 2 were dose-escalated to 15 ug/kg/day. AGV or biomarker change from baseline are shown on the y-axis, days on vosoritide treatment is shown on the x axis. Lines represent the mean of each cohort, error bars represent the standard error of the mean

FIG. 6A-6C. Natural History Study 111-901 and Phase 3 Study 111-301 Growth Velocity and Biomarker Results over Time. Annualized growth velocity (AGV) (FIG. 6A), serum collagen X biomarker (CXM) (FIG. 6B), and serum bone-specific alkaline phosphatase (BSAP) (FIG. 6C), were measured in untreated subjects in study 111-901, and in the same subjects receiving either 15 ug/kg/day vosoritide or placebo in study 1110301. AGV or serum biomarker concentrations are shown on the y-axis, months from initiation of study 111-301 (vosoritide or placebo treatment) is shown on the x axis. Lines represent the mean of each group, error bars represent the standard error of the mean.

FIG. 7 depicts examples of CNP variant proteins comprising a conjugate moiety.

FIG. 8A illustrates a catchpoint assay which is a competition-based ELISA assay used to measure cGMP (molecular devices). FIG. 8B shows normalized cGMP values for a variety of NPR2 LoF and GoF variants.

FIG. 9A-9B: FIG. 9A) Breakdown of variant activity level based on the predicted consequence for the protein. FIG. 9B) Breakdown of predicted consequences for missense variants based on Combined Annotation Dependent Depletion (CADD) score. FIG. 9C) Comparison of the measured functional activities for NPR2 variants and the average impact on the height of individuals who carry them. X-axis shows the log base 2 activity level with respect to wild type NPR2 (0=wt, −1=½ wt, −2=¼ wt, etc.). Y-axis show the estimated effect size of the variant on adult height. 1 unit of beta corresponds to 1 standard deviation (˜2.5 inches for male, ˜2.2 inches for females).

FIG. 10A-10B. Predicting idiopathic short stature (ISS) based on genetics. FIG. 10A shows the probability of ISS based on polygenic scores for height alone, The first panel shows how the predictive power of polygenic scores changes in the context of NPR2 loss of function variants. FIG. 10B shows predicting ISS by polygenic risk score combined with the presence of an NPR2 Loss of Function variant.

FIGS. 11A-11D. FIG. 11A. 3-D model of NPR2 protein, AF model, low confidence regions shown (yellow and red). FIG. 11B. Depiction of the various domains of NPR2 (dimer) as described in Hannema et al (J Clin Endocrinol Metab. 98: E1988-98, 2013). FIG. 11C. List of variants located in the ligand binding domain were mapped onto the 3D model. FIG. 11D. Summary of all variants (LoF=red, GoF=green) and their potential impact on NPR2 function.

FIGS. 12A-12C. Magnetic Resonance Imaging at Baseline and at Week 52 showing Changes in: (FIG. 12A) Volume of Face; (FIG. 12B) Volume of Sinus; and (FIG. 12C) Area of Foramen Magnum. Changes in Volume of Face (upper panel), Volume of Sinus (middle panel), and in Area of Foramen Magnum (lower panel) on Magnetic Resonance Imaging. The figure shows change from baseline to week 52 in MRI results evaluating the effect of vosoritide on skull morphology. Box plot displays the 25th and 75th percentiles (box edges), the median (midline), mean (open square symbol) and the 2.5th and 97.5th percentiles (whiskers). Dots represent outliers.

### DETAILED DESCRIPTION

The present application pertains to the discovery that biomarkers of bone growth are useful in assessing improvement in bone growth and annualized growth velocity of CNP therapy in subjects having skeletal dysplasia. Biomarkers can be used to determine dose efficacy and modify dose timing and/or frequency of CNP based on the level of biomarkers, such as NTproCNP and CXM. Additional measures of efficacy are provided, including analysis of skull and brain morphology in younger children over time.

### Definitions

As used in the specification and the appended claims, the indefinite articles “a” and “an” and the definite article “the” include plural as well as singular referents unless the context clearly dictates otherwise.

The term “about” or “approximately” means an acceptable error for a particular value as determined by one of ordinary skill in the art, which depends in part on how the value is measured or determined. In certain embodiments, the term “about” or “approximately” means within 1, 2, 3, or 4 standard deviations. In certain embodiments, the term “about” or “approximately” means within 30%, 25%, 20%, 15%, 10%, 9%, 8%, 7%, 6%, 5%, 4%, 3%, 2%, 1%, 0.5%, or 0.05% of a given value or range. Whenever the term “about” or “approximately” precedes the first numerical value in a series of two or more numerical values, it is understood that the term “about” or “approximately” applies to each one of the numerical values in that series.

The term “C-type natriuretic peptide” or “CNP” refers to a small, single chain peptide having a 17-amino acid loop structure at the C-terminal end (GenBank Accession No. NP_077720, for the CNP precursor protein, NPPC) and variants thereof. The 17-mer CNP loop structure, is also referred to as CNP 17, the CNP ring, or CNP cyclic domain. CNP includes the active 53-amino acid peptide (CNP-53) and the mature 22-amino acid peptide (CNP-22), and peptides of varying lengths between the two peptides.

In various embodiments, a “CNP variant” is at least about 40%, 50%, 60%, 70%, 75%, 80%, 85%, 90%, or 95% homologous to the wild type NPPC over the same number of amino acid residues. It is further contemplated that a CNP variant peptide may comprise from about 1 to about 53, or 1 to 39, or 1 to 38, or 1 to 37, or 1 to 35, or 1 to 34, or 1 to 31, or 1 to 27, or 1 to 22, or 10 to 35, or about 15 to about 37 residues of the NPPC polypeptide. In one embodiment, a CNP variant may comprise a sequence of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, or 53 amino acids derived from the NPPC polypeptide. CNP variant also includes conjugates, salts or prodrugs of CNP variants described herein. “CNP therapy” refers to administration of a CNP variant to treat a subject having a bone-related disorder, skeletal dysplasia or short stature as described herein.

The term “conjugate moiety” refers to a moiety that is conjugated to the variant peptide. Conjugate moieties include a lipid, fatty acid, hydrophilic spacer, synthetic polymer, linker, or optionally, combinations thereof.

The term “effective amount” refers to a dosage sufficient to produce a desired result on a health condition, pathology, or disease of a subject or for a diagnostic purpose. The desired result may comprise a subjective or objective improvement in the recipient of the dosage. “Therapeutically effective amount” refers to that amount of an agent effective to produce the intended beneficial effect on health. An appropriate “effective” amount in any individual case may be determined by one of ordinary skill in the art using routine experi-mentation. It will be understood that the specific dose level and frequency of dosage for any particular patient may be varied and will depend upon a variety of factors, including the activity of the specific compound employed; the bioavailability, metabolic stability, rate of excretion and length of action of that compound; the mode and time of administration of the compound; the age, body weight, general health, sex, and diet of the patient; and the severity of the particular condition.

“Substantially pure” or “isolated” means an object species is the predominant species present (i.e., on a molar basis, more abundant than any other individual macromolecular species in the composition), and a substantially purified fraction is a composition wherein the object species comprises at least about 50% (on a molar basis) of all macromolecular species present. In one embodiment, a substantially pure composition means that the species of interest comprises at least about 70%, 75%, 80%, 85%, 90%, 95%, 98% or more of the macromolecular species present in the composition on a molar or weight basis. The object species is purified to essential homogeneity (contaminant species cannot be detected in the composition by conventional detection methods) if the composition consists essentially of a single macromolecular species. Solvent species, small molecules (<500 Daltons), stabilizers (e.g., BSA), and elemental ion species are not considered macromolecular species for purposes of this definition. In an embodiment, the compounds of the disclosure are substantially pure or isolated. In another embodiment, the compounds of the disclosure are substantially pure or isolated with respect to the macromolecular starting materials used in their production. In yet another embodiment, the pharmaceutical compositions of the disclosure comprise a substantially pure or isolated CNP variant admixed with one or more pharmaceutically acceptable excipients, carriers or diluents, and optionally with another biologically active agent.

“Treatment” refers to prophylactic treatment or therapeutic treatment or diagnostic treatment. In certain embodiments, “treatment” refers to administration of a compound or composition to a subject for therapeutic, prophylactic or diagnostic purposes.

A “prophylactic” treatment is a treatment administered to a subject who does not exhibit signs of a disease or exhibits only early signs of the disease, for the purpose of decreasing the risk of developing pathology. The compounds or compositions of the disclosure may be given as a prophylactic treatment to reduce the likelihood of developing a pathology or to minimize the severity of the pathology, if developed.

A “therapeutic” treatment is a treatment administered to a subject who exhibits signs or symptoms of pathology for the purpose of diminishing or eliminating those signs or symptoms. The signs or symptoms may be biochemical, cellular, histological, functional or physical, subjective or objective. The compounds of the disclosure may also be given as a therapeutic treatment or for diagnosis.

“Diagnostic” means identifying the presence, extent and/or nature of a pathologic condition. Diagnostic methods differ in their specificity and selectivity. While a particular diagnostic method may not provide a definitive diagnosis of a condition, it suffices if the method provides a positive indication that aids in diagnosis.

“Bone- or cartilage-associated biomarker” or “bone- or cartilage-associated marker” refers to a growth factor, enzyme, protein, or other detectable biological substance or moiety whose level is increased or decreased in association with, e.g., cartilage turnover, cartilage formation, cartilage growth, bone resorption, bone formation, bone growth, or combinations thereof. Such biomarkers may be measured before, during and/or after administration of a CNP variant as described herein. Exemplary bone- or cartilage-associated biomarkers include, but are not limited to, CNP, cGMP, propeptides of collagen type II and fragments thereof, collagen type II and fragments thereof, propeptides of collagen type I and fragments thereof, collagen type I and fragments thereof, osteocalcin, proliferating cell nuclear antigen (PCNA), aggrecan chondroitin sulfate, collagen X, N terminal fragment of collagen X (CXM) and alkaline phosphatase. Cartilage- and bone-associated biomarkers can be measured in any appropriate biological sample, including but not limited to tissues, blood, serum, plasma, cerebrospinal fluid, synovial fluid and urine.

“Pharmaceutical composition” or “formulation” refers to a composition suitable for pharmaceutical use in subject animal, including humans and mammals. A pharmaceutical composition comprises a therapeutically effective amount of CNP variant, optionally another biologically active agent, and optionally a pharmaceutically acceptable excipient, carrier or diluent. In an embodiment, a pharmaceutical composition encompasses a composition comprising the active ingredient(s), and the inert ingredient(s) that make up the carrier, as well as any product that results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present disclosure encompass any composition made by admixing a compound of the disclosure and a pharmaceutically acceptable excipient, carrier or diluent.

“Pharmaceutically acceptable carrier” refers to any of the standard pharmaceutical carriers, buffers, and the like, such as a phosphate buffered saline solution, 5% aqueous solution of dextrose, and emulsions (e.g., an oil/water or water/oil emulsion). Non-limiting examples of excipients include adjuvants, binders, fillers, diluents, disintegrants, emulsifying agents, wetting agents, lubricants, glidants, sweetening agents, flavoring agents, and coloring agents. Suitable pharmaceutical carriers, excipients and diluents are described in Remington's Pharmaceutical Sciences, 19th Ed. (Mack Publishing Co., Easton, 1995). Preferred pharmaceutical carriers depend upon the intended mode of administration of the active agent. Typical modes of administration include enteral (e.g., oral) or parenteral (e.g., subcutaneous, intramuscular, intravenous or intraperitoneal injection; or topical, transdermal, or transmucosal administration).

A “pharmaceutically acceptable salt” is a salt that can be formulated into a compound for pharmaceutical use, including but not limited to metal salts (e.g., sodium, potassium, magnesium, calcium, etc.) and salts of ammonia or organic amines.

By “pharmaceutically acceptable” or “pharmacologically acceptable” is meant a material that is not biologically or otherwise undesirable, i.e., the material may be administered to an individual without causing any undesirable biological effects or without interacting in a deleterious manner with any of the components of the composition in which it is contained or with any components present on or in the body of the individual.

“Physiological conditions” refer to conditions in the body of an animal (e.g., a human). Physiological conditions include, but are not limited to, body temperature and an aqueous environment of physiologic ionic strength, pH and enzymes. Physiological conditions also encompass conditions in the body of a particular subject which differ from the “normal” conditions present in the majority of subjects, e.g., which differ from the normal human body temperature of approximately 37° C. or differ from the normal human blood pH of approximately 7.4.

By “physiological pH” or a “pH in a physiological range” is meant a pH in the range of approximately 7.0 to 8.0 inclusive, more typically in the range of approximately 7.2 to 7.6 inclusive.

As used herein, the term “subject” encompasses mammals and non-mammals. Examples of mammals include, but are not limited to, any member of the mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish, and the like. The term does not denote a particular age or gender. In various embodiments, the subject is human. In various embodiments the subject is a child or adolescent. In various embodiments, the subject is an infant. In various embodiments, the subject is older than 3, older than 2, older than 1, or older than 6 months in age.

### C-Type Natriuretic Peptide

C-type natriuretic peptide (CNP) (Biochem. Biophys. Res. Commun., 168:863-870 (1990) (GenBank Accession No. NP_077720, for the CNP precursor protein, NPPC) (J. Hypertens., 10:907-912 (1992)) is a small, single chain peptide in a family of peptides (ANP, BNP, CNP) having a 17-amino acid loop structure (Levin et al., N. Engl. J. Med., 339:863-870 (1998)) and have important roles in multiple biological processes. CNP interacts with natriuretic peptide receptor-B (NPR-B, GC-B) to stimulate the generation of cyclic-guanosine monophosphate (cGMP) (J. Hypertens., 10:1111-1114 (1992)). CNP is expressed more widely, including in the central nervous system, reproductive tract, bone and endothelium of blood vessels (Gardner et al., Hypertension, 49:419-426 (2007)).

In humans, CNP is initially produced from the natriuretic peptide precursor C (NPPC) gene as a single chain 126-amino acid pre-pro polypeptide (Sudoh et al., Biochem. Biophys. Res. Commun., 168:863-870 (1990)). Removal of the signal peptide yields pro-CNP, and further cleavage by the endoprotease furin generates an active 53-amino acid peptide (CNP-53), which is secreted and cleaved again by an unknown enzyme to produce the mature 22-amino acid peptide (CNP-22) (Wu, J. Biol. Chem. 278:25847-852 (2003)). CNP-53 and CNP-22 differ in their distribution, with CNP-53 predominating in tissues, while CNP-22 is mainly found in plasma and cerebrospinal fluid (J. Alfonzo, Recept. Signal. Transduct. Res., 26:269-297 (2006)). Both CNP-53 and CNP-22 bind similarly to NPR-B.

Downstream signaling mediated by cGMP generation influences a diverse array of biological processes that include endochondral ossification. For example, knockout of either CNP or NPR-B in mouse models results in animals having a dwarfed phenotype with shorter long bones and vertebrae. Mutations in human NPR-B that block proper CNP signaling have been identified and result in dwarfism (Olney, et al., J. Clin. Endocrinol. Metab. 91 (4): 1229-1232 (2006); Bartels, et al., Am. J. Hum. Genet. 75:27-34 (2004)). In contrast, mice engineered to produce elevated levels of CNP display elongated long bones and vertebrae.

Natural CNP gene and polypeptide have been previously described. U.S. Pat. No. 5,352,770 discloses isolated and purified CNP-22 from porcine brain identical in sequence to human CNP and its use in treating cardiovascular indications. U.S. Pat. No. 6,034,231 discloses the human gene and polypeptide of pre-proCNP (126 amino acids) and the human CNP-53 gene and polypeptide. The mature CNP is a 22-amino acid peptide (CNP-22). Certain CNP variants are disclosed in U.S. Pat. No. 8,198,242, incorporated by reference herein.

In various embodiments, CNP of the disclosure includes truncated CNP ranging from human CNP-17 (hCNP-17) to human CNP-53 (hCNP-53), and having wild-type amino acid sequences derived from hCNP-53 and also variants thereof. Such truncated CNP peptides include:

(Pro-Gly-CNP37)
(SEQ ID NO: 1)
PGQEHPNARKYKGANKKGLSKGCFGLKLDRIGSMSGLGC;
(Gly-CNP-37)
(SEQ ID NO: 2)
GQEHPNARKYKGANKKGLSKGCFGLKLDRIGSMSGLGC;
(Gly-CNP53)
(SEQ ID NO: 3)
GDLRVDTKSRAAWARLLQEHPNARKYKGANKKGLSKGCFGLKLDRIGSM
SGLGC;
(Pro-CNP53)
(SEQ ID NO: 4)
PDLRVDTKSRAAWARLLQEHPNARKYKGANKKGLSKGCFGLKLDRIGSM
SGLGC;
(Met-CNP53)
(SEQ ID NO: 5)
MDLRVDTKSRAAWARLLQEHPNARKYKGANKKGLSKGCFGLKLDRIGSM
SGLGC;
[CNP-53(M48N)]
(SEQ ID NO: 6)
DLRVDTKSRAAWARLLQEHPNARKYKGANKKGLSKGCFGLKLDRIGSNS
GLGC;
(CNP-52)
(SEQ ID NO: 7)
LRVDTKSRAAWARLLQEHPNARKYKGANKKGLSKGCFGLKLDRIGSMSG
LGC;
(CNP-51)
(SEQ ID NO: 8)
RVDTKSRAAWARLLQEHPNARKYKGANKKGLSKGCFGLKLDRIGSMSGL
GC;
(CNP-50)
(SEQ ID NO: 9)
VDTKSRAAWARLLQEHPNARKYKGANKKGLSKGCFGLKLDRIGSMSGLG
C;
(CNP-49)
(SEQ ID NO: 10)
DTKSRAAWARLLQEHPNARKYKGANKKGLSKGCFGLKLDRIGSMSGLG
C;
(CNP-48)
(SEQ ID NO: 11)
TKSRAAWARLLQEHPNARKYKGANKKGLSKGCFGLKLDRIGSMSGLGC;
(CNP-47)
(SEQ ID NO: 12)
KSRAAWARLLQEHPNARKYKGANKKGLSKGCFGLKLDRIGSMSGLGC;
(CNP-46)
(SEQ ID NO: 13)
SRAAWARLLQEHPNARKYKGANKKGLSKGCFGLKLDRIGSMSGLGC;
(CNP-45)
(SEQ ID NO: 14)
RAAWARLLQEHPNARKYKGANKKGLSKGCFGLKLDRIGSMSGLGC;
(CNP-44)
(SEQ ID NO: 15)
AAWARLLQEHPNARKYKGANKKGLSKGCFGLKLDRIGSMSGLGC;
(CNP-43)
(SEQ ID NO: 16)
AWARLLQEHPNARKYKGANKKGLSKGCFGLKLDRIGSMSGLGC;
(CNP-42)
(SEQ ID NO: 17)
WARLLQEHPNARKYKGANKKGLSKGCFGLKLDRIGSMSGLGC;
(CNP-41)
(SEQ ID NO: 18)
ARLLQEHPNARKYKGANKKGLSKGCFGLKLDRIGSMSGLGC;
(CNP-40)
(SEQ ID NO: 19)
RLLQEHPNARKYKGANKKGLSKGCFGLKLDRIGSMSGLGC;
(CNP-39)
(SEQ ID NO: 20)
LLQEHPNARKYKGANKKGLSKGCFGLKLDRIGSMSGLGC;
(CNP-38)
(SEQ ID NO: 21)
LQEHPNARKYKGANKKGLSKGCFGLKLDRIGSMSGLGC;
(CNP-37)
(SEQ ID NO: 22)
QEHPNARKYKGANKKGLSKGCFGLKLDRIGSMSGLGC;
(CNP-36)
(SEQ ID NO: 23)
EHPNARKYKGANKKGLSKGCFGLKLDRIGSMSGLGC;
(CNP-35)
(SEQ ID NO: 24)
HPNARKYKGANKKGLSKGCFGLKLDRIGSMSGLGC;
(CNP-34)
(SEQ ID NO: 25)
PNARKYKGANKKGLSKGCFGLKLDRIGSMSGLGC;
(CNP-33)
(SEQ ID NO: 26)
NARKYKGANKKGLSKGCFGLKLDRIGSMSGLGC;
(CNP-32)
(SEQ ID NO: 27)
ARKYKGANKKGLSKGCFGLKLDRIGSMSGLGC;
(CNP-31)
(SEQ ID NO: 28)
RKYKGANKKGLSKGCFGLKLDRIGSMSGLGC;
(CNP-30)
(SEQ ID NO: 29)
KYKGANKKGLSKGCFGLKLDRIGSMSGLGC;
(CNP-29)
(SEQ ID NO: 30)
YKGANKKGLSKGCFGLKLDRIGSMSGLGC;
(CNP-28)
(SEQ ID NO: 31)
KGANKKGLSKGCFGLKLDRIGSMSGLGC;
(CNP-27)
(SEQ ID NO: 32)
GANKKGLSKGCFGLKLDRIGSMSGLGC;
(CNP-26)
(SEQ ID NO: 33)
ANKKGLSKGCFGLKLDRIGSMSGLGC;
(CNP-25)
(SEQ ID NO: 34)
NKKGLSKGCFGLKLDRIGSMSGLGC;
(CNP-24)
(SEQ ID NO: 35)
KKGLSKGCFGLKLDRIGSMSGLGC;
(CNP-23)
(SEQ ID NO: 36)
KGLSKGCFGLKLDRIGSMSGLGC;
(CNP-21)
(SEQ ID NO: 37)
LSKGCFGLKLDRIGSMSGLGC;
(CNP-20)
(SEQ ID NO: 38)
SKGCFGLKLDRIGSMSGLGC;
(CNP-19)
(SEQ ID NO: 39)
KGCFGLKLDRIGSMSGLGC;
(CNP-18)
(SEQ ID NO: 40)
GCFGLKLDRIGSMSGLGC;
[CNP-37(M32N)]
(SEQ ID NO: 41)
QEHPNARKYKGANKKGLSKGCFGLKLDRIGSNSGLGC;
(Pro-CNP-37)
(SEQ ID NO: 42)
PQEHPNARKYKGANKKGLSKGCFGLKLDRIGSMSGLGC;
(Met-CNP-37)
(SEQ ID NO: 43)
MQEHPNARKYKGANKKGLSKGCFGLKLDRIGSMSGLGC;
[Gly-CNP-37(M32N)]
(SEQ ID NO: 44)
GQEHPNARKYKGANKKGLSKGCFGLKLDRIGSNSGLGC;
(Met-Gly-CNP-37)
(SEQ ID NO: 45)
MGQEHPNARKYKGANKKGLSKGCFGLKLDRIGSMSGLGC;
(SEQ ID NO: 46)
PGQEHPQARRYRGAQRRGLSRGCFGLKLDRIGSMSGLGC;
(SEQ ID NO: 47)
PGQEHPNARKYKGANKKGLSKGCFGLKLDRIGSMSGLGC;
(SEQ ID NO: 48)
PGQEHPNARRYRGANRRGLSRGCFGLKLDRIGSMSGLGC;
and
(SEQ ID NO: 49)
PGQEHPQARKYKGAQKKGLSKGCFGLKLDRIGSMSGLGC.

In various embodiments, the CNP variant peptides are modified CNP-37 or CNP-38 peptides, optionally having mutation(s)/substitution(s) at the furin cleavage site, and/or containing glycine or proline-glycine at the N-terminus. Exemplary CNP-37 variants include but are not limited to:

[CNP-37(M32N); SEQ ID NO: 41]
QEHPNARKYKGANKKGLSKGCFGLKLDRIGSNSGLGC;
(Met-CNP-37; SEQ ID NO: 43)
MQEHPNARKYKGANKKGLSKGCFGLKLDRIGSMSGLGC;
(Pro-CNP-37; SEQ ID NO: 42)
PQEHPNARKYKGANKKGLSKGCFGLKLDRIGSMSGLGC;
[Gly-CNP-37 (M32N); SEQ ID NO: 44]
GQEHPNARKYKGANKKGLSKGCFGLKLDRIGSNSGLGC;
(Pro-Gly-CNP-37; SEQ ID NO: 1)
PGQEHPNARKYKGANKKGLSKGCFGLKLDRIGSMSGLGC;
(Met-Gly-CNP-37; SEQ ID NO: 45)
MGQEHPNARKYKGANKKGLSKGCFGLKLDRIGSMSGLGC;
(Gly-CNP-37: SEQ ID NO: 2)
GQEHPNARKYKGANKKGLSKGCFGLKLDRIGSMSGLGC
(SEQ ID NO: 50)
GQEHPNARKYKGANPKGLSKGCFGLKLDRIGSMSGLGC;
(SEQ ID NO: 51)
GQEHPNARKYKGANQKGLSKGCFGLKLDRIGSMSGLGC;
(SEQ ID NO: 52)
GQEHPNARKYKGANQQGLSKGCFGLKLDRIGSMSGLGC;
and
(SEQ ID NO: 53)
GQEHPNARKYKGANKPGLSKGCFGLKLDRIGSMSGLGC;
In various embodiments, CNP variants of the disclosure include
(SEQ ID NO: 46)
PGQEHPQARRYRGAQRRGLSRGCFGLKLDRIGSMSGLGC;
(SEQ ID NO: 47)
PGQEHPNARKYKGANKKGLSKGCFGLKLDRIGSMSGLGC;
(SEQ ID NO: 48)
PGQEHPNARRYRGANRRGLSRGCFGLKLDRIGSMSGLGC;
or
(SEQ ID NO: 49)
PGQEHPQARKYKGAQKKGLSKGCFGLKLDRIGSMSGLGC.

The variant peptide may further comprise an acetyl group. In various embodiments, the acetyl group is on the N-terminus of the peptide. In various embodiments, the peptide further comprises an OH or an NH₂ group at the C-terminus.

The variant peptide may comprise a conjugate moiety. In various embodiments, the conjugate moiety is on a residue of the CNP cyclic domain or at a site other than the CNP cyclic domain. In various embodiments, the conjugate moiety is on a lysine residue. In various embodiments, the conjugate moiety comprises one or more acid moieties. In various embodiments, the acid moiety is a hydrophobic acid.

In various embodiments, the variant has the structure:

(SEQ ID NO: 46)
PGQEHPQARRYRGAQRRGLSRGCFGLK(AEEA-AEEA-γGlu-C18DA)
LDRIGSMSGLGC,
or
(SEQ ID NO: 46)
Ac-PGQEHPQARRYRGAQRRGLSRGCFGLK(AEEA-AEEA-γGlu-
C18DA)LDRIGSMSGLGC-OH.

In various embodiments, the variant is selected from the group consisting of

(SEQ ID NO: 46)
Ac-PGQEHPQARRYRGAQRRGLSRGCFGLKLDRIGSMSGLGC-OH;
(SEQ ID NO: 47)
Ac-PGQEHPNARKYKGANKKGLSKGCFGLKLDRIGSMSGLGC-NH₂;
(SEQ ID NO: 48)
Ac-PGQEHPNARRYRGANRRGLSRGCFGLKLDRIGSMSGLGC-OH;
(SEQ ID NO: 48)
Ac-PGQEHPNARRYRGANRRGLSRGCFGLKLDRIGSMSGLGC-NH₂;
(SEQ ID NO: 46)
Ac-PGQEHPQARRYRGAQRRGLSRGCFGLKLDRIGSMSGLGC-NH₂;
(SEQ ID NO: 49)
Ac-PGQEHPQARKYKGAQKKGLSKGCFGLKLDRIGSMSGLGC-NH₂;
and
(SEQ ID NO: 49)
Ac-PGQEHPQARKYKGAQKKGLSKGCFGLKLDRIGSMSGLGC-OH.

In various embodiments, the CNP variant is Ac-PGQEHPQARRYRGAQRRGLSRGCFGLK (AEEA-AEEA-γGlu-C18DA) LDRIGSMSGLGC-OH (SEQ ID NO: 46). In various embodiments, the CNP variant is Ac-PGQEHPNARKYKGANKKGLSKGCFGLK (AEEA-AEEA-γGlu-C18DA) LDRIGSMSGLGC-OH (SEQ ID NO: 47). In various embodiments, the CNP variant is PGQEHPNARKYKGANKKGLSKGCFGLK (AEEA-AEEA-γGlu-C18DA) LDRIGSMSGLGC-OH (SEQ ID NO: 47).

It is further contemplated that the CNP variant is conjugated to or is complexed to a moiety, e.g., a conjugate moiety, that confers increased stability or half-life. In various embodiments, the conjugate moiety is complexed via a non-covalent bond or is attached by a covalent bond. The moiety may be non-covalently attached with the peptide via electrostatic interactions. Alternatively, the moiety may be covalently associated to the peptide via one or more linker moieties. Linkers can be cleavable and non-cleavable linkers. Cleavable linkers may be cleaved via enzymes, nucleophilic/basic reagents, reducing agents, photo-irradiation, electrophilic/acidic reagents, organometallic and metal reagents, or oxidizing reagents. Linkers may also be self-immolative linkers. Exemplary linkers include, but are not limited to, N-succinimidyl-3-(2-pyridyldithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis (p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene), beta alanine, 4-aminobutyric acid (GABA), 2-aminoethoxy acid (AEA), aminoethoxy-2-ethoxy acetic acid (AEEA), 5 aminovaleric acid (AVA), 6-aminocaproic acid (Abx), a vicinal diol cleavable linker, Trimethyl Lock Lactonization, p-alkoxyphenyl carbamate, bicin, peptoid or bicin-type linkers, and electronic linkers as described herein.

It is contemplated that the linker is attached to a residue of the CNP variant within the CNP cyclic domain or at a site other than the CNP cyclic domain. In various embodiments, the linker is attached to a lysine residue. In various embodiments, the linker is attached to a lysine residue in the CNP cyclic domain.

In various embodiments, the CNP variant is attached to the conjugate moiety via the linker. In various embodiments, the linker is attached to the conjugate moiety via the hydrophilic spacer of the conjugate moiety.

In various embodiments, the linker is a hydrolysable linker.

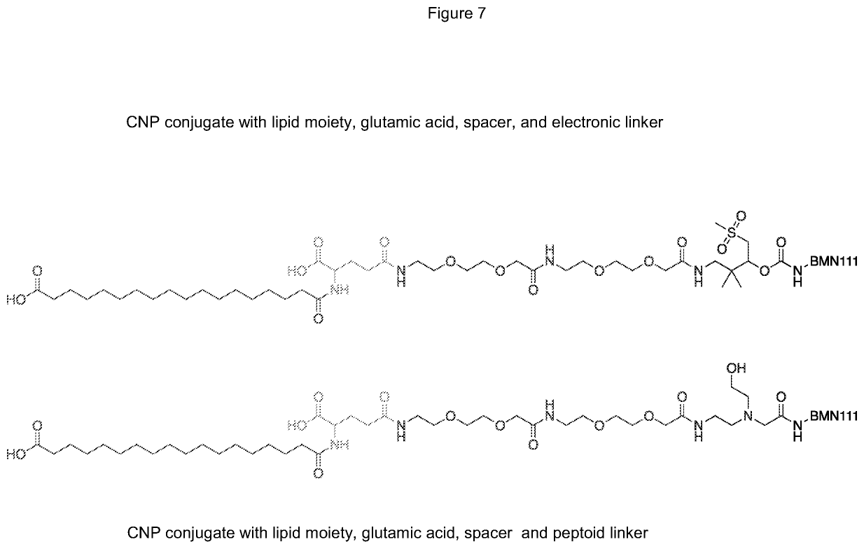

In various embodiments the linker is a peptoid or electronic linker. In various embodiments the linker is a peptoid linker. In various embodiments the linker is an electronic linker. In various embodiments, the linker comprises an SO₂ moiety. Exemplary linkers are illustrated in FIG. 7. It is further contemplated that linkers in FIG. 7 are modified by substitution on the R groups. For example, bicin-type linkers include the structures as set out below:

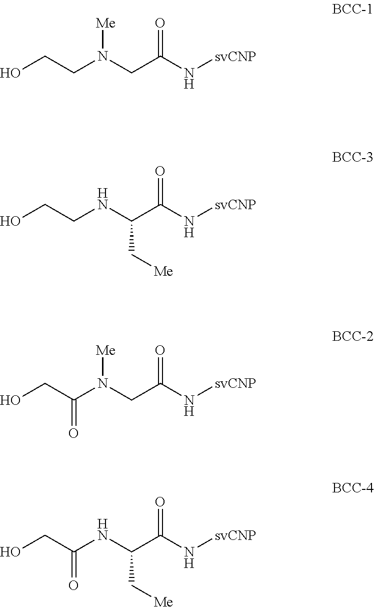

In various embodiments, the moiety conjugated to the peptide is a synthetic polymer such as polyethylene glycol, a linker, a lipid moiety or fatty acid, or a combination thereof. In various embodiments, the CNP variant is conjugated with a fatty acid, an amino acid, a spacer and a linker. In various embodiments, the CNP variant is conjugated with a fatty acid, an amino acid, a polyethylene glycol spacer or a polyethylene glycol derivative spacer, and a linker. In various embodiments, the CNP variant is conjugated with a fatty acid, an amino acid, a spacer, and a linker, wherein the spacer comprises a substituted C-6 to C-20 alkyl chain or any amino acid, or a combination of both, wherein the carbon atoms of the alkyl chain can be replaced by one or more of O, NH, N (C-1 to C-6 alkyl), or carbonyl groups.

In various embodiments, the CNP variant is conjugated with a fatty acid. It is hypothesized that the lipid technology increases the serum half-life of the CNP variant allowing for less frequent injections and/or improved oral delivery. In various embodiments, the fatty acid is a short chain, medium chain, long chain fatty acid, or a dicarboxylic fatty acid. In various embodiments, the fatty acid is saturated or unsaturated. In various embodiments, the fatty acid is a C-6 to C-20 fatty acid. In various embodiments, the fatty acid is a C-6, C-8, C-10, C-12, C-14, C-16, C-18 or C-20 fatty acid. In various embodiments, the fatty acid is decanoic acid, dodecanoic acid, myristic acid, palmitic acid, stearic acid, arachidic acid, or diacids of the same. In various embodiments, the fatty acid is conjugated to a lysine residue.

In various embodiments, it is contemplated that the CNP variants described herein comprise a conjugate moiety as described herein. It is contemplated that the conjugate moiety is on a residue of the CNP cyclic domain or at a site other than the CNP cyclic domain. In various embodiments, the conjugate moiety is on a lysine residue. In various embodiments, the conjugate moiety comprises one or more acid moieties. In various embodiments, the acid moiety is a fatty acid. Exemplary CNP variants and peptide conjugates are described in International Patent Application No. PCT/US2020/051100 and USSN 17/642,150, incorporated by reference herein in their entirety. Variants, conjugates and salts of CNP are disclosed in U.S. Ser. No. 17/634,034, herein incorporated by reference.

In various embodiments, the conjugate moiety comprises an acid moiety linked to a hydrophilic spacer. In various embodiments, the hydrophilic spacer is a substituted C-6 to C-20 alkyl chain or any amino acid, or a combination of both, wherein the carbon atoms of the alkyl chain can be replaced by one or more of O, NH, N (C-1 to C-6 alkyl), or carbonyl groups. In various embodiments, the hydrophilic spacer is any amino acid. In various embodiments, the hydrophilic spacer is gamma glutamic acid (γGlu). In various embodiments, the hydrophilic spacer is a substituted C-6 to C-20 alkyl chain. In various embodiments, the hydrophilic spacer is a substituted C-6, C-8, C-10, C-12, C-14, C-16, C-18 or C-20 alkyl chain. In various embodiments, the hydrophilic spacer is a substituted C-9 to C-18 alkyl chain. In various embodiments, the hydrophilic spacer is a substituted C-18 alkyl chain. In various embodiments, the hydrophilic spacer is a substituted C-9 alkyl chain. In various embodiments, the hydrophilic spacer is one or more OEG (8-amino-3,6-dioxaoctanoic acid) groups. In various embodiments, the hydrophilic spacer is one or two OEG (8-amino-3,6-dioxaoctanoic acid) groups. In various embodiments, the hydrophilic spacer is OEG (8-amino-3,6-dioxaoctanoic acid). In various embodiments, the spacer is OEG (8-amino-3,6-dioxaoctanoic acid) or γGlu. In various embodiments, the hydrophilic spacer is gamma glutamic acid (γGlu) linked to one or more OEG (8-amino-3,6-dioxaoctanoic acid) groups. In various embodiments, the hydrophilic spacer is gamma glutamic acid (γGlu) linked to one or two OEG (8-amino-3,6-dioxaoctanoic acid) groups (diEG). In various embodiments, the acid moiety and the hydrophilic spacer have the structure AEEA-AEEA-γGlu-C18DA.

In various embodiments, the disclosure contemplates use of CNP variants comprising hydrophilic or water soluble polymers (e.g., oxygenated alkyl chains, wherein the carbon atoms can be replaced with one or more oxygen atoms, such as polyethylene glycol (PEG) or polyethylene oxide (PEO) and the like). In various embodiments, the water soluble polymers can vary in type (e.g., homopolymer or copolymer; random, alternating or block copolymer; linear or branched; monodispersed or polydispersed), linkage (e.g., hydrolysable or stable linkage such as, e.g., amide, imine, aminal, alkylene, or ester bond), conjugation site (e.g., at the N-terminus, internal, and/or C-terminus), and length (e.g., from about 0.2, 0.4 or 0.6 kDa to about 2, 5, 10, 25, 50 or 100 kDa). The hydrophilic or water-soluble polymer can be conjugated to the CNP variant by means of N-hydroxy succinimide (NHS)—or aldehyde-based chemistry or other chemistry, as is known in the art. In various embodiments, negatively charged PEG-CNP variants can be designed for reduced renal clearance, including but not limited to use of carboxylated, sulfated and phosphorylated compounds (Caliceti, Adv. Drug Deliv. Rev., 55:1261-77 (2003); Perlman, J. Clin. Endo. Metab., 88:3227-35 (2003); Pitkin, Antimicrob. Ag. Chemo., 29:440-444 (1986); Vehaskari, Kidney Int'l, 22:127-135 (1982)). In one embodiment, the PEG (or PEO) moiety contains carboxyl group(s), sulfate group(s), and/or phosphate group(s).

In another embodiment, the hydrophilic polymer (e.g., PEG or PEO) moieties conjugated to the N-terminus, C-terminus and/or internal site(s) of CNP variants described herein contain one or more functional groups that are positively charged under physiological conditions. Such moieties are designed, inter alia, to improve distribution of such conjugated CNP variants to cartilage tissues. In one embodiment, PEG moieties contain one or more primary, secondary or tertiary amino groups, quaternary ammonium groups, and/or other amine-containing (e.g., urea) groups.

### Methods of Using CNP Variants

Achondroplasia is a result of an autosomal dominant mutation in the gene for fibroblast growth factor receptor 3 (FGFR-3), which causes an abnormality of cartilage formation. FGFR-3 normally has a negative regulatory effect on chondrocyte growth, and hence bone growth. In achondroplasia, the mutated form of FGFR-3 is constitutively active, which leads to severely shortened bones. In humans activating mutations of FGFR-3 are the primary cause of genetic dwarfism. Mice having activated FGFR-3 serve as a model of achondroplasia, the most common form of the skeletal dysplasias, and overexpression of CNP rescues these animals from dwarfism. Accordingly, functional variants of CNP are potential therapeutics for treatment of the various skeletal dysplasias.

By stimulating matrix production, proliferation and differentiation of chondrocytes and increasing long bone growth, the CNP variants of the disclosure are useful for treating mammals, including humans, suffering from a bone-related disorder, such as a skeletal dysplasia or short stature. Non-limiting examples of CNP-responsive bone-related disorders skeletal dysplasias and short stature disorders include achondroplasia, hypochondroplasia, short stature, dwarfism, osteochondrodysplasias, thanatophoric dysplasia, osteogenesis congenita, achondrogenesis, chondrodysplasia congenit, homozygous achondroplasia, chondrodysplasia congenit, camptomelic dysplasia, congenital lethal hypophosphatasia, perinatal lethal type of osteogenesis congenita, short-rib polydactyly syndromes, hypochondroplasia, rhizomelic type of chondrodysplasia congenit, Jansen-type metaphyseal dysplasia, spondyloepiphyseal dysplasia congenital, atelosteogenesis, diastrophic dysplasia, congenital short femur, Langer-type mesomelic dysplasia, Nievergelt-type mesomelic dysplasia, Robinow syndrome, Reinhardt syndrome, acrodysostosis, peripheral dysostosis, Kniest dysplasia, fibrochondrogenesis, Roberts syndrome, acromesomelic dysplasia, micromelia, Morquio syndrome, Kniest syndrome, metatrophic dysplasia, and spondyloepimetaphyseal dysplasia, disorders related to NPR2 mutation, SHOX mutation (Turner's syndrome/Leri Weill), PTPN11 mutations (Noonan's syndrome) and IGF1R mutation.

Additional short stature and growth plate disorders contemplated by the methods include disorders related to mutations in collagen (COL2A1, COL11A1, COL9A2, COL10), aggrecan (ACAN), indian hedgehog (IHH), PTPN11, NPR2, NPPC, FGFR3, or IGF1R.

Further, the CNP variants are useful as an adjunct or alternative to growth hormone for treating idiopathic short stature and other skeletal dysplasias.

Growth plate disorders include disorders that result in short stature or abnormal bone growth and that may be the result of a genetic mutation in a gene involved in bone growth, including collagen (COL2A1, COL11A1, COL9A2, COL10), aggrecan (ACAN), indian hedgehog (IHH), PTPN11, NPR2, NPPC, FGFR3 or IGF1R. In various embodiments, the growth plate disorder or short stature is associated with one or more mutations in a gene associated with a RASopathy. In various embodiments, a subject with a growth plate disorder is heterozygous for a mutation in a growth plate gene. In various embodiments, the mutation is a loss-of-function mutation. In various embodiments, the mutation is a gain-of-function mutation. Growth plate disorders include, but are not limited to, familial short stature, dominant familial short stature which is also known as dominant inherited short stature, or idiopathic short stature. See, e.g., Plachy et al., J Clin Endocrinol Metab 104:4273-4281, 2019.

Mutations in ACAN can give rise to familial osteochondritis dissecans and short stature and eventually osteoarthritis, characterized by areas of bone damage (or lesions) caused by the detachment of cartilage and sometimes bone from the end of the bone at a joint. It has been suggested that the disorganized cartilage network in growing bones impairs their growth, leading to short stature. A mutation associated with ACAN and short stature includes Val2303Met. See Stattin et al., Am J Hum Genet 86 (2): 126-37, 2010. It is contemplated that patients with a mutation in ACAN resulting in short stature would benefit from treatment with CNP as administration may be able to increase height in these patients by the known interaction of CNP with FGFR3.

The natriuretic peptide system, including receptor NPR2, has been shown to be involved in regulation of endochondral bone growth (Vasques et al., Horm Res Pediat 82:222-229, 2014). Studies have shown that homozygous or compound heterozygous loss-of-function mutations in NPR2 cause acromesomelic dysplasia type Maroteaux (AMDM), which is a skeletal dysplasia having extremely short stature (Vasquez et al., 2014, supra). There are reports implicating heterozygous loss-of-function (such as dominant negative) NPR2 mutations as a cause of short stature, whereas gain-of-function NPR2 heterozygous mutations have been found to be responsible for tall stature (Vasquez et al., 2014, supra). In view of CNP's interaction with NPR2 to stimulate cGMP generation, increasing cGMP levels is desirable in these conditions and would have therapeutic benefit in the management of the complications from these diseases and conditions.

Heterozygous mutations of NPR2 are believed to result in idiopathic short stature and other forms of short stature. Mutations in the NPR2 gene are set out below and described in Amano et al., J Clin Endocrinol Metab 99: E713-718, 2014, Hisado-Oliva et al., J Clin Endocrinol Metab 100: E1133-1142, 2015 and Vasques et al., J Clin Endocrinol Metab 98: E1636-1644, 2013, hereby incorporated by reference. It is contemplated that a subject having short stature to be treated with a CNP variant as described herein has a height SDS of less than −1.0, −1.5, −2.0, −2.5, or −3.0, and has at least one parent with a height SDS of less than −1.0, −1.5, −2.0 or −2.5, optionally wherein the second parent has height within the normal range. In various embodiments, the CNP variants are useful to treat a subject with short stature having a height SDS of between −2.0 to −3.0. In various embodiments, the CNP variants are useful to treat a subject with short stature having a height SDS of between −2.0 to −2.5. However, because de novo mutations in NPR2 can result in short stature as defined by a height SDS of less than −1.5, −2.0, −2.5, or −3.0, treatment of individuals who are heterozygous carriers of a deleterious mutation in NPR2 with neither parent having short stature is also contemplated. Further contemplated is treatment of individuals who are heterozygous for deleterious mutations in other growth plate genes with CNP to improve stature and/or enhance bone growth.

Exemplary mutations in NPR2 are disclosed in International Patent Publication WO 2021/055497, incorporated herein by reference.

NPPC's role in skeletal growth is well documented (Hisado-Oliva et al., Genetics Medicine 20:91-97, 2018). The NPPC knock out mouse showed severe disproportionate form of dwarfism including shortening of limbs and endochondral ossification (Hisado-Oliva et al., 2018, supra). Human genome wide studies have shown a link between NPPC and height (Hisado-Oliva et al., 2018, supra). Although CNP haploinsufficiency has been believed to be a cause of short stature in humans, a recent study identified heterozygous mutations in families with short stature and hands (Hisado-Oliva et al., 2018, supra). These studies observed significant reduction in cGMP production as measured in heterozygous state (Hisado-Oliva et al., 2018, supra). Mutations in NPPC include a 355G>T missense mutation causing a Glyl19Cys change and a 349C>G missense mutation causing a Arg117Gly change. A CNP variant rescuing CGMP production may provide therapeutic benefit in the management of a disorder in patients having heterozygous loss-of-function NPPC mutations.

Leri-Weill dyschondrosteosis (LWD) is a rare genetic disorder characterized by shortening of the forearms and lower legs, abnormal misalignment of the wrist (Madelung deformity of the wrist), and associated short stature. LWD is caused by a heterozygous mutation in the short stature homeobox-containing (SHOX) gene or its regulatory elements located on the pseudoautosomal region 1 (PAR1) of the sex chromosomes. (See the Rare Disease Database and Carmona et al., Hum Mol Genet 20:1547-1559, 2011). The disorder Langer mesomelic dysplasia arises when there are two SHOX mutations, and may result from a mutation on each chromosome, either a homozygous or compound heterozygous mutations. A subset of SHOX mutations give rise to idiopathic short stature. Turner syndrome results due to a deletion on the X chromosome that can include the SHOX gene. SHOX has been identified as involved in the regulation of FGFR3 transcription and contributes to control of bone growth (Marchini et al., Endocr Rev. 37:417-448, 2016). SHOX deficiency leads to increased FGFR3 signaling, and there is some evidence to support that SHOX has direct interactions with CNP/NPR2 as well (Marchini, supra). Given the association of SHOX with FGFR3 and bone growth, it is contemplated that a subject having a homozygous or heterozygous SHOX mutation would benefit from treatment with CNP variants as described herein.

RASopathies are a group of rare genetic conditions caused by mutations in genes of the Ras/mitogen-activated protein kinase (MAPK) pathway. RASopathies are a group of disorders characterized by increased signaling through RAS/MAPK pathway. This pathway leads to downstream activation of the RAF/MEK/ERK pathway. Short stature is a characteristic feature of certain RASopathies. For example, CNP signaling inhibits RAF and leads to decreased MEK and ERK activation.

Treatment of RASopathies are contemplated herein. RASopathies associated with short stature include Noonan syndrome, Costello syndrome, Cardiofaciocutaneous syndrome, Neurofibromatosis Type 1, and LEOPARD syndrome. Hereditary gingival fibromatosis type 1 is also a RASopathy contemplated herein. RASopathy patients (including Noonan syndrome, Costello syndrome, Cardiofaciocutaneous syndrome, Neurofibromatosis Type 1, LEOPARD syndrome, hereditary gingival fibromatosis type 1) include patients with heterozygous variants in one or more of the following genes: BRAF, CBL, HRAS, KRAS, LZTR1, MAP2K1, MAP2K2, MRAS, NF1, NRAS, PPP1CB, PTPN11, RAF1, RRAS, RIT1, SHOC2, SOS1, or SOS2 (Tajan et al. Endocr. Rev. 2018; 39 (5): 676-700).

CFC is caused by mutations in several genes in the Ras/MAPK signaling pathway, including K-Ras, B-Raf, Mek1 and Mek2. Costello syndrome, also called faciocutaneoskeletal (FCS) syndrome is caused by activating mutations in the H-Ras gene. Hereditary gingival fibromatosis type I (HGF) is caused by dominant mutations in the SOS1 gene (Son of Sevenless homolog 1), which encodes a guanine nucleotide exchange factor (SOS) that acts on the Ras subfamily of small GTPases. Neurofibromatosis type I (NF1) is caused by mutations in the neurofibromin 1 gene, which encodes a negative regulator of the Ras/MAPK signaling pathway. Noonan syndrome (NS) is caused by mutations in one of several genes, including PTPN11, which encodes SHP2, and SOS1, as well as K-Ras and Raf-1.

CNP has been demonstrated to be an effective therapy in RASopathy models. Ono et al. generated mice deficient in Nf1 in type II collagen producing cells (Ono et al., Hum. Mol. Genet. 2013; 22 (15): 3048-62). These mice demonstrated constitutive ERK1/2 activation, and decreased chondrocyte proliferation, and maturation. Daily injections of CNP in these mice led to decreased ERK phosphorylation and corrected the short stature. A mouse model of Cardiofaciocutaneous syndrome using a Braf mutation (p.Q241R) (Inoue et al. Hum. Mol. Genet. 2019; 28 (1): 74-83). exhibited decreased body length and reduced growth plate width with smaller proliferative and hypertrophic zones compared to wild type, and CNP administration led to increases in body length in these animals.

Mutations in multiple genes can cause Noonan syndrome, which is characterized by short stature, heart defects, bleeding problems, and skeletal malformations. Mutations in the PTPN11 gene cause about half of all cases of Noonan's syndrome. SOS1 gene mutations cause an additional 10 to 15 percent, and RAF1 and RIT1 genes each account for about 5 percent of cases. Mutations in other genes each account for a small number of cases. The cause of Noonan syndrome in 15 to 20 percent of people with this disorder is unknown.

The PTPN11, SOS1, RAF1, and RIT1 genes all encode for proteins that are important in the RAS/MAPK cell signaling pathway, which is needed for cell division and growth (proliferation), differentiation, and cell migration. Many of the mutations in the genes associated with Noonan syndrome cause the resulting protein to be turned on (active) and this prolonged activation alters normal RAS/MAPK signaling, which disrupts the regulation of cell growth and division, leading to the characteristic features of Noonan syndrome. See, e.g., Chen et al., Proc Natl Acad Sci USA. 111 (31): 11473-8, 2014, Romano et al., Pediatrics. 126 (4): 746-59, 2010, and Milosavljevic et al., Am J Med Genet 170 (7): 1874-80, 2016. It is contemplated that a subject having mutations that activate the MAPK pathway would benefit from treatment with CNP variants as described herein to improve bone growth and short stature. It is also contemplated that a subject having mutations that activate the MAPK pathway would benefit from treatment with CNP variants as described herein to improve other comorbidities associated with an overactive MAPK pathway in other cells throughout the body where the NPR2 receptor is expressed on its surface.

Mutations in the PTPN11 gene, which encodes the non-receptor protein tyrosine phosphatase SHP-2, lead to disorders characterized by short stature such as Noonan's Syndrome (Musente et al., Eur J Hum Genet 11:201-206 (2003). Musente (supra) identifies numerous mutations in the PTPN11 gene that lead to short stature. Gain of function mutations lead to overactive signaling through SHP2 and inhibit Growth Hormone-induced IGF-1 release, thereby contributing to a decrease in bone growth (Rocca Serra-Nédélec, PNAS 109:4257-4262, 2012). It is contemplated that a subject having a homozygous or heterozygous PTPN11 mutation would benefit from treatment with CNP variants as described herein to improve bone growth and short stature.

Mutations in the Indian hedgehog (IHH) gene, which is related to regulation of endochondral ossification, have also been associated with short stature syndromes (Vasques et al., J Clin Endocrinol Metab. 103:604-614, 2018). Many IHH mutations identified segregate with short stature in a dominant inheritance pattern. Given the association of IHH with bone growth and ossification, it is contemplated that subjects having a homozygous or heterozygous IHH mutation will benefit from treatment with a CNP variant as described herein.

Mutations in FGFR3, including N540K and K650N, lead to short stature and hypochondroplasia.

Insulin-like growth factor 1 receptor (IGF1R) is a heterotetrameric (α2ß2) transmembrane glycoprotein with an intrinsic kinase activity. IGF1R has been shown to have a role in prenatal and postnatal growth. Heterozygous mutations in IGF1R have been identified in Small for gestational age children (SGA) and individuals with familial short stature (Kawashima et al., Endocrine J. 59:179-185, 2012). Mutations in IGF1R associated with short stature include R108Q/K115N, R59T, R709Q, G1050K, R481Q, V599E, and G1125A (Kawashima, supra).

Height is a highly heritable trait that can be influenced by the combined effect of hundreds or thousands of genes (Wood et al, 2014, Nature Genetics, 46:1173-1189). Short stature in an individual can be the result of the combined effect of these genes, without a single gene being the primary contributor. It is contemplated that such individuals with short stature defined by a height SDS of less than −1.0, −1.5, −2.0, −2.5, or −3.0, can be beneficially treated with a CNP variant given the ability of CNP to increase the length of normal animals, for example, enhance bone growth and length of bones.

In various embodiments, the CNP variants are useful to treat a subject with short stature having a height SDS of less than −1.0, −1.5, −2.0, −2.5, or −3.0, and having at least one parent with a height SDS of less than −1.0, −1.5, −2.0 or −2.5, optionally wherein the second parent has height within the normal range. In various embodiments, the CNP variants are useful to treat a subject with short stature having a height SDS of between −2.0 to −3.0. In various embodiments, the CNP variants are useful to treat a subject with short stature having a height SDS of between −2.0 to −2.5. In various embodiments, the short stature is associated with one or more mutations in a gene associated with short stature, such as, collagen (COL2A1, COL11A1, COL9A2, COL10), aggrecan (ACAN), indian hedgehog (IHH), PTPN11, NPR2, NPPC, FGFR3, or insulin growth factor 1 receptor (IGF1R), DTL, PAPPA2, or combinations thereof.

In various embodiments, the growth plate disorder or short stature is associated with one or more mutations in a gene associated with a RASopathy.

In various embodiments, the short stature is a result of mutations in multiple genes as determined by polygenic risk score (PRS). Polygenic risk scores (PRS) are calculated for height using the largest published genome-wide association study (GWAS) meta-analysis for height that do not include any samples from the UK Biobank project as described in WO 2021/055497. The cohort may be divided into five PRS quintiles (PRS 1 being the lowest height, PRS 5 the tallest height). In various embodiments, the subject has a mutation in NPR2 and a low PRS. In various embodiments, the subject has a mutation in FGFR3 and a low PRS. In various embodiments, the subject has a mutation in NPR2 and a low PRS. In various embodiments, the subject has a mutation in IGF1R and a low PRS. In various embodiments, the subject has a mutation in NPPC and a low PRS. In various embodiments, the subject has a mutation in SHOX and a low PRS. In various embodiments, the subject has one or more mutation in one or more of FGFR3, IGF1R, NPPC, NPR2 and SHOX, and a low PRS. In various embodiments, the PRS is 1 or 2. In various embodiments, the PRS is 1. In various embodiments, the PRS is 2.

In addition, the CNP variants are useful for treating other bone-related conditions and disorders, such as rickets, hypophosphatemic rickets [including X-linked hypophosphatemic rickets (also called vitamin D-resistant rickets) and autosomal dominant hypophosphatemic rickets], and osteomalacia [including tumor-induced osteomalacia (also called oncogenic osteomalacia or oncogenic hypophosphatemic osteomalacia)].

Disclosed herein is a method of treating a subject having a bone-related disorder, skeletal dysplasia or short stature described herein comprising,
i) identifying whether a subject has a Loss of Function (LoF) or Gain of Function (GoF) variant of a gene related to short stature;
ii) calculating a polygenic risk score (PRS) of the subject;
iii) determining if the subject has a LoF variant and a PRS in the bottom 20%; and
iv) treating the subject with a CNP variant if the subject has a LoF variant and a PRS in the bottom 20%.

In various embodiments, the subject has a PRS in the bottom 20%, 19%, 18%, 17.5%, 17%, 16.5%, 16%, 15.5%, 15%, 14.5%, 14%, 13.5%, 13%, 12.5%, 12%, 11%, 10%, 9%, 8%, 7.5%, 7%, 6%, 5%, 4%, 3%, 2.5%, 2% or 1%. In various embodiments, step iii) and iv) is a subject with a CNP variant if the subject has a LoF variant and a PRS in the bottom 12.5%.

Exemplary genes related to skeletal dysplasia or short stature, include but are not limited to, NPR2, SHOX, PTPN11, COL2A1, COL11A1, COL9A2, COL10), aggrecan (ACAN), indian hedgehog (IHH), NPPC, FGFR3, IGF1R, DTL, and pregnancy-associated plasma protein A2 (PAPPA2).

The existence of a LoF or GoF variant in a gene related to short stature is determined by a biological activity assay. In various embodiments, a LoF or GoF variant may be predicted based on mapping to the predicted 3D structure and activity domain of a protein encoded by the gene, e.g., using AlphaForm 3D mapping or other protein mapping tools.

In an exemplary method, the PRS is calculated by a genome-wide association study (GWAS) of height.

In certain embodiments, the CNP variants and compositions and formulations comprising the same of the present disclosure are useful for improving one or more of the symptom(s) or physiological consequences of a skeletal dysplasia, wherein the improvement may be increased absolute growth, increased growth velocity, increased qualitative computed tomography (QCT) bone mineral density, improvement in growth plate morphology, increased long bone growth, improvement in spinal morphology, improved elbow joint range of motion and/or decreased sleep apnea. In this regard, it is noted that the terms “improved”, “improvement”, “increase”, “decrease” and grammatical equivalents thereof are all relative terms that when used in relation to a symptom or physiological consequence of a disease state, refer to the state of the symptom or physiological consequence of the disease after treatment with a CNP variant (or composition or formulation comprising the same) of the present invention as compared to the same symptom or physiological consequence of the disease before treatment with a CNP variant (or composition or formulation comprising the same) of the present invention (i.e., as compared to “baseline”). As described above, a “baseline” state can be determined either through measurement of the state in the subject prior to treatment (which can subsequently be compared to the state in the same subject after treatment), or through measurement of that state in a population of subjects suffering from the same affliction that share the same or similar characteristics (e.g., age, sex and/or disease state or progression).

Also provided is a method of overcoming cell growth arrest induced by a constitutively active mutant fibroblast growth factor receptor 3 (FGFR-3) comprising contacting a cell expressing the constitutively active FGFR-3 with a CNP variant or a composition as described herein.

In yet another embodiment, the disclosure provides CNP variants that in vitro or in vivo stimulate the production of at least about 50%, 60%, 70%, 80%, 90%, 100%, 110%, 120%, 130%, 140% or 150% of the cGMP level produced under the same concentration of wtCNP22 (e.g., 1 uM). In a still further embodiment, the CNP variants of the disclosure in vitro or in vivo stimulate the production of at least about 50%, 60%, 70%, 80%, 90%, 100%, 110%, 120%, 130%, 140% or 150% of the cGMP level produced under the same concentration of wtCNP22 (e.g., 1 uM).

The disclosure also contemplates that modulation of treatment with CNP as described herein enhances or increases growth in the range of 25%-50% change from baseline in the subject. In one embodiment, an enhancement or increase in growth velocity is an increase in annualized growth velocity of at least about 25%, more preferably at least about 40%, change from baseline in the subject.

It is contemplated that any of the CNP variants, including conjugates, salts or prodrugs thereof, described herein are useful in the methods.

In various embodiments, the CNP variant is PGQEHPNARKYKGANKKGLSKGCFGLKLDRIGSMSGLGC (Pro-Gly-CNP-37) (SEQ ID NO: 1). In various embodiments, the peptide further comprises an acetyl group. In various embodiments, the acetyl group is on the N-terminus of the peptide. In various embodiments, the peptide further comprises an OH or an NH₂ group at the C-terminus. In various embodiments, the variant comprises one or more linker groups as described herein. In various embodiments, the linker is a hydrolysable linker, e.g., as described herein.

In various embodiments, the CNP variant is selected from the group consisting of

(SEQ ID NO: 47)
PGQEHPNARKYKGANKKGLSKGCFGLKLDRIGSMSGLGC;
(SEQ ID NO: 46)
PGQEHPQARRYRGAQRRGLSRGCFGLKLDRIGSMSGLGC;
(SEQ ID NO: 48)
PGQEHPNARRYRGANRRGLSRGCFGLKLDRIGSMSGLGC;
(SEQ ID NO: 49)
PGQEHPQARKYKGAQKKGLSKGCFGLKLDRIGSMSGLGC
[CNP-37(M32N); SEQ ID NO: 41]
QEHPNARKYKGANKKGLSKGCFGLKLDRIGSNSGLGC;
(Met-CNP-37; SEQ ID NO: 43)
MQEHPNARKYKGANKKGLSKGCFGLKLDRIGSMSGLGC;
(Pro-CNP-37; SEQ ID NO: 42)
PQEHPNARKYKGANKKGLSKGCFGLKLDRIGSMSGLGC;
[Gly-CNP-37 (M32N); SEQ ID NO: 44]
GQEHPNARKYKGANKKGLSKGCFGLKLDRIGSNSGLGC;
(Met-Gly-CNP-37; SEQ ID NO: 45)
MGQEHPNARKYKGANKKGLSKGCFGLKLDRIGSMSGLGC;
(Gly-CNP-37: SEQ ID NO: 2)
GQEHPNARKYKGANKKGLSKGCFGLKLDRIGSMSGLGC
(SEQ ID NO: 50)
GQEHPNARKYKGANPKGLSKGCFGLKLDRIGSMSGLGC;
(SEQ ID NO: 51)
GQEHPNARKYKGANQKGLSKGCFGLKLDRIGSMSGLGC;
(SEQ ID NO: 52)
GQEHPNARKYKGANQQGLSKGCFGLKLDRIGSMSGLGC;
(SEQ ID NO: 53)
GQEHPNARKYKGANKPGLSKGCFGLKLDRIGSMSGLGC;
and
(CNP-38)
(SEQ ID NO: 21)
LQEHPNARKYKGANKKGLSKGCFGLKLDRIGSMSGLGC, wherein

the CNP variant comprises a conjugate moiety. In various embodiments, the conjugate moiety is a synthetic polymeric group.

In various embodiments, the variant comprises a synthetic polymeric group coupled to the variant through a hydrolysable linker. In various embodiments, the synthetic polymeric group comprises a hydrophilic polymer moiety. In various embodiments, the hydrophilic polymer moiety comprises polyethylene glycol (PEG). In various embodiments, the hydrophilic polymer moiety comprises polyethylene glycol (PEG) having a 6 to 20 atom chain length. In various embodiments, the conjugate moiety comprises one or more acid moieties linked to a hydrophilic spacer as described herein.

In various embodiments, the conjugate moiety comprises one or more acid moieties linked to a hydrophilic spacer. In various embodiments, the hydrophilic spacer is any amino acid. In various embodiments, the hydrophilic spacer is gamma glutamic acid (γGlu). In various embodiments, the hydrophilic spacer is OEG (8-amino-3,6-dioxaoctanoic acid). In various embodiments, the hydrophilic spacer is gamma glutamic acid (γGlu) or OEG (8-amino-3,6-dioxaoctanoic acid). In various embodiments, the hydrophilic spacer is gamma glutamic acid (YGlu) linked to one or two or more OEG (8-amino-3,6-dioxaoctanoic acid). In various embodiments, the acid moiety is a fatty acid. Exemplary fatty acids include short chain, medium chain, or long chain fatty acids, or a dicarboxylic fatty acid. In various embodiments, the fatty acid is saturated or unsaturated. Contemplated are C-6 to C-20 fatty acids, including but not limited to, C-6, C-8, C-10, C-12, C-14, C-16, C-18 or C-20 fatty acids, saturated or unsaturated. In various embodiments, the fatty acid is decanoic acid, dodecanoic acid, myristic acid, palmitic acid, stearic acid, arachidic acid, or diacids of the same.

In various embodiments, the variant comprises one or more linker groups. In various embodiments, the linker is on a residue of the CNP cyclic domain or at a site other than the CNP cyclic domain. In various embodiments, the linker is on a lysine residue.

Efficacy of treatment is measured by various parameters. In various embodiments, efficacy is assessed as the change in annualized growth velocity from the baseline period to the intervention period. Efficacy will also be assessed as the change in height SDS from baseline to end of treatment as measured using the CDC growth curves, and growth velocity SDS will be based on the Bone Mineral Density in Childhood Study (Kelly et al., J. Clin. Endocrinol. Metab. 2014; 99 (6): 2104-2112).

Efficacy can also be measured using analysis of skull and brain morphology, e.g., using magnetic resonance imaging (MRI). At birth, children with achondroplasia have abnormalities of the base of the skull and boundaries of the foramen magnum due to defective endochondral ossification, resulting in stenosis of the foramen magnum and compression of the vital neural and vascular structures passing through it. Foramen magnum stenosis has been implicated as the major underlying cause of an increased risk for sudden deaths observed in children less than age 5 years with achondroplasia (Pauli et al., J Pediatr 1984; 104:342-8; Hashmi et al., Am J Med Genet A 2018; 176:2359-64). Skull and brain morphology analysis include measurement of improvements in facial volume, sinus volume, and foramen magnum area in patients, e.g., younger patients less than 6 months old, treated with CNP variants.

Provided herein is a method for increasing facial volume, facial sinus volume, and foramen magnum area in a subject 6 months old or less having a bone-related disorder, skeletal dysplasia or short stature comprising administering CNP variants, conjugates, salts or prodrugs thereof at a dose of at least 30 μg/kg. Also provided is a method of decreasing the incidence of sudden infant death, sleep disordered breathing, and necessity for neurosurgical decompression of the foramen magnum in a subject 6 months old or less having a bone-related disorder, skeletal dysplasia or short stature comprising administering CNP variants, conjugates, salts or prodrugs thereof at a dose of at least 30 μg/kg. In various embodiments, the CNP variant is administered at a dose of 30 μg/kg for 3 months, 6 months, 1 year or more. In various embodiments, the dose of CNP variant is decreased to 15 μg/kg when the subject is about 2 years old.

Change in facial volume, facial sinus volume, and foramen magnum area are measured by magnetic resonance imaging (MRI), and can be compared to baseline levels, healthy control subjects or untreated control subjects.

QoLISSY, the Quality of Life in Short Stature Youth, is assessed as directed (Quality of Life in Short Stature Youth-The QoLISSY Questionnaire User's Manual. Lengerich: Pabst Science Publishers; 2013).

### Biomarkers

Biomarker refers to a detectable biological substance or moiety whose level is increased or decreased in association with a particular disease condition or treatment regimen. In the present disclosure, biomarkers may be measured before, during and/or after administration of a CNP variant as described herein. Exemplary bone- or cartilage-associated biomarkers include, but are not limited to, NTproCNP, N terminal fragment of collagen X (CXM), CNP, cGMP, propeptides of collagen type II and fragments thereof, collagen type II and fragments thereof, propeptides of collagen type I and fragments thereof, collagen type I and fragments thereof, osteocalcin, proliferating cell nuclear antigen (PCNA), aggrecan chondroitin sulfate, collagen X, and alkaline phosphatase. Cartilage- and bone-associated biomarkers can be measured in any appropriate biological sample, including but not limited to tissues, blood, serum, plasma, cerebrospinal fluid, synovial fluid and urine. In some embodiments, the biomarkers are measured in blood, plasma or serum from animals undergoing efficacy/pharmacodynamic in vivo studies and/or from the conditioned media of ex vivo studies.

NTproCNP is an amino-terminal propeptide (NTproCNP) of CNP that is released from cells at an equimolar ratio with CNP. The biologically active forms of CNP are found in plasma in low concentrations due to the quick clearance rate of the peptide. NTproCNP is not cleared via the same mechanism and it is found in the circulation at 20- to 50-fold higher concentration (Olney et al., Clin Endocrinol (Oxf). 2012, 77:416-422).

It is contemplated that assessment of the effects of CNP therapy as described herein on bone growth are measured in relation to NTproCNP levels. For example, NTproCNP levels are measured in a sample and doses of CNP altered or changed to bring NTproCNP levels within +/−2 SDS of the mean NTproCNP for the population. NTproCNP mean levels for different populations have been studied in the following publications, herein incorporated by reference: Olneyet al. (2015). C-type natriuretic peptide plasma levels are elevated in subjects with achondroplasia, hypochondroplasia, and thanatophoric dysplasia. J Clin Endocrinol Metab, 100 (2), E355-359; Prickett et al., (2013). Impact of age, phenotype and cardio-renal function on plasma C-type and B-type natriuretic peptide forms in an adult population. Clin Endocrinol (Oxf), 78 (5), 783-789; Espiner et al. (2018). Plasma C-Type Natriuretic Peptide: Emerging Applications in Disorders of Skeletal Growth. Horm Res Paediatr, 90 (6), 345-357; Olney et al. (2012). Amino-terminal propeptide of C-type natriuretic peptide (NTproCNP) predicts height velocity in healthy children. Clin Endocrinol (Oxf), 77 (3), 416-422; Olney et al., (2007). Amino-terminal propeptide of C-type natriuretic peptide and linear growth in children: effects of puberty, testosterone, and growth hormone. J Clin Endocrinol Metab, 92 (11), 4294-4298; and Olney et al. (2016). Dynamic response of C-type natriuretic peptide and its aminoterminal propeptide (NTproCNP) to growth hormone treatment in children with short stature. Clin Endocrinol (Oxf), 85 (4), 561-568.

For example, Olney 2016 shows that children between 6 to 10 years old with idiopathic short stature can have an average baseline NTproSDS of −0.6, ranging from −1.0 to 0.7. Olney 2012 reported NTproCNP levels in healthy children/adolescents during stages of growth. NTproCNP SDS can be calculated based on the average NTproCNP levels of the different age populations, and therefore +/−2 SDS from this mean can also be calculated. NTproCNP levels of subjects with achondroplasia or hypochondroplasia are described in Olney 2015, showing that children approximately 3 to 8 years old have an NTproSDS average of 1.4, ranging from 0.4 to 1.8, while hypochondroplasia subjects (age 6.6 to 11) have an average NTproCNP SDS of 1.9, ranging from 1.8 to 2.3. Methods for determining NTproSDS levels are described herein and in the publications above.

Collagen type X biomarker (CXM) is a degradation fragment of collagen type X, comprising intact trimeric noncollagenous 1 (NC1) domain of type X collagen. CXM is released by active growth plates and decreases in samples as subjects age. CXM levels have been correlated with growth velocity in children (Coghlan et al., Sci Transl Med 2017, 9 (419): eaan4669).

Bone-specific alkaline phosphatase (BSAP or BAP) is a bone growth biomarker produced by osteoblasts and osteoclasts in growth plates and mineralized bone. Changes in BSAP may reflect growth plate activity, bone growth, and/or bone remodeling activity.

N-terminal pro-peptide of type I procollagen (PINP) is a potential pharmacodynamic bone growth biomarker, released during production of type I collagen. Changes in PINP may reflect changes in growth plate activity, bone growth, and/or bone remodeling.

Cross-linked C-telopeptides of type II collagen (CTXII) is a potential pharmacodynamic bone growth biomarker released during degradation of type II collagen. Changes in CTXII may reflect changes in growth plate activity, bone growth, bone remodeling, and/or articular cartilage remodeling.

### Formulations

The disclosure provides pharmaceutical compositions, including modified release compositions, comprising a CNP variant described herein, and one or more pharmaceutically acceptable excipients, carriers and/or diluents. In certain embodiments, the compositions further comprise one or more other biologically active agents (e.g., inhibitors of proteases, receptor tyrosine kinases, and/or the clearance receptor NPR-C).

The disclosure provides for modified release compositions comprising a conjugate moiety as described herein. Modified-release compositions include those that deliver a drug with a delay after its administration (delayed-release dosage) or for a prolonged period of time (extended-release dosage). Various embodiments of a CNP peptide conjugate provided herein include modified-release compositions, such as extended release, sustained or controlled release, and delayed release. The term “extended release composition” refers to a composition formulated in a manner in order to make the active ingredient/drug available over an extended period of time following administration (US Pharmacopeia). Extended-release dosage include sustained-release (SR) or controlled-release (CR) forms in which. Sustained release maintains drug release over a sustained period but not necessarily at a constant rate, while CR maintains drug release over a sustained period at a nearly constant rate (Pharmaceutics: Drug Delivery and Targeting. Yvonne Perrie, Thomas Rades, Pharmaceutical Press, 2009). Delayed-release compositions or products are modified to delay release of the drug substance for some period of time after initial administration.

In various embodiments, the modified release composition is an extended release composition. In various embodiments, the modified release composition is a sustained release composition. In various embodiments the sustained or extended release compositions comprises a CNP pro-drug.

In various embodiments, the composition comprises an excipient, diluent or carrier. In various embodiments, the extended release composition comprises an excipient, diluent or carrier. In various embodiments, the excipient, diluent or carrier is a pharmaceutically acceptable excipient, diluent or carrier.

Non-limiting examples of excipients, carriers and diluents include vehicles, liquids, buffers, isotonicity agents, additives, stabilizers, preservatives, solubilizers, surfactants, emulsifiers, wetting agents, adjuvants, and so on. The compositions can contain liquids (e.g., water, ethanol); diluents of various buffer content (e.g., Tris-HCl, phosphate, acetate buffers, citrate buffers), pH and ionic strength; detergents and solubilizing agents (e.g., Polysorbate 20, Polysorbate 80); anti-oxidants (e.g., methionine, ascorbic acid, sodium metabisulfite); preservatives (e.g., Thimerosol, benzyl alcohol, m-cresol); and bulking substances (e.g., lactose, mannitol, sucrose). The use of excipients, diluents and carriers in the formulation of pharmaceutical compositions is known in the art; see, e.g., Remington's Pharmaceutical Sciences, 18^{th} Edition, pages 1435-1712, Mack Publishing Co. (Easton, Pennsylvania (1990)), which is incorporated herein by reference in its entirety.

For example, carriers include without limitation diluents, vehicles and adjuvants, as well as implant carriers, and inert, non-toxic solid or liquid fillers and encapsulating materials that do not react with the active ingredient(s). Non-limiting examples of carriers include phosphate buffered saline, physiological saline, water, and emulsions (e.g., oil/water emulsions). A carrier can be a solvent or dispersing medium containing, e.g., ethanol, a polyol (e.g., glycerol, propylene glycol, liquid polyethylene glycol, and the like), a vegetable oil, and mixtures thereof.

In some embodiments, the compositions are liquid formulations. In certain embodiments, the formulations comprise a CNP variant in a concentration range from about 0.1 mg/ml to about 20 mg/ml, or from about 0.5 mg/ml to about 20 mg/ml, or from about 1 mg/ml to about 20 mg/ml, or from about 0.1 mg/ml to about 10 mg/ml, or from about 0.5 mg/ml to about 10 mg/ml, or from about 0.5 to 5 mg/ml, or from about 0.5 to 3 mg/ml, or from about 1 mg/ml to about 10 mg/ml. In various embodiments, the CNP variant is in a concentration of 0.8 mg/ml to 2 mg/ml. In various embodiments, the CNP variant is at a concentration of 0.8 mg/ml. In various embodiments, the CNP variant is at a concentration of 2.0 mg/ml. In various embodiments, the CNP variant is reconstituted from a lyophilized powder.

In further embodiments, the compositions comprise a buffer solution or buffering agent to maintain the pH of a CNP-containing solution or suspension within a desired range. Non-limiting examples of buffer solutions include phosphate buffered saline, Tris buffered saline, and Hank's buffered saline. Buffering agents include without limitation sodium acetate, sodium phosphate, and sodium citrate. Mixtures of buffering agents can also be used. In certain embodiments, the buffering agent is acetic acid/acetate or citric acid/citrate. The amount of buffering agent suitable in a composition depends in part on the particular buffer used and the desired pH of the solution or suspension. In some embodiments, the buffering agent has a concentration of about 10 mM±5 mM. In certain embodiments, the pH of a composition is from about pH 3 to about pH 9, or from about pH 3 to about pH 7.5, or from about pH 3.5 to about pH 7, or from about pH 3.5 to about pH 6.5, or from about pH 4 to about pH 6, or from about pH 4 to about pH 5, or is at about pH 5.0±1.0. In various embodiments, the pH is about 5.0, 5.1, 5.2, 5.3, 5.4, 5.5, 5.6, 5.7, 5.8, 5.9 or 6.0. In various embodiments, the pH is 5.5.

In other embodiments, the compositions contain an isotonicity-adjusting agent to render the solution or suspension isotonic and more compatible for administration. Non-limiting examples of isotonicity agents include NaCl, dextrose, glucose, glycerin, sorbitol, xylitol, and ethanol. In certain embodiments, the isotonicity agent is NaCl. In certain embodiments, NaCl is in a concentration of about 160±20 mM, or about 140 mM±20 mM, or about 120±20 mM, or about 100 mM±20 mM, or about 80 mM±20 mM, or about 60 mM±20 mM.

In yet other embodiments, the compositions comprise a preservative. Preservatives include, but are not limited to, m-cresol and benzyl alcohol. In certain embodiments, the preservative is in a concentration of about 0.4%+0.2%, or about 1%+0.5%, or about 1.5%+0.5%, or about 2.0%+0.5%.

In still other embodiments, the compositions contain an anti-adsorbent (e.g., to mitigate adsorption of a CNP variant to glass or plastic). Anti-adsorbents include without limitation benzyl alcohol, Polysorbate 20, and Polysorbate 80. In certain embodiments, the anti-adsorbent is in a concentration from about 0.001% to about 0.5%, or from about 0.01% to about 0.5%, or from about 0.1% to about 1%, or from about 0.5% to about 1%, or from about 0.5% to about 1.5%, or from about 0.5% to about 2%, or from about 1% to about 2%.

In additional embodiments, the compositions comprise a stabilizer. Non-limiting examples of stabilizers include glycerin, glycerol, thioglycerol, methionine, and ascorbic acid and salts thereof. In some embodiments, when the stabilizer is thioglycerol or ascorbic acid or a salt thereof, the stabilizer is in a concentration from about 0.1% to about 1%. In other embodiments, when the stabilizer is methionine, the stabilizer is in a concentration from about 0.01% to about 0.5%, or from about 0.01% to about 0.2%. In still other embodiments, when the stabilizer is glycerin, the stabilizer is in a concentration from about 5% to about 100% (neat).

In further embodiments, the compositions contain an antioxidant. Exemplary anti-oxidants include without limitation methionine and ascorbic acid. In certain embodiments, the molar ratio of antioxidant to CNP is from about 0.1:1 to about 15:1, or from about 1:1 to about 15:1, or from about 0.5:1 to about 10:1, or from about 1:1 to about 10:1 or from about 3:1 to about 10:1.

Pharmaceutically acceptable salts can be used in the compositions, including without limitation mineral acid salts (e.g., hydrochloride, hydrobromide, phosphate, sulfate), salts of organic acids (e.g., acetate, propionate, malonate, benzoate, mesylate, tosylate), and salts of amines (e.g., isopropylamine, trimethylamine, dicyclohexylamine, diethanolamine). A thorough discussion of pharmaceutically acceptable salts is found in Remington's Pharmaceutical Sciences, 18^{th} Edition, Mack Publishing Company, (Easton, Pennsylvania (1990)).

The pharmaceutical compositions can be administered in various forms, such as tablets, capsules, granules, powders, solutions, suspensions, emulsions, ointments, and transdermal patches. The dosage forms of the compositions can be tailored to the desired mode of administration of the compositions. For oral administration, the compositions can take the form of, e.g., a tablet or capsule (including softgel capsule), or can be, e.g., an aqueous or nonaqueous solution, suspension or syrup. Tablets and capsules for oral administration can include one or more commonly used excipients, diluents and carriers, such as mannitol, lactose, glucose, sucrose, starch, corn starch, sodium saccharin, talc, cellulose, magnesium carbonate, and lubricating agents (e.g., magnesium stearate, sodium stearyl fumarate). If desired, flavoring, coloring and/or sweetening agents can be added to the solid and liquid formulations. Other optional ingredients for oral formulations include without limitation preservatives, suspending agents, and thickening agents. Oral formulations can also have an enteric coating to protect the CNP variant from the acidic environment of the stomach. Methods of preparing solid and liquid dosage forms are known, or will be apparent, to those skilled in this art (see, e.g., Remington's Pharmaceutical Sciences, referenced above).

Formulations for parenteral administration can be prepared, e.g., as liquid solutions or suspensions, as solid forms suitable for solubilization or suspension in a liquid medium prior to injection, or as emulsions. For example, sterile injectable solutions and suspensions can be formulated according to techniques known in the art using suitable diluents, carriers, solvents (e.g., buffered aqueous solution, Ringer's solution, isotonic sodium chloride solution), dispersing agents, wetting agents, emulsifying agents, suspending agents, and the like. In addition, sterile fixed oils, fatty esters, polyols and/or other inactive ingredients can be used. As further examples, formulations for parenteral administration include aqueous sterile injectable solutions, which can contain antioxidants, buffers, bacteriostats, and solutes that render the formulation isotonic with the blood of the intended recipient; and aqueous and nonaqueous sterile suspensions, which can contain suspending agents and thickening agents.

Exemplary CNP formulations are described in U.S. Pat. Nos. 9,907,834 and 10,646,550. Use of CNP formulations having a pH in the range from about 4 to about 6 is contemplated.

Compositions comprising a CNP variant can also be lyophilized formulations. In certain embodiments, the lyophilized formulations comprise a buffer and bulking agent, and optionally an antioxidant. Exemplary buffers include without limitation acetate buffers and citrate buffers. Exemplary bulking agents include without limitation mannitol, sucrose, dextran, lactose, trehalose, and povidone (PVP K24). In certain embodiments, mannitol is in an amount from about 3% to about 10%, or from about 4% to about 8%, or from about 4% to about 6%. In certain embodiments, sucrose is in an amount from about 6% to about 20%, or from about 6% to about 15%, or from about 8% to about 12%. Exemplary anti-oxidants include, but are not limited to, methionine and ascorbic acid.

In various embodiments, the formulation comprises citric acid, sodium citrate, trehalose, mannitol, methionine, polysorbate 80, and optionally sterile water for injection (WFI).

The disclosure also provides kits containing, e.g., bottles, vials, ampoules, tubes, cartridges and/or syringes that comprise a liquid (e.g., sterile injectable) formulation or a solid (e.g., lyophilized) formulation. The kits can also contain pharmaceutically acceptable vehicles or carriers (e.g., solvents, solutions and/or buffers) for reconstituting a solid (e.g., lyophilized) formulation into a solution or suspension for administration (e.g., by injection), including without limitation reconstituting a lyophilized formulation in a syringe for injection or for diluting concentrate to a lower concentration. Furthermore, extemporaneous injection solutions and suspensions can be prepared from, e.g., sterile powder, granules, or tablets comprising a CNP-containing composition. The kits can also include dispensing devices, such as aerosol or injection dispensing devices, pen injectors, autoinjectors, needleless injectors, syringes, and/or needles.

As a non-limiting example, a kit can include syringes having a single chamber or dual chambers. For single-chamber syringes, the single chamber can contain a liquid CNP formulation ready for injection, or a solid (e.g., lyophilized) CNP formulation or a liquid formulation of a CNP variant in a relatively small amount of a suitable solvent system (e.g., glycerin) that can be reconstituted into a solution or suspension for injection. For dual-chamber syringes, one chamber can contain a pharmaceutically acceptable vehicle or carrier (e.g., solvent system, solution or buffer), and the other chamber can contain a solid (e.g., lyophilized) CNP formulation or a liquid formulation of a CNP variant in a relatively small amount of a suitable solvent system (e.g., glycerin) which can be reconstituted into a solution or suspension, using the vehicle or carrier from the first chamber, for injection.

As a further example, a kit can include one or more pen injector or autoinjector devices, and dual-chamber cartridges. One chamber of a cartridge can contain a pharmaceutically acceptable vehicle or carrier (e.g., solvent system, solution or buffer), and the other chamber can contain a solid (e.g., lyophilized) CNP formulation or a liquid formulation of a CNP variant in a relatively small amount of a suitable solvent system (e.g., glycerin) which can be reconstituted into a solution or suspension, using the vehicle or carrier from the first chamber, for injection. A cartridge can comprise an amount of the CNP variant that is sufficient for dosing over a desired time period (e.g., 1 day, 2 days, 3 days, 1 week, 2 weeks, 3 weeks, 4 weeks, etc.). The pen injector or autoinjector can be adjusted to administer a desired amount of the CNP formulation from a cartridge.

In addition, pharmaceutical compositions comprising a CNP variant can be formulated as a slow release, controlled release or sustained release system for maintaining a relatively constant level of dosage over a desired time period, such as 1 week, 2 weeks, 3 weeks, 1 month, 2 months, or 3 months. Slow release, controlled release and sustained release formulations can be prepared using, e.g., biodegradable polymeric systems {which can comprise, e.g., hydrophilic polymers [e.g., polylactide, polyglycolide, poly(lactide-glycolide)]}, and can take the form of, e.g., microparticles, microspheres or liposomes, as is known in the art.

### Administration and Dosing

As used herein, the term “therapeutically effective amount” of an active agent (e.g., a CNP variant) refers to an amount that provides therapeutic benefit to a patient. The amount may vary from one individual to another and may depend upon a number of factors, including the overall physical condition of the patient. A therapeutically effective amount of a CNP variant can be readily ascertained by one skilled in the art, using publicly available materials and procedures. For example, the amount of a CNP variant used for therapy should give an acceptable rate of reversal of cartilage degeneration or increase in cartilage growth.

The dosing frequency for a particular individual may vary depending upon various factors, including the disorder being treated and the condition and response of the individual to the therapy. In certain embodiments, a pharmaceutical composition containing a CNP variant is administered to a subject about one time per day, one time per two days, one time per three days, or one time per week, twice per week, three times per week, once every two weeks, or monthly.

The CNP variant compositions described herein can be administered to patients in need thereof at therapeutically effective doses to treat, ameliorate or prevent bone-related disorders and short stature disorders (e.g., skeletal dysplasias, including achondroplasia, hypochondroplasia, etc.). The CNP variants contemplated for use herein can be administered to patients at therapeutically effective doses to treat, ameliorate or prevent osteoarthritis and other conditions having an osteoarthritis-associated symptom. The safety and therapeutic efficacy of the CNP variants can be determined by standard pharmacological procedures in cell cultures or experimental animals, such as, for example, by determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Active agents exhibiting a large therapeutic index are normally preferred.

In certain embodiments, the CNP variant compositions described herein are administered at a dose in the range from about 3, 4, 5, 6, 7, 8, 9 or 10 nmol/kg to about 300 nmol/kg, or from about 20 nmol/kg to about 200 nmol/kg. In some embodiments, the CNP compositions are administered at a dose of about 3, 4, 5, 6, 7, 8, 9, 10, 15, 20, 25, 30, 35, 40, 45, 50, 55, 60, 65, 70, 75, 80, 85, 90, 95, 100, 110, 120, 125, 130, 140, 150, 160, 170, 175, 180, 190, 200, 210, 220, 230, 240, 250, 260, 270, 280, 290, 300, 350, 400, 450, 500, 750, 1000, 1250, 1500, 1750 or 2000 nmol/kg or other dose deemed appropriate by the treating physician. In other embodiments, the CNP variant compositions are administered at a dose of about 3, 4, 5, 6, 7, 8, 9, 10, 15, 20, 25, 30, 40, 50, 60, 70, 80, 90, 100, 110, 120, 130, 140, 150, 160, 170, 180, 190, 200, 250, 300, 350, 400, 450, 500, 550, 600, 650, 700, 750, 800, 850, 900, 950 or 1000 μg/kg, or about 0.5, 0.8, 1.0, 1.25, 1.5, 2, 2.5, 3, 3.5, 4, 4.5, 5, 5.5, 6, 6.5, 7, 7.5, 8, 8.5, 9, 9.5 or 10 mg/kg, or other dose deemed appropriate by the treating physician. The doses of CNP or CNP variant described herein can be administered according to the dosing frequency/frequency of administration described herein, including without limitation daily, 2 or 3 times per week, weekly, every 2 weeks, every 3 weeks, monthly, etc. In various embodiments, the CNP or CNP variant is administered daily subcutaneously. In various embodiments, the CNP or CNP variant is administered weekly subcutaneously. In various embodiments, the CNP variant is administered at a dose of 2.5 μg/kg/day to 60 μg/kg/day, 10 μg/kg/day to 45 μg/kg/day, or 15 μg/kg/day to 30 μg/kg/day. In various embodiments, the CNP variant is administered at a dose of 15 μg/kg/day. In various embodiments, the CNP variant is administered at a dose of 30 μg/kg/day.

The frequency of dosing/administration of a CNP variant for a particular subject may vary depending upon various factors, including the disorder being treated and the condition and response of the subject to the therapy. The CNP variant can be administered in a single dose or in multiple doses per dosing. In certain embodiments, the CNP variant composition is administered, in a single dose or in multiple doses, once daily, once weekly, once every two weeks, once every three weeks, once every 4 weeks, once every 6 weeks, once every two months, once every three months or once every six months, or as deemed appropriate by the treating physician. In various embodiments, the CNP variant is administered for 3 month, 6 months, 12 months or more.

In some embodiments, a CNP variant composition is administered so as to allow for periods of growth (e.g., chondrogenesis), followed by a recovery period (e.g., osteogenesis). For example, the CNP composition may be administered subcutaneously or by another mode daily or multiple times per week for a period of time, followed by a period of no treatment, then the cycle is repeated. In some embodiments, the initial period of treatment (e.g., administration of the CNP variant composition daily or multiple times per week) is for 3 days, 1 week, 2 weeks, 3 weeks, 4 weeks, 5 weeks, 6 weeks, 7 weeks, 8 weeks, 9 weeks, 10 weeks, 11 weeks or 12 weeks. In a related embodiment, the period of no treatment lasts for 3 days, 1 week, 2 weeks, 3 weeks or 4 weeks. In certain embodiments, the dosing regimen of the CNP variant compositions is daily for 3 days followed by 3 days off; or daily or multiple times per week for 1 week followed by 3 days or 1 week off; or daily or multiple times per week for 2 weeks followed by 1 or 2 weeks off; or daily or multiple times per week for 3 weeks followed by 1, 2 or 3 weeks off; or daily or multiple times per week for 4, 5, 6, 7, 8, 9, 10, 11 or 12 weeks followed by 1, 2, 3 or 4 weeks off.

The CNP variants, or pharmaceutical compositions comprising them, can be administered to subjects in various ways such as, e.g., by injection subcutaneously, intraarticularly, intravenously, intra-arterially, intraperitoneally, intramuscularly, intradermally, or intrathecally. In one embodiment, the CNP variants are administered by a single subcutaneous, intraarticular, intravenous, intra-arterial, intraperitoneal, intramuscular, intradermal, or intrathecal injection.

The CNP variants can be administered by implantation of a depot at the target site of action (e.g., an abnormal or degenerated joint or cartilage area). Alternatively, the CNP variants can be administered sublingually under the tongue (e.g., sublingual tablet) or by inhalation into the lungs (e.g., inhaler or aerosol spray), by delivery into the nasal cavity (e.g., intranasal spray), by delivery into the eye (e.g., eye drop), or by transdermal delivery (e.g., by means of a patch on the skin). The CNP variants may also be administered orally in the form of microspheres, microcapsules, liposomes (uncharged or charged (e.g., cationic)), polymeric microparticles (e.g., polyamides, polylactide, polyglycolide, poly (lactide-glycolide)), microemulsions, and the like.

A further method of administration is by osmotic pump (e.g., an Alzet pump) or mini-pump (e.g., an Alzet mini-osmotic pump), which allows for controlled, continuous and/or slow-release delivery of the CNP variant or pharmaceutical composition over a pre-determined period. The osmotic pump or mini-pump can be implanted subcutaneously, or near the target site (e.g., the long bones of limbs, the epiphyses, etc.).

It will be apparent to one skilled in the art that the CNP variants or compositions thereof can also be administered by other modes. Determination of the most effective mode of administration of the CNP variants or compositions thereof is within the skill of the skilled artisan.

The CNP variants can be administered as pharmaceutical formulations suitable for, e.g., oral (including buccal and sub-lingual), rectal, nasal, topical, pulmonary, vaginal or parenteral (including intramuscular, intraarterial, intrathecal, subcutaneous, intraarticularly and intravenous) administration, or in a form suitable for administration by inhalation or insufflation. Depending on the intended mode of administration, the pharmaceutical formulations can be in the form of solid, semi-solid or liquid dosage forms, such as tablets, suppositories, pills, capsules, powders, liquids, suspensions, emulsions, creams, ointments, lotions, and the like. The formulations can be provided in unit dosage form suitable for single administration of a precise dosage. The formulations comprise an effective amount of a CNP variant, and one or more pharmaceutically acceptable excipients, carriers and/or diluents, and optionally one or more other biologically active agents.

Additional aspects and details of the disclosure will be apparent from the following examples, which are intended to be illustrative rather than limiting.

### EXAMPLES

### Example 1—Measurement of NTproCNP

It was studied whether 1) daily dosing of Vosoritide—depending on dose and duration—inhibits endogenous CNP secretion during phases of increasing growth velocity (indirect feedback) and 2) whether endogenous CNP is unaffected by Vosoritide administered 4 hours previously (direct feedback).

In brief, 35 children (5-14 years of age) were enrolled in four sequential cohorts at nine study sites in this dose—finding and extension study. See e.g., Savarirayan, et al. N Engl J Med 2019, 381:25-35. Because measurements of plasma NTproCNP were available only in 28 of these subjects (age range 5-11, 12 male, 16 female), all data presented here apply to this subgroup alone. After screening at baseline, four separate cohorts-balanced by sex but distinguished by dose and timing of dose escalation-received daily subcutaneous injections of Vosoritide for periods up to 5.5 years. Cohort 1 (6 subjects, age range 6-10yr at screening) received 2.5 μg/kg/d for up to 10 months, followed by 7.5 μg/kg/d for approximately 2 months, and thereafter 15 μg/kg/d until study completion. Cohort 2 (6 subjects, age range 5-10) received 7.5 μg/kg/d for the initial 6-8 months-escalating to 15 μg/kg/d thereafter. Cohorts 3 (8 subjects, age range 6-11) and Cohort 4 (8 subjects, age range 5-8) received 15 μg/kg/d and 30 μg/kg/d respectively throughout the study. At completion of NTproCNP sampling, respective ages (means) were 13.2yr in Cohort 1, 13.7yr in Cohort 2, 13.6yr in Cohort 3 and 11.4yr in Cohort 4. Signs of pubertal development were observed after 2yr of treatment in all except in three subjects in Cohort 2, one subject in Cohort 3, and in two subjects in Cohort 4.

To assess possible correlations with changes in annualized growth velocity (AGV) and or during puberty, serial (pre injection) sampling was done throughout the study. EDTA anticoagulated plasma was collected at screening (baseline), then at 12 different time points during the 5 year period. Anticipating links between endogenous CNP and the initial phase of growth acceleration (Olney et al., Clin Endocrinol (Oxf) 2016, 85:561-568), sampling was more frequent in all cohorts during the initial 2 months of therapy. Measurements were also made on day 85 in Cohort 1, in three subjects in Cohort 2 but not other cohorts, so are not included in the analysis of serial changes in AGV. Sampling was less frequent after one year (at 1, at 2 year and then half yearly until final sampling at year 5.5).

To examine possible acute effects of Vosoritide on plasma NTproCNP, samples were drawn 4 hr after injection in all subjects on nine separate occasions across the first two years of the study. In Cohort 1 and 2 subjects, samples were drawn at both 4 hr and 8 hr after the first injection. All other samples were drawn 4 hr after the routine morning injection.

All plasma NTproCNP measurements were carried out in duplicate by the Christchurch laboratory assay using their previously determined age and sex adjusted normal ranges (Standard Deviation Scores, SDS) for normal children (Olney et al. Clin Endocrinol (Oxf) 2012, 77:416-422). There was no detectable cross-reactivity of CNP (1-37pro gly) in the NTproCNP assay. This assay has a detection limit of 1.5 pmol/l, and within and between assay coefficients of variation of 6 and 7% respectively at 18 pmol/l. In previous studies of 6 healthy young adults (unpublished) values showed no evidence of diurnal fluctuation or effect of food ingestion during 0900 h and 1500 hr (average coefficient of variation 6.4%)—and no significant variation in samples drawn at short intervals on successive days. Because both age and sex affect plasma concentrations of NTproCNP in normal children (Prickett, et al. Pediatr Res 2005 58:334-340) all measured concentrations of NTproCNP were converted to SDS using data from reference ranges previously determined from 258 normal healthy children aged 2 months to 20 yr (Olney et al. Clin Endocrinol (Oxf) 2012; 77:416-422). As SDS for plasma NTproCNP are not available for untreated Ach, the above approach was deemed to be the optimal comparator for use in these samples drawn from children of differing age and sex, because the tempo and pubertal timing of height velocity in untreated Ach does not seem to differ from those in normal non Ach children (Merker et al. Am J Med Genet A 2018; 176:1723-1734).

Baseline NTproCNP is elevated relative to the general population in subjects with Achondroplasia. Baseline values of plasma NTproCNP at screening were raised (mean SDS 0.66±0.17, P<0.001) despite significantly lower AGV (mean 3.9±0.3 cm/y) when compared to general population children (Kelly et al. J Clin Endocrinol Metab 2014, 99:2104-2112) of this age group as shown by AGV SDS. Relevant baseline data of the four cohorts relating to age, plasma NTproCNP, AGV and AGV SDS at screening are shown in Table 1 along with the increment in AGV after 6 months of therapy. NTproCNP SDS was lower in Cohort 1, and age was lower in Cohort 4. Cohorts 3 and 4 receiving higher doses of Vosoritide (15 and 30 μg/kg/day respectively) exhibited significant and similar increase in AGV as assessed at 6 months (P<0.05 for both).

TABLE 1
Age, plasma NTproCNP, SDS, Annualized Growth Velocity (AGV) and AGV-SDS
at screening and increase (delta) at 6 months after commencing therapy.

| | Age | NTproCNP | NTproCNP | AGV | | Delta AGV @ 6 |
|---|---|---|---|---|---|---|
| | (years) | (pmol/L) | SDS | (cm/y) | AGV SDS | months (cm/y) |
| Cohort 1 | 8.0 ± 0.6 | 36.2 ± 2.3 | 0.1 ± 0.3 | 3.6 ± 0.5 | −2.5 ± 0.4 | 0.0 ± 0.6 |
| Cohort 2 | 8.4 ± 1.0 | 45.4 ± 4.0 | 1.1 ± 0.4 | 3.2 ± 0.2 | −2.6 ± 0.6 | 0.5 ± 0.4 |
| Cohort 3 | 8.6 ± 0.6 | 43.5 ± 3.2 | 1.0 ± 0.3 | 4.0 ± 0.9 | −1.5 ± 0.8 | 2.1 ± 0.8 |
| Cohort 4 | 7.4 ± 0.3 | 43.6 ± 2.9 | 0.9 ± 0.3 | 4.2 ± 0.7 | −2.1 ± 0.7 | 2.5 ± 0.9 |

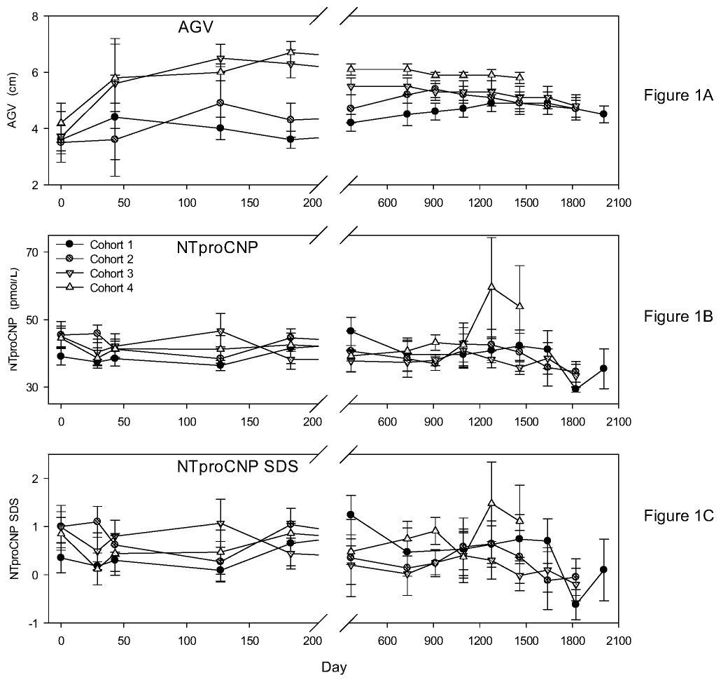

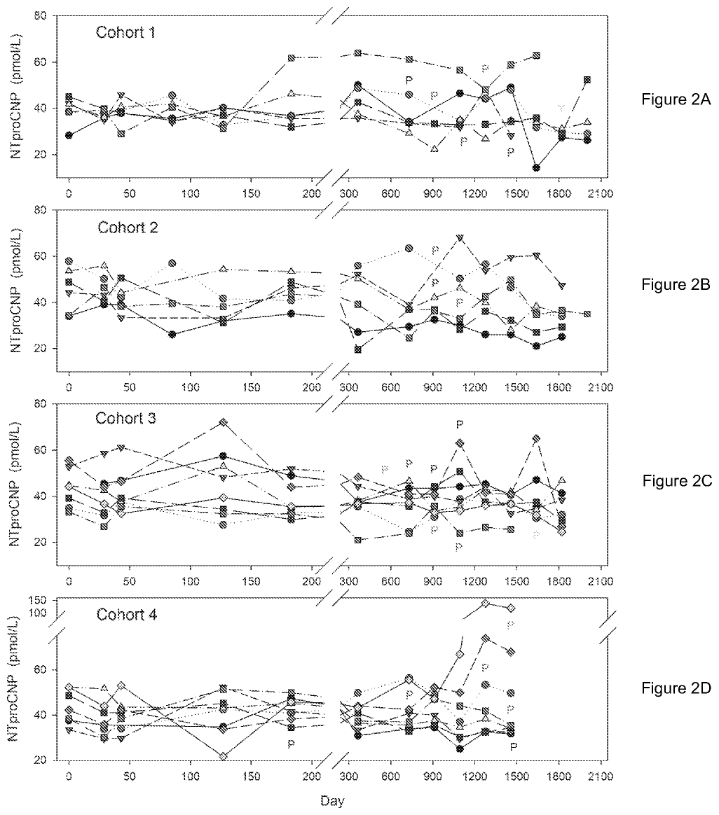

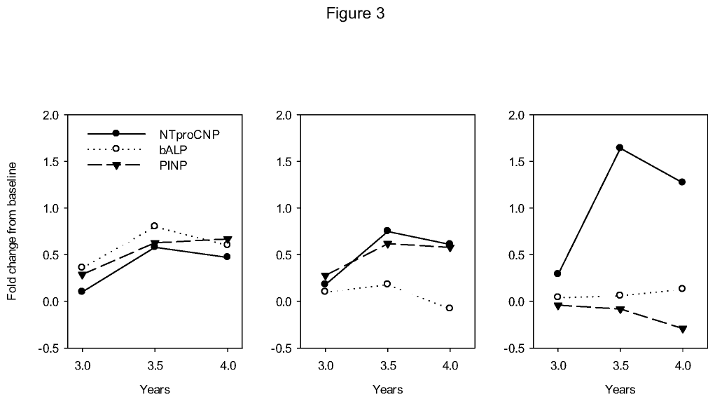

Sudden change in growth velocity is associated with a suppression of NTproCNP. Dynamic changes in NTproCNP and SDS in relation to growth promoting actions of Vosoritide are shown for each cohort in FIG. 1. Changes by individual per cohort are shown in FIG. 2. Sharp inflexions in AGV in Cohorts 3 and 4 across the initial 3 months were associated with significant fall in NTproCNP SDS at one month (P=0.04 and 0.004, cohort 3 and 4 respectively). At later time points in the first year of treatment of these two groups when AGV had stabilized, mean NTproCNP SDS was more variable but trended lower than baseline (FIG. 1). When the dose was escalated to 15 μg/kg/day after one year in cohorts 1 and 2, increases in AGV were associated with decline in NTproCNP (FIG. 1), but lack of frequent sampling early in the course of dose escalation prevents a more detailed analysis of temporal changes.

Anomalous increases in NTproCNP. Inspection of FIG. 1 shows relatively unchanging AGV from years 2-4 associated with unchanging NTproCNP excepting those in Cohorts 4. In Cohorts 2 and 3 (both receiving 15 μg/kg dose) mean NTproCNP SDS was lower than at screening at all-time points. In Cohorts 1 and 4, several anomalous and unexplained elevations were observed-all unassociated with change in AGV. Marked increases in plasma levels were observed after 3-4 years of treatment with 30 μg/kg/d (Cohort 4) in three of eight children (FIG. 2). Increases in SDS in these children (3.1, 6.7; 1.4, 3.0; and 0.3, 1.9—pre and peak respectively) were sustained for at least one year and were not consistently associated with changes in either BALP or PINP (FIG. 3). In two subjects, Tanner stage 2 pubertal development was documented but in the subject with the highest NTproCNP (138 pmol/L, SDS 6.7) puberty had not developed until breast budding was notated 6 months after the last measurement. Occasional spikes in plasma NTproCNP were observed in other subjects, some of which coincided with puberty staging (FIG. 2). In contrast, in one female child (Cohort 1) age 6.5 yr, plasma level increased abruptly some 6 months after starting the 2.5 μg/kg dose (FIG. 2). SDS (0.16 at screening) rose to 2.6 at 6 months and remained elevated for the remaining 4.5 yr but was unaffected by normal pubertal progression at age 10.5 yr. No consistent link was apparent with BALP, PINP or AGV.

Persistent growth under exogenous CNP effect was associated with persistent suppression of NTproCNP. To assess any impact of chronological age or duration of therapy on plasma NTproCNP, comparison was made between SDS at one year and at the study's completion. Since Cohorts 1-3 were all receiving 15 μg/kg/d dose after one year, values were combined. As shown in Table 2, significant decline in NTproCNP SDS was observed over time (F=14, p=0.002,) during periods of relatively unchanging AGV (FIG. 3).

TABLE 2
Effect of 15 μg/kg/day Vosoritide on mean NTproCNP
SDS after 1-5 years of therapy (n = 17)

| Study Year | NTproCNP SDS |
|---|---|
| 1.0 | 0.54 ± 0.28 |
| 4.0 | 0.32 ± 0.26 |
| 4.5 | 0.22 ± 0.29 |
| 5.0 | −0.20 ± 0.21 * |

* Significant difference from year 1 (P = 0.015)

Acute exposure to exogenous CNP analogue inhibits NTproCNP production. The impact of the first injection of Vosoritide was studied only in Cohorts 1 and 2. In Cohort 1, no change in NTproCNP 4 hr after injection was observed after 2.5 μg/kg/d-nor in any of the six studies undertaken in the subsequent 10 months of treatment. In Cohort 2, significant fall (pre 43.3±3.5, post 35±2.7 pmol/L, P=0.013, n=8) resulted from the initial 7.5 μg/k dose, returning to pre injection levels (44.3±4.7 pmol/L) at 8 hr post injection. In these same subjects, significant suppression was observed one month after commencing treatment (P=0.02) at which time AGV had not changed (Table 1). Across all subjects, results from paired samples were available on up to eight different time points during the initial 24 months of Vosoritide treatment. Since responses did not differ according to dose injected (F=0.8, P=0.5), results on any given time point were combined for statistical analysis.

TABLE 3
Change (delta) in plasma NTproCNP (pmol/L) at 4 hr
post Vosoritide in relation to duration of therapy

| Day | N* | Pre | Post | Delta | P value |
|---|---|---|---|---|---|
| 29 | 28 | 39.9 ± 1.5 | 37.5 ± 1.4 | −2.4 ± 1.5 | 0.12 |
| 43 | 25 | 40.6 ± 1.5 | 40.1 ± 1.6 | −0.5 ± 1.0 | 0.62 |
| 127 | 27 | 41.5 ± 2.0 | 39.8 ± 1.8 | −1.7 ± 1.5 | 0.27 |
| 183 | 28 | 42.4 ± 1.4 | 37.3 ± 1.3 | −5.0 ± 1.6 | 0.003 |
| 365 | 22 | 40.5 ± 2.2 | 36.2 ± 2.2 | −4.3 ± 1.4 | 0.006 |
| 730 | 17 | 39.6 ± 2.4 | 35.5 ± 2.4 | −4.2 ± 1.5 | 0.015 |

*Number of subjects

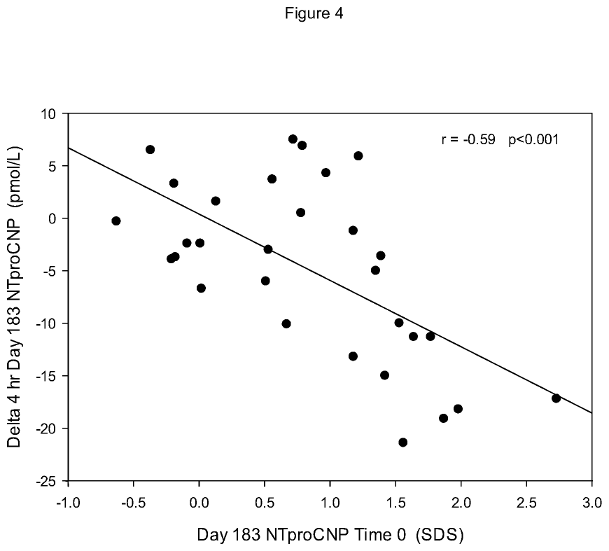

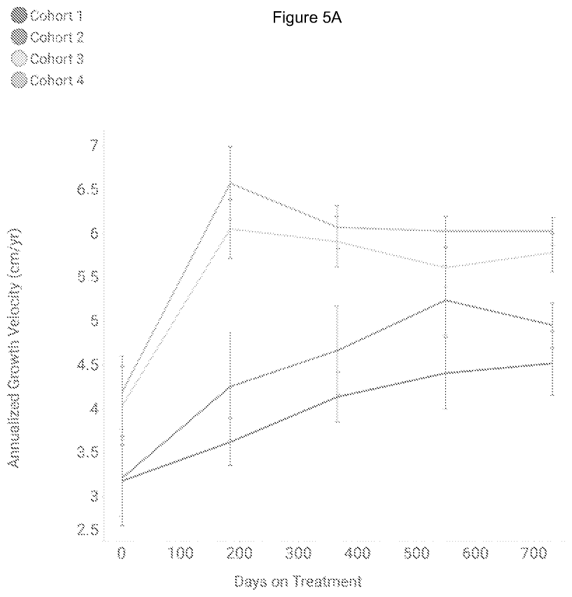

As shown in Table 3, when the grouped data was analyzed no significant change at 4 hr was found within the first 6 months of therapy (that is on days 29, 43 and 127). However, at each subsequent time point, a significant fall in plasma NTproCNP was observed on days 183 (P=0.003, n=30), 12 months (P=0.006, n=22) and at 24 months (P=0.015, n=17) (see Table 3). Irrespective of significance in the decline at 4 h itself, when links with concurrent NTproCNP SDS at the time of injection were examined, associations of fall in plasma NTproCNP (delta) with pre injection plasma NTproCNP SDS were identified at screening (r=−0.71, P<0.001); at Day 29 (r=−0.45, P=0.012); at Day 127 (r=−0.42, P=0.02); at Day 183 (r=−0.59, P<0.001, see FIG. 4) and at 24 months (r=−0.36, P=0.10). Higher pre-test SDS strongly associates with the fall in plasma NTproCNP at 4 hr. Together these results support a direct inhibitory effect of exogenous CNP on CNP production which is not observed during the initial 6 month period if skeletal growth accelerates.

Additionally, the data suggests NTproCNP SDS at baseline is significantly increased. Second, significant declines in plasma NTproCNP during the initial acceleration in AGV by growth-promoting doses of Vosoritide provides evidence of feedback regulation, which is likely to be indirect. Third, the decline in plasma NTproCNP 4 hr after injection of Vosoritide—in proportion to plasma NTproCNP SDS just prior to injection—is consistent with a direct feedback effect. These and other findings of marked increase in NTproCNP in early adolescence in some subjects receiving higher doses are important new observations that call for closer study.

Studies from rodent pups, growing lambs and children support the view that circulating levels of CNP products in plasma (CNP and NTproCNP) are sourced largely from growth plate or closely related tissues (Espiner 2018, supra). Measurement of any possible impact on endogenous proCNP production of administered CNP 1-37 Pro Gly (which strongly cross-reacts with CNP but not NTproCNP assay) is therefore only feasible using plasma concentrations of NTproCNP adjusted for age and sex (SDS). As previously reported (Olney et al., J Clin Endocrinol Metab 100: E355-359, (2015), it was confirmed that levels in healthy children with Ach are significantly increased prior to treatment- and remain so until the final phase of the study. Notably, during doses that sharply increased AGV within 2-3 months (15 and 30 μg/kg/d in Cohorts 3 and 4), NTproCNP SDS fell significantly, coinciding with the first signs of increments in serum collagen X marker, a degradation product of type X collagen. These changes were not observed in subjects receiving lower doses (2.5 and 7.5 μg/kg/d) where AGV was unaffected at this time. It is instructive to compare these dynamic changes in CNP with those found in short non Ach children of similar age starting daily doses of Human Growth Hormone (HGH) (Olney, et al. Clin Endocrinol (Oxf) 85:561-568, 2016). In that setting, similar inflections in AGV were associated with increase in NTproCNP (mean 11 pmol/L, delta 22% at day 21) in contrast to the concurrent fall of NTproCNP (approximately 6 pmol/L, range 3-11 pmol/L, delta-12%) at Day 29 during 15-30 μg/kg/d Vosoritide treatment in Cohorts 3 and 4. Disparate responses are unsurprising since growth plate concentrations of CNP products are clearly increased by growth hormone but not by exogenous CNP. These kinetic responses could relate to the estimated times (approximately 20-22 days) (Sansone et al., J Pediatr Orthop 29:61-67, 2009) for recruited chondrocytes to traverse the respective zones of the expanding growth plate and populate the primary spongiosa. However, unlike the persisting elevation in NTproCNP above baseline during the first year of HGH treatment (Olney, 2016, supra), the initial marked depressions in concentrations during Vosoritide therapy were less sustained-possibly reflecting the much shorter half-life of Vosoritide (28 minutes) compared to the much longer duration of HGH activity (up to 12 hr) (Olney, 2016, supra).

Taken together, the current findings are consistent with an indirect feedback mechanism whereby a factor generated by accelerated growth plate activity- or osteoid tissue—reduces local NPPC expression or secretion of proCNP into the extra cellular fluid- and align with the indirect negative feedback from exogenous CNP observed in 4 week old rodent pups 10 However in that study, 3 days of continuous high dose intravenous infusions of CNP 53, while significantly reducing lumbar vertebral nppc expression, did not reduce plasma NTproCNP in male rats (n=6) and were associated with a small decline in females (n=6). No indices of skeletal of growth plate activity in this brief exposure to CNP 53 were reported so possible links of accelerating endochondral bone growth with reduced plasma NTproCNP in this setting remain to be studied. Further study of larger groups of subjects and more appropriately timed sampling points, particularly within the initial 3 months of starting exogenous CNP therapy, can be expected to advance understanding of these dynamic changes in relation to changing bone growth in children, and may provide clinical applications. For example, decline in NTproCNP at one month- or targeting zero NTproCNP SDS in Ach-could be used to predict optimal effect size, duration of effect on growth plate activity and choice of dose and frequency of injections. Optimal effect size refers to a measure of the expected average normal growth rate based on population norms.

Surprisingly, in multiple studies undertaken at different times across the initial 2 years of treatment strong evidence for acute feedback was revealed but only after 6 months of exogenous therapy. This temporal constraint may relate to the early growth spurt observed in many of the children studied. Those in cohorts 3 and 4 exhibited initial sharp inflections in AGV-reducing plasma NTproCNP drawn 24 h post injection in this period-which may limit any decrease at 4 h from an injected dose. Neither group was tested prior to one month but no suppressive impact from either 15 or 30 μg dose was seen on day 29, 43, 127 or day 183 (when AVG was accelerating) but was clearly observed at later time points. AGV in the first 6 months was not significantly affected in either cohort 1 or cohort 2. In the former, the initial dose was insufficient to affect bioactivity (urine cGMP) on day 1 (Savarirayan, et al., N Engl J Med 381:25-35 2019), or consistently increase plasma CNP 39 within 2 h (Yasoda, supra 2004), and did not affect AGV. Lack of suppression at 4 h in any of the 8 studies conducted is therefore unsurprising. On the other hand, after 7.5 μg/kg/d (cohort 2) significant increase in urine cGMP and peak CNP 39 was observed on day 1 along with significant suppression of NTproCNP at 4 h—also observed in this cohort on day 29. Unfortunately, during the period of dose escalation in these 2 cohorts, sampling frequency was insufficient to assess any impact of accelerating AGV on NTproCNP—or the possible effect of this on the response at 4 h. While other factors such as increase in body weight, puberty, dose and bioavailability (much greater in cohort 3 and 4) (Savarirayan, supra) and the small number of subjects studied need to be considered, collectively the findings suggest that an interaction between indirect and direct feedback systems may account for the reduced impact of Vosoritide at 4 h in the first 6 months. Combining all groups, highly significant decline in NTproCNP at 4 h after dosing on days 183, 365 and 730 was found (Table 3). Notably, evaluating the response across all studies according to the NTproCNP SDS shows that the acute fall (effect size) is strongly dependent on the SDS at the time of dosing. This finding suggests that by restoring intra cellular CNP activity via a functional receptor (NPR2), CNP production is reduced in proportion to the prevailing level of resistance. Conceivably increased expression of the clearance receptor NPR3, reducing CNP and increasing NTproCNP (the antithesis of loss of function in NPR3 (Boudin et al., Am J Hum Genet 103:288-295, 2018) could account for the findings that were observed. However, since both CNP and NTproCNP are similarly increased in untreated children with Ach (Olney, supra) and the ratio NTproCNP/CNP is normal, upregulation of NPR3 is unlikely. Of note, no direct feedback was found in wild type rodent pups (Ueda, supra)-raising the possibility that the findings of suppression (which were not sex dependent) could be specific to Ach where circulating CNP products are elevated above normal. In clinical (safety) studies (BMN 111-101) undertaken in healthy normal adult males, no significant change in plasma NTproCNP from baseline was observed at 4 hr after doses ranging from 2.5-15 μg/kg/d. This suggests that direct suppression characterizes the immature skeleton but whether confined to Ach requires further study—for example children without the genetic disorder—as well as in Ach where the SDS is close to zero. Notwithstanding these findings, it is unlikely that the direct feedback observed contributes to CNP regulation in vivo considering the very high concentrations (>350 pmol/L peak) associated with significant suppression, and levels seen in pathophysiology (2-8 pmol/L) (Olney et al., J Clin Endocrinol Metab 100: E355-359, 2015).

### Example 2—Measurement of CXM and Other Biomarkers

Vosoritide acts on growth plate chondrocytes through the Natriuretic Peptide Receptor-B to stimulate increased endochondral bone growth, leading to increased growth velocity in treated subjects. In clinical studies, subject blood and urine samples were analyzed to monitor putative bone growth biomarkers including cross-linked C-terminal telopeptides of collagen II (CTxII), Bone-Specific Alkaline Phosphatase (BSAP), N-terminal pro-peptide of collagen I (PINP), and an N-terminal fragment of Collagen X (CXM). Changes in biomarkers over time were analyzed in relation to observed changes in growth velocity in subjects receiving vosoritide.

Collagen type X biomarker (CXM; Coghlan 2017) is a degradation fragment of collagen type X, released by active growth plates. A relative quantitative biomarker ECLA was developed and validated at BioMarin to measure CXM. Ninety-six-well Meso Scale Discovery (MSD) Streptavidin plats were blocked with StartingBlock PBS with Tween-20 (ThermoFisher Scientific, Waltham, MA, USA). After decanting blocking buffer, biotinylated anti-human collagen type X NC1 domain capture SOMAmer was incubated on the plate. The standard stock (recombinant human collagen type X NC1 domain in assay diluent [AD]) was serially diluted in AD, while the serum quality control samples (QC) and serum study samples were diluted 1:100 in AD. After washing the assay plate, diluted calibrators and samples were incubated on the plate. After a second wash, ruthenium-labeled mouse monoclonal anti-Collagen type X NC1 domain IgG detection antibody was incubated on the plate. The plate was then washed, MSD Read Buffer T with surfactant was added, and the plate was read on an MSD Quickplex instrument. The raw signal from each well was proportional to the collagen type X concentration in each sample. The concentration of collagen type X in each unknown sample was determined by interpolation of raw assay signal using the standard calibrator curve. The standard regression performed by Watson LIMS used a 4 Parameter Logistic (4-PL) Marquardt model with a weighting factor of 1/Y². The assay limit of detection was 914 μg/mL CXM in human serum.

An enzyme immunoassay method to measure bone-specific alkaline phosphatase in human serum was validated at ICON Labs (Farmingdale, NY, USA; validation N08-024VR-1,4). The assay used monoclonal anti-BAP antibody-coated wells to capture BAP in samples. The enzyme activity of the captured BAP was detected with p-Nitrophenyl phosphate substrate. Raw assay signals were read using a SpectraMax Spectrophotometer (Molecular Devices, San Jose, CA, USA). The concentration of BAP in each sample was determined by interpolation using the standard calibrator curve with a linear curve fit. The assay limit of quantification was 2 U/L in neat

A quantitative competitive format radioimmunoassay (RIA) method based on the UniQ PINP RIA assay kit (Orion Diagnostica, Espoo, Finland) was validated at ICON Labs (Farmingdale, NY, USA; validation N06-016VR). A known amount of ¹²⁵I-labelled PINP and an unknown amount of unlabeled PINP in samples competed for a limited number of high affinity binding sites on a polyclonal rabbit anti-PINP IgG antibody. A secondary anti-rabbit IgG antibody coated on solid kaolin particles was used to separate antibody-bound PINP from matrix components. The radioactivity of the bound ¹²⁵I-PINP was measured using a WIZARD automatic gamma counter (Perkin Elmer, Waltham, MA, USA). The amount of radioactivity in each tube was inversely proportional to the concentration of PINP in each sample. The concentration of PINP in each sample was determined by interpolation using a standard calibrator curve and a linear regression curve fit. The lower limit of quantitation was 5 μg/L PINP in neat human serum.

A quantitative competitive format ELISA for measurement of CTXII in human urine, using the CartiLaps ELISA kit from ImmunoDiagnostic Systems (East Boldon, UK) was validated at ICON Labs to support study 111-202/205 (validation N06-114VR). The assay was based on the competitive binding of a mouse monoclonal anti-CTXII antibody to urinary fragments of type II collagen or to biotinylated, synthetic peptides bound to the surface of microtiter plates coated with streptavidin. Initially, biotinylated, synthetic peptides were bound to the surface of streptavidincoated wells of the microtiter plate. After washing, standards, controls, and urine samples containing unlabeled CTXII were pipetted into the wells followed by addition of a solution of a mouse monoclonal anti-CTXII IgG. The wells were washed, and a solution of peroxidaseconjugated rabbit anti-mouse IgG was added to the wells. Following a second washing step, tetramethylbenzidine (TMB) chromogenic substrate was added to all wells. The yellow color development was stopped with sulfuric acid and the absorbance at 450 nm was read on a SpectraMax Plus spectrophotometer (Molecular Devices, San Jose, CA, USA). The raw signal in each well was inversely related to the concentration of CTXII in each sample. The concentration of CTXII in each sample was determined by interpolation using a standard calibrator curve and a linear curve fit. The lower limit of quantitation was 0.60 ng/mL CTXII in neat urine.

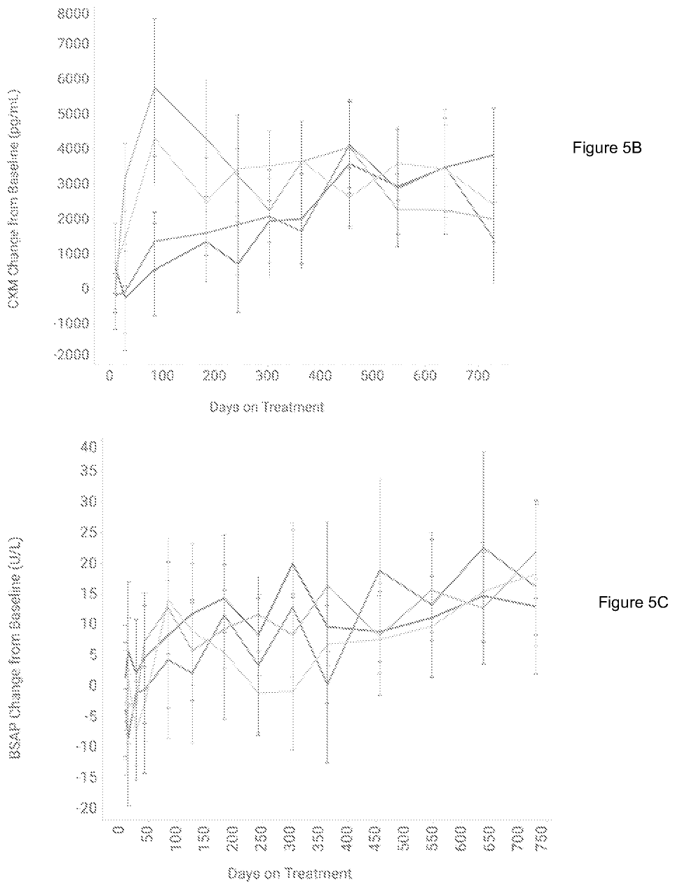

In study 111-202, pediatric subjects with achondroplasia age 5 to 15 received vosoritide at 2.5, 7.5, 15, or 30 μg/kg/day (cohorts 1, 2, 3, and 4, respectively) for the first 6 months. After 6 months, significant increases in annualized growth velocity (AGV) were observed in subjects receiving vosoritide at 15 or 30 μg/kg/day, but not for subjects receiving 2.5 or 7.5 g/kg/day (FIG. 5). After 6 months, the dose concentration for subjects in cohorts 1 and 2 was increased to 15 μg/kg/day, resulting in increased AGV.

While all of the biomarkers analyzed in 111-202 were variable, CXM demonstrated dose-dependent increases, BSAP appeared to increase slightly over time on treatment for all dose levels, and there was no apparent trend or dose dependent response for CTxII or P1NP. Based on these results, BSAP and CXM were incorporated into the placebo-controlled Phase III vosoritide clinical study 111-301 and pre-treatment natural history study 111-901.

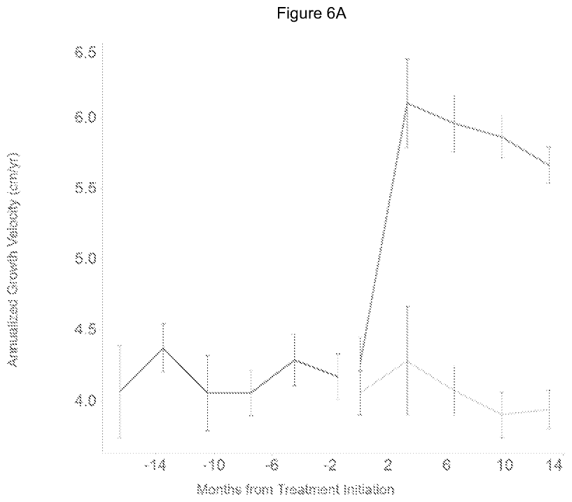

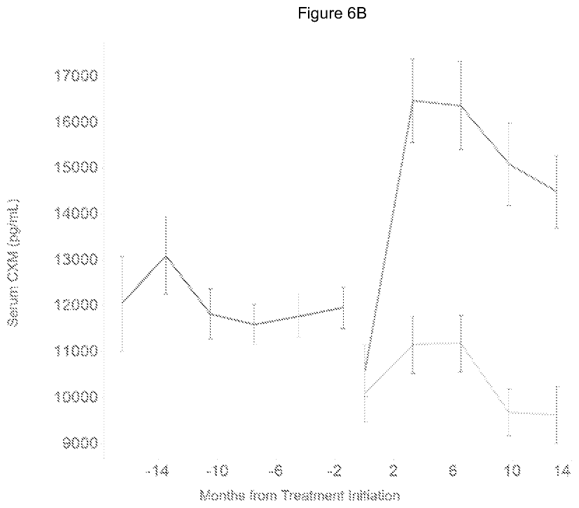

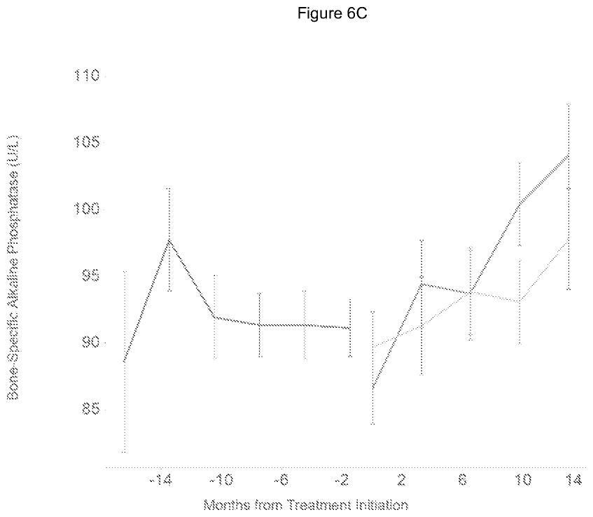

Before entering study 111-301, pediatric subjects with achondroplasia age 5 to 15 years old were monitored without treatment in study 111-901 for at least 6 and up to 15 months before treatment initiation. AGV, CXM, and BSAP were measured during natural history study 111-901 and during the placebo-controlled double-blind phase 3 study 111-301. AGV was relatively stable before treatment initiation, and in subjects receiving placebo. In contrast, AGV dramatically increased in subjects receiving vosoritide by 3 months (FIG. 6). Similarly, CXM levels were dramatically increased in treated subjects in study 111-301, but not in subjects receiving placebo. Serum BSAP levels increased in treated subjects, but also increased to a lesser extent in subjects receiving placebo.

The data suggest that CXM is superior to CTxII, PINP, and BSAP for monitoring changes in endochondral bone growth. Data from the phase 3 study clearly demonstrated increased serum CXM levels that were associated with increased AGV in vosoritide-treated, but not placebo-treated subjects. There appeared to be some increase in BSAP in vosoritide-treated subjects over that observed in placebo-treated subjects, however this increase was relatively small compared with that observed for CXM. The study data demonstrated that serum CXM is a useful growth plate biomarker associated with changes in AGV.

### Example 3—High Throughput Characterization of NPR2 Variants Related to Genetic Causes of Short Stature

High throughput characterization of NPR2 variants will enable one of skill to better predict novel variants and, for those which occur more commonly, could improve diagnosis and clinical trial enrollment for eligible patients. It is hypothesized that the method herein is predictive of benign vs pathogenic classification of short stature gene variants, and NPR2 variant activity is predictive of overall height. Achondroplasia is defined as a height of <2SD from mean.

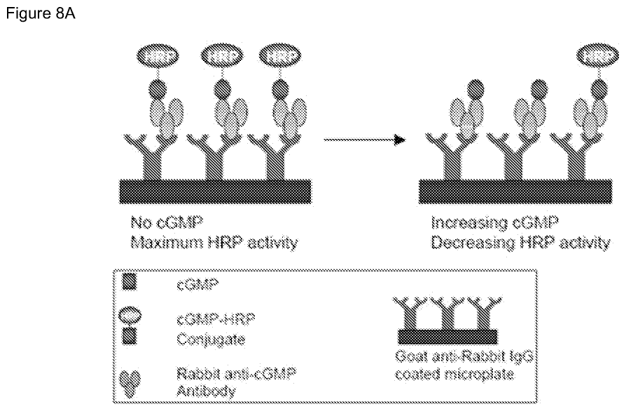

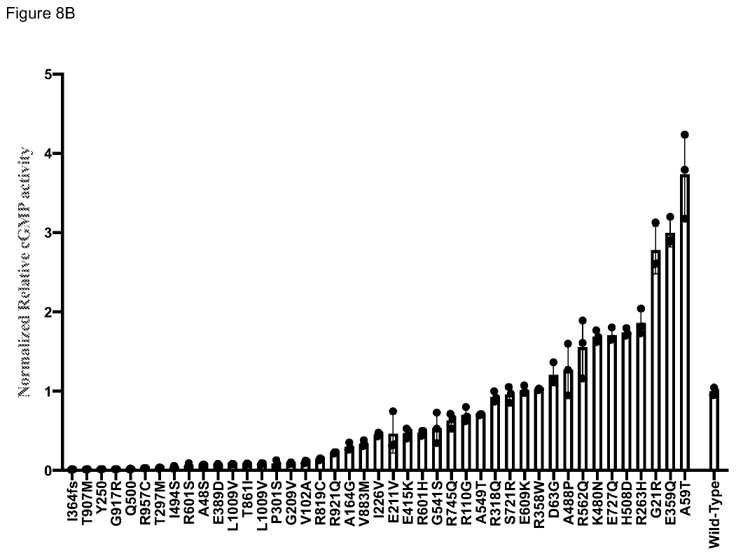

Described herein is an in-vitro assay (cGMP) to assess the activities of >260 NPR2 variants. NPR2 protein-altering variants have been identified in the UK Biobank study and described in Estrada et al. (Nat Commun. 2021 12 (1): 2224). NPR2-expression constructs were transfected into HEK293 cells and 3 days later cells were treated with 0.4 nM IBMX and 20 nM CNP in serum-free DMEM prior to measuring cGMP levels. The cGMP catchpoint ELISA competition assay (Molecular Devices) was used to measure cGMP according to manufacturer recommendations. RedLuc was used as a transfection control (FIG. 8A). FIG. 8B shows normalized cGMP values for a variety of LoF and GoF variants.

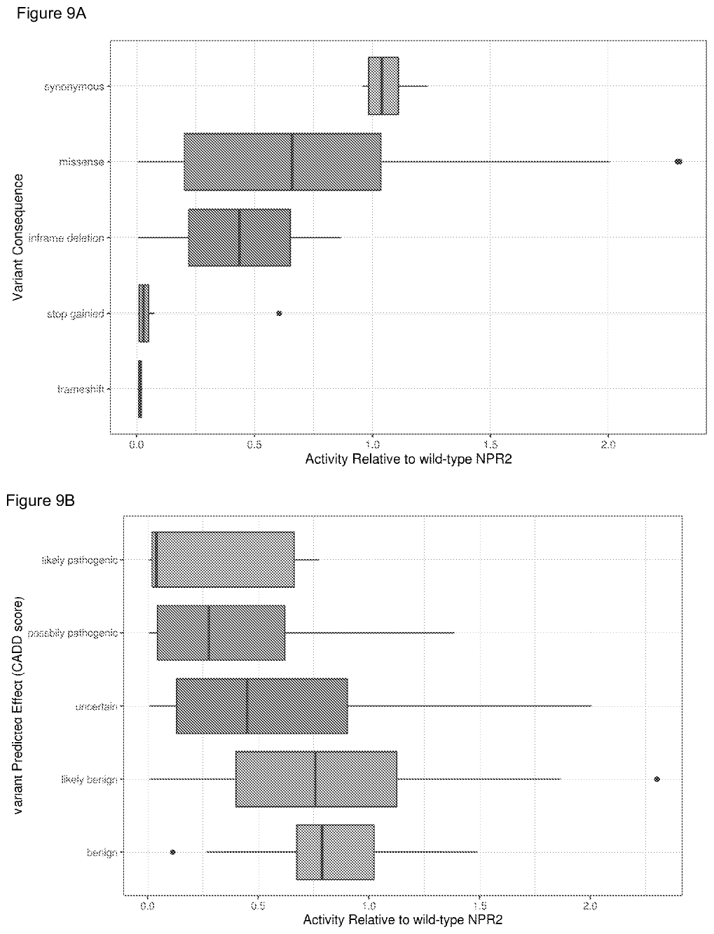

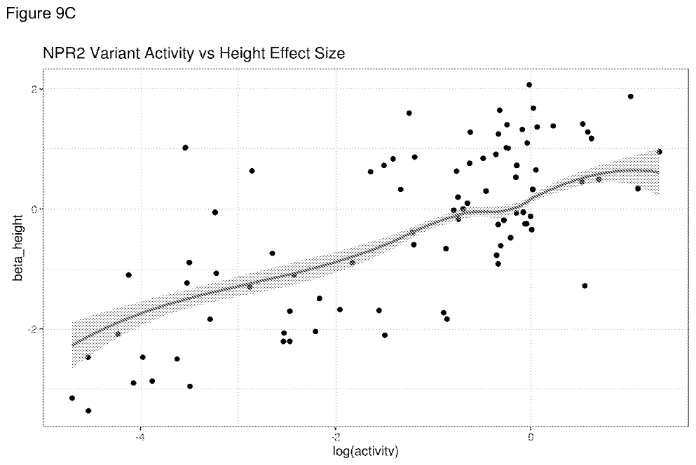

FIG. 9A shows a breakdown of variant activity level based on the predicted consequence for the protein. Protein truncating variants (stop gain and frameshifts) have activity levels near zero while synonymous mutations have activity levels near wild-type. Missense and in-frame deletions span a wide range of activity levels. FIG. 9B shows a breakdown of predicted consequences for missense variants based on Combined Annotation Dependent Depletion (CADD) scores (Kircher et al. A general framework for estimating the relative pathogenicity of human genetic variants. Nat Genet. 2014, 46 (3): 310-5), which incorporate evolutionary conservation and more than 60 other annotations. FIG. 9C is a comparison of the measured functional activities for NPR2 variants and the average impact on the height of individuals who carry them. These results show that the NPR2 variant activity data is predictive of height-effect size.

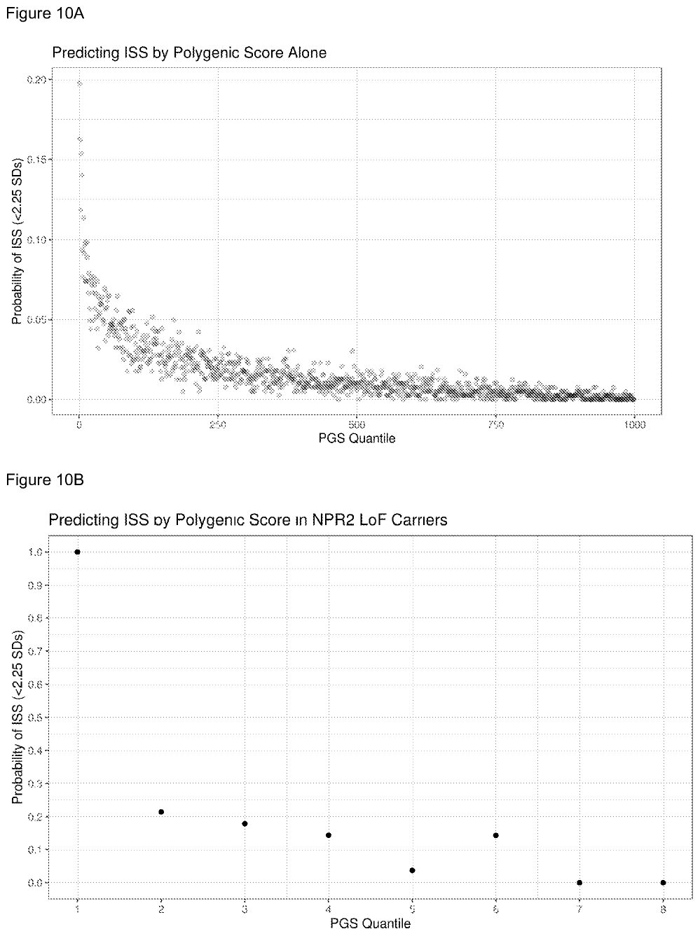

Polygenic Risk Score (PRS) can be used together with phenotypic data to identify patient populations. Idiopathic short stature (ISS) can be predicted based on genetics. FIG. 10A shows the probability of ISS based on polygenic scores for height alone. Polygenic scores summarize the combined effects of thousands of common variants with small effects on height. These scores capture 43% of the population variation in adult human height but have limited ability of predict at the extreme ends of the distribution. FIG. 10B shows how the predictive power of polygenic scores changes in the context of NPR2 loss of function variants. Individuals with an NPR2 LoF variant and a polygenic score in the bottom 12.5% have nearly 100% chance of ISS as an adult. Roughly 1 in 800 people have an NPR2 variant that has been characterized and confirmed low activity levels, so at least 1 in 6,600 could be predicted to have idiopathic short stature based on genetics alone. A similar procedure could be applied to other genes with large effects on height (ACAN, SHOX, DTL, PAPPA, etc.).

Mapping of select variants onto 3D structure reveals a potential mechanism of disruption. Approximately 160 NPR2 genetic variants were recently phenotypically characterized using a previously described cGMP-quantifying “catchpoint” assay (Estrada et al., supra). 3D modeling of high confidence loss-of-function (LoF) and Gain-of-function (GoF) variants could provide mechanistic insight into how the specific amino acid change and the position the variant could be modulating the activity of NPR2. The goal was to map ˜35 high-confidence (LoF and GoF) NPR2 variants onto known functional domains within NPR2 (2D mapping) and then map these variants onto the alpha-fold 3D structure (3D mapping).

The NPR2 alpha-fold structure was obtained from a recent alpha-fold publication (Jumper et al., Nature 596:583-589 (2021)). A list of phenotypically characterized “high-confidence” variants was generated (Table 4). Phenotype is calculated by determining levels of cGMP using a standard curve. This value to normalized to RedLuciferase (transfection control). This value is further normalized by setting WT to 1. Average value is calculated from at least 3 repeat experiments with 4 replicates each.

TABLE 4
Phenotypically characterized “high-confidence” variants

| # | Variant | average phenotype | Lable |
|---|---|---|---|
| 1 | Thr377Asn | 0.01061505 | LoF |
| 2 | Thr861Ile | 0.01124389 | LoF |
| 3 | Arg776Trp | 0.01312264 | LoF |
| 4 | Lys367Glu | 0.01438053 | LoF |
| 5 | Thr907Met* | 0.01456005 | LoF |
| 6 | Arg957Cys | 0.01552647 | LoF |
| 7 | Val965Asp | 0.01580358 | LoF |
| 8 | Gly917Arg* | 0.01708254 | LoF |
| 9 | Leu956Gln | 0.02434295 | LoF |
| 10 | Trp42Arg | 0.02880027 | LoF |
| 11 | Arg957Cys* | 0.0295365 | LoF |
| 12 | Thr297Met* | 0.03024853 | LoF |
| 13 | Ile494Ser* | 0.03638488 | LoF |
| 14 | Glu77Lys | 0.04985807 | LoF |
| 15 | Lys1006Thr | 0.05509553 | LoF |
| 16 | Ser522Pro | 0.05673193 | LoF |
| 17 | Arg601Ser* | 0.05948868 | LoF |
| 18 | Ala48Ser* | 0.06785073 | LoF |
| 19 | Glu389Asp* | 0.07079411 | LoF |
| 20 | Thr861Ile* | 0.07977669 | LoF |
| 21 | Leu1009Val* | 0.08469875 | LoF |
| 22 | Pro301Ser* | 0.08477935 | LoF |
| 23 | Gly209Val* | 0.08860852 | LoF |
| 24 | Lys629Arg | 1.49252348 | GoF |
| 25 | Val553Leu | 1.51545857 | GoF |
| 26 | Arg562Gln* | 1.55396206 | GoF |
| 27 | Arg562Gln | 1.62934316 | GoF |
| 28 | Lys480Asn* | 1.68888938 | GoF |
| 29 | Glu727Gln* | 1.70450503 | GoF |
| 30 | HIS508Asp* | 1.74002497 | GoF |
| 31 | His552Arg | 1.7937574 | GoF |
| 32 | Arg263His* | 1.86267462 | GoF |
| 33 | Gly21Arg* | 2.78149183 | GoF |
| 34 | Glu359Ala* | 2.99777785 | GoF |
| 35 | Ala59Thr | 3.73510437 | GoF |

*variants published in Estrada et al, 2021

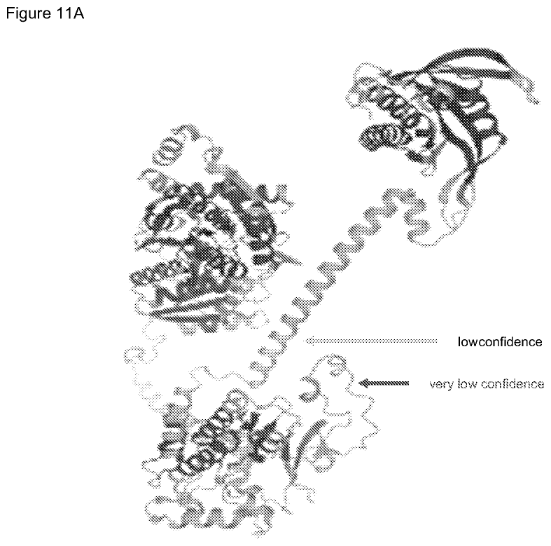

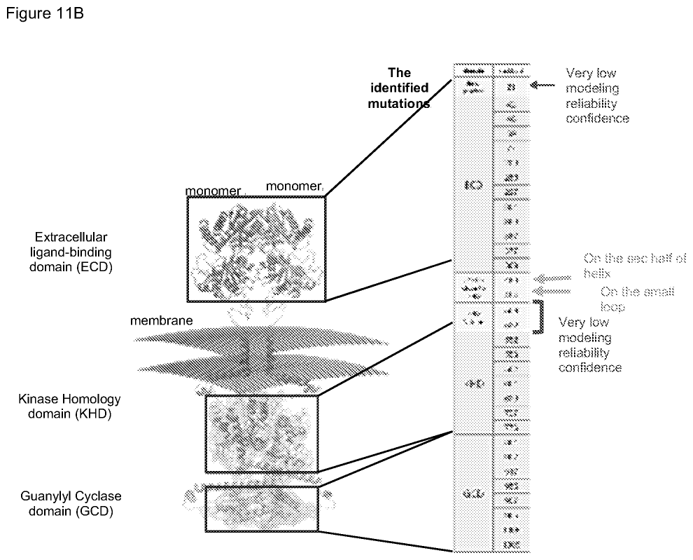

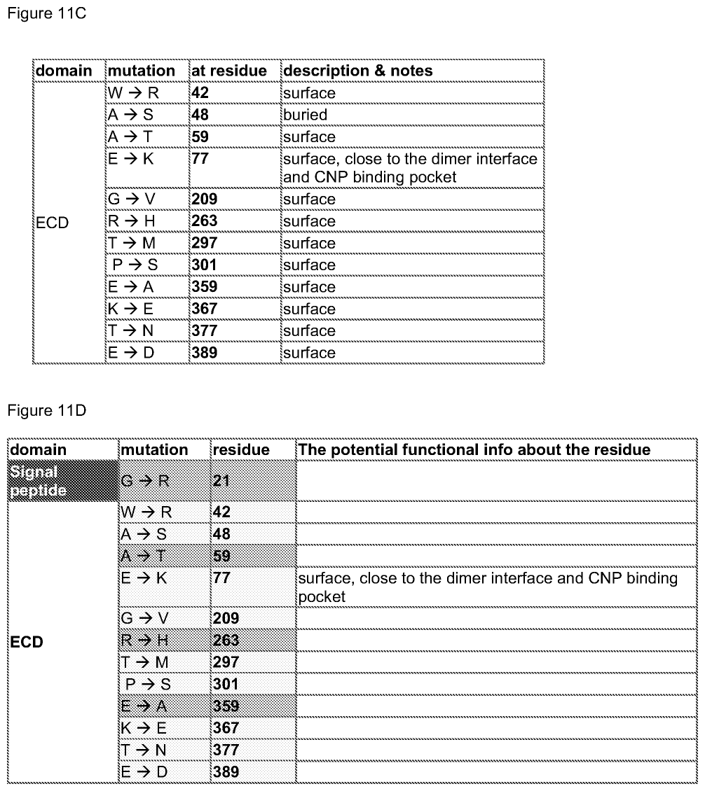

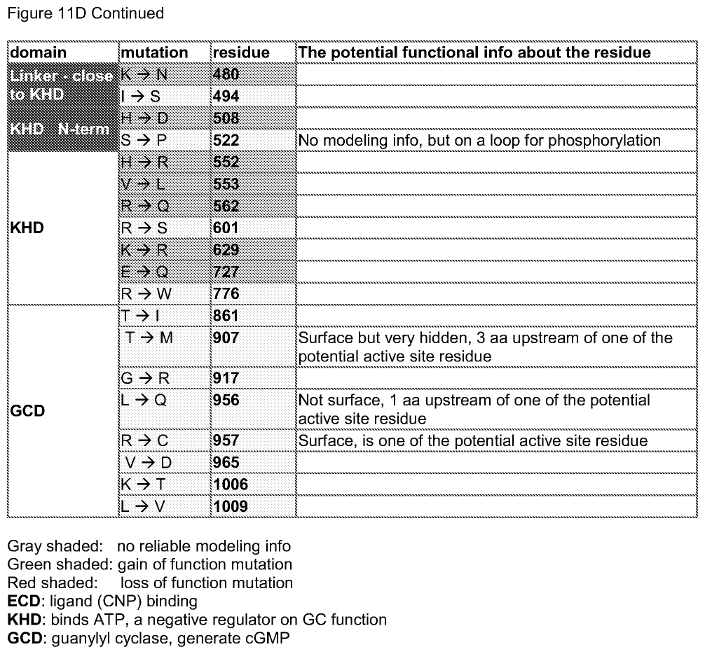

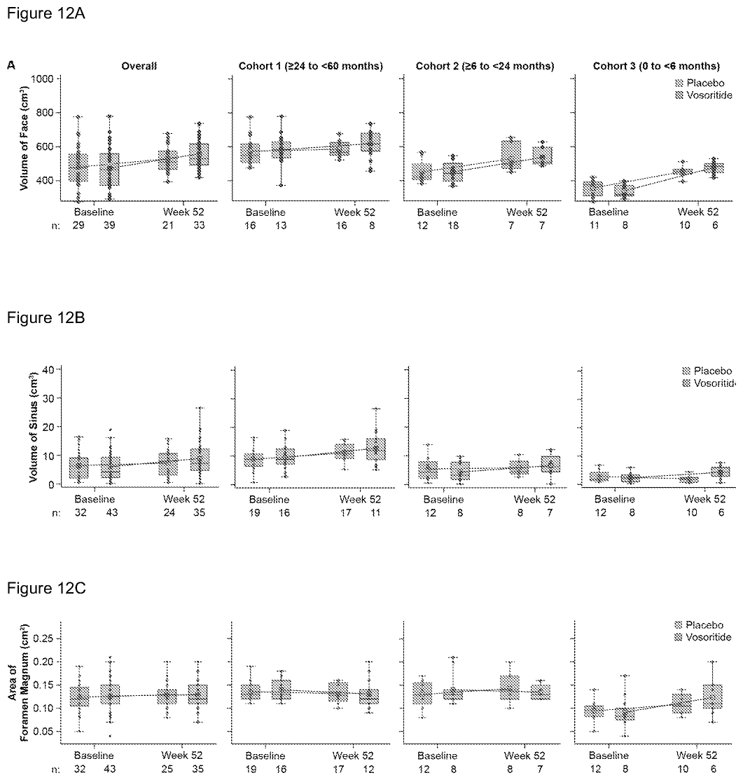

Variants were separated based on localization to different protein domains (ECD (extracellular domain): ligand (CNP) binding, KHD (kinase homology domain) (binds ATP, a negative regulator on GC function), or GCD (guanylyl cyclase domain: generates cGMP). These variants were mapped onto the alpha-fold 3D structure (FIG. 11A) and provide a conclusion as to the likely mechanism by which some variants could be altering NPR2 activity (FIG. 11C-11D). For example, many GoF mutations appear to be in the KHD region.

The data herein suggest that the presence of NPR LoF variant and a polygenic risk score (PRS) in the bottom 12.5% can accurately predict idiopathic short stature in adults. The present 3D modeling of high confidence LoF and GoF variants, provides mechanistic insight into these variant-specific effects of NPR2 activity.

### Example 4—Skull and Brain Morphology as a Measure of Efficacy of CNP Therapy

Additional markers for efficacy of CNP therapy include increase in skull and brain morphology, such as facial volume, sinus volume, and foramen magnum area.

In an ongoing Phase II study of CNP therapy in children, a group of patients age 3 to 6 months (cohort 3) were administered CNP (BMN111) at a dose of 30 μg/kg daily subcutaneously, and changes in annualized growth volume, changes in height, and skull morphology were measured. See also PCT Application No. PCT/US22/73605.

Pharmacokinetic studies showed participants in cohorts 2 and 3 who received 30 μg per kilogram of vosoritide had a higher average exposure than those in cohort 1 who received 15 μg per kilogram of vosoritide.

TABLE 5
Summary of plasma pharmacokinetic parameters of vosoritide for Study 206

| | | | | Median | | | |
|---|---|---|---|---|---|---|---|
| | Dose | Visit | | [range] Tₘₐₓ | Mean (SD) | Mean (SD) AUC₀₋ₜ | Mean (SD) |
| Cohort | (μg/kg) | (day) | No. | (min) | Cₘₐₓ (pg/mL) | (pg-min/mL) | t_{1/2} (min) |
| 1 | 15 | Day 1 | 18 | 15.0 [4.0, 30.0] | 4430 (3670) | 132,000 (97,300) | 21.7 (7.99) |
| | | Week 26 | 17 | 15.0 [6.0, 47.0] | 5850 (3260) | 282,000 (222,000) | 29.2 (7.06) |
| | | Week 52 | 16 | 15.0 [7.0, 31.0] | 5640 (2740) | 258,000 (169,000) | 29.0 (9.12) |
| 2 | 30 | Day 1 | 7 | 14.0 [4.0, 30.0] | 14,500 (5950) | 547,000 (279,000) | 33.3 (8.41) |
| | | Week 26 | 3 | 15.0 [6.0, 15.0] | 10,100 (2970) | 407,000 (148,000) | 21.0 (13.1) |
| | | Week 52 | 3 | 14.0 [13.0, 16.0] | 12,900 (6260) | 672,000 (33,4000) | 40.1 (15.2) |
| 3 | 30 | Day 1 | 11 | 5.0 [5.0, 14.0] | 13,400 (5710) | 323,000 (129,000) | 41.1 (18.8) |
| | | Week 26 | 6 | 6.5 [5.0, 15.0] | 11,500 (4290) | 297,000 (80,700) | 19.2 (6.14) |
| | | Week 52 | 8 | 15.5 [6.0, 17.0] | 13,500 (6770) | 557,000 (40,1000) | 24.3 (6.58) |

AUC, area under the plasma concentration-time curve from 0 to the time of last measurable concentration; SD, standard deviation; t_{1/2}, half life; Tₘₐₓ, peak time.

Magnetic Resonance Imaging (MRI) was used to ascertain possible treatment effects of vosoritide on brain and skull morphology, including foramen magnum, ventricular and brain parenchymal dimensions. It was also used to confirm that each patient going into the study is eligible based on the exclusion criteria (evidence of cervicomedullary stenosis, based on MRI of the brain obtained during the screening period or presence of a lesion or anatomical abnormality) indicating presence of clinically significant corticomedullary or spinal cord damage. Scan parameters were standardized across all clinical sites and are detailed in Table 6 below.

TABLE 6
MRI Parameters

| Parameter | Description |
|---|---|
| Superior extent | Vertex of skull (one slice above skull) |
| Inferior extent | Cervical C2 (one slice below C2) |
| Sequences | Scout |
| | Sagittal 3D T1-weighted image (T1WI) |
| | Sagittal 3D T2-weighted image (T2WI) |
| | Axial and coronal reconstructions of the sagittal |
| | T1 and T2 |
| Plane | Sagittal |
| Reconstruction | Axial and coronal |
| (MPR) plane | |
| Slice thickness | 1 mm (reconstructions performed with 1 mm) |
| Interslice gap | 0 mm (reconstructions performed with 0 mm) |
| (spacing) | |
| Matrix | 256 × 256 highly recommended |
| FOV | 20-25 cm required |
| NEX | 1-2 |
| Number of slices | All planes must have the same number of slices |
| | (to include reconstructions) |

T1-weighted and T2-weighted MRI studies (from the vertex of the skull to C2 at a slice thickness of 1.2 mm) were performed under anesthesia on trial subjects at baseline (screening period Day-30 to Day-1) and after 52 weeks of treatment (+/−7 days) or at the early termination visit using a standardized acquisition technique to ensure consistency over time and across sites.

In the youngest cohort (cohort 3, children age 3 to 6 months), participants treated with vosoritide versus placebo had greater increases in facial volume, facial sinus volume, and foramen magnum area at 52 weeks as assessed by MRI.

The MRI changes observed in participants in cohort 3 (age 3 to 6 months) are noteworthy as it is considered that abnormal craniofacial and base of skull endochondral ossification, leading to midfacial hypoplasia and stenosis of the foramen magnum, are major contributors to the excess incidence of sudden death reported in children with achondroplasia under age 5 years (Hecht et al., Am J Med Genet 1985; 20:355-60; Hoover-Fong et al., Bone 2021; 146:115872) (observed in 1 participant in this study) by causing sleep disordered breathing and brainstem compression (Hoover-Fong et al.). Given that vosoritide ameliorates the craniofacial skeletal and foramen magnum abnormalities in mouse models of achondroplasia (Lorget et al., Am J Hum Genet 2012; 91:1108-14) and accelerates endochondral ossification in children with achondroplasia (Savarirayan et al., The Lancet 2020; 396:684-92; Savarirayan et al., N Engl J Med 2019; 381:25-35), it is plausible that these MRI changes reflect a direct effect of vosoritide on craniofacial and foramen magnum growth. Whether these observed MRI changes translate into a decrease in the incidence of sudden infant death, sleep disordered breathing, and necessity for neurosurgical decompression of the foramen magnum in these infants will be assessed during the long-term follow-up study (Study 208, ClinicalTrials.gov number, NCT03989947).

The change in skull/brain morphology may be a better measure of efficacy in young patients since the treatment effect of vosoritide on annualized growth velocity in this group was not as high as in children 5 and older. Explanations for this discrepancy include the highly variable and rapidly declining growth velocity in very young children with achondroplasia, as well as the practical challenges in consistently and accurately measuring body length in these infants. The treatment effect observed on growth velocity in the youngest participants from Cohort 3 reflect this, with measurements showing wide variability and large confidence intervals.

Another ongoing study comparing current standard of care versus CNP variant (e.g., vosoritide) treatment in infants age less than 1 year with achondroplasia at risk of requiring surgical decompression of the foramen magnum will also directly address this issue (ClinicalTrials.gov number, NCT04554940) (Savarirayan et al., Sci Prog 2021; 104:368504211003782). These MRI changes were observed in participants in cohort 3 only, consistent with the fact that the growth of the foramen magnum, especially in the transverse plane, is negligible after age 6 months (Hecht et al., Am J Med Genet 1989; 32:528-35).

Once-daily subcutaneous administration of vosoritide in children aged 3 to 60 months was associated with an overall adverse-event profile that appears generally mild and resulted in increases in height Z-scores. Treatment in children aged 3 to 6 months resulted in increased facial and sinus volumes and increased area of the foramen magnum.

It is understood that every embodiment of the disclosure described herein may optionally be combined with any one or more of the other embodiments described herein. Every patent literature and every non-patent literature cited herein are incorporated herein by reference in their entirety.

It is understood, therefore, that this invention is not limited to the particular embodiments disclosed, but is intended to cover all modifications which are within the spirit and scope of the invention as defined by the appended claims; the above description, and/or shown in the attached drawings. Consequently only such limitations as appear in the appended claims should be placed on the disclosure.

What is claimed:

1. A method of treating a subject having a bone-related disorder, skeletal dysplasia or short stature and receiving C-type natriuretic peptide (CNP) therapy, comprising
i) administering CNP therapy to the subject;
ii) obtaining a sample from the subject;
iii) measuring levels of NTproCNP and/or N terminal fragment of collagen X (CXM) in a sample collected from the subject in (ii); and
iv) altering or changing the dose of CNP to bring NTproCNP levels within +/−2 SDS of mean NTproCNP for the population.

2. The method of claim 1, wherein CNP therapy dose level or frequency increases if the level of NTproCNP increases, or CNP therapy dose level decreases if the level of NTproCNP decreases.

3. A method of treating a subject having a bone-related disorder, skeletal dysplasia or short stature and receiving C-type natriuretic peptide (CNP) therapy, comprising
i) administering CNP therapy to the subject;
ii) obtaining a sample from the subject;
iii) measuring levels of N terminal fragment of collagen X (CXM) in a sample collected from the subject in (ii); and
iv) increasing CNP therapy dose level or frequency if the level of collagen X decreases.

4. The method of any one of claims 1-3, wherein increasing the CNP therapy dose increases the average growth velocity (AGV) in the subject.

5. The method of claim 4, wherein the average growth velocity (AGV) in the subject increases over 6 months, over 1 year or over 2 years, or more.

6. The method of any one of claims 1 to 5, wherein increasing CNP therapy dose comprises increasing dose frequency and/or increasing dose amount.

7. The method of any one of claims 1 to 6, wherein an increase in CNP therapy dose level and decrease in NTproCNP level correlate with improved Annualized Growth Velocity (AGV) in subjects.

8. The method of any one of claims 1 to 7, wherein an increase in CNP therapy dose level and decrease in NTproCNP level extends the duration of growth plate activity in the subject.

9. The method of any one of claims 1 to 8, wherein the levels of NTproCNP are maintained between +/−2 SDS of the mean NTproCNP for that population.

10. The method of any one of claims 1 to 8, wherein the CNP therapy is titrated toward zero NTproCNP SDS if the NTproCNP SDS is below the mean.

11. The method of claim 10, wherein the zero NTproCNP SDS predicts optimal effect size.

12. The method of any one of claims 1 to 11, wherein the sample is blood, urine, plasma, saliva, or tissue.

13. The method of any one of claims 1 to 12, wherein the subject is suffering from bone-related disorder, skeletal dysplasia or short stature selected from the group consisting of achondroplasia, osteoarthritis, hypophosphatemic rickets, hypochondroplasia, short stature, dwarfism, osteochondrodysplasias, thanatophoric dysplasia, osteogenesis imperfecta, achondrogenesis, chondrodysplasia punctata, homozygous achondroplasia, camptomelic dysplasia, congenital lethal hypophosphatasia, perinatal lethal type of osteogenesis imperfecta, short-rib polydactyly syndromes, rhizomelic type of chondrodysplasia punctata, Jansen-type metaphyseal dysplasia, spondyloepiphyseal dysplasia congenita, atelosteogenesis, diastrophic dysplasia, congenital short femur, Langer-type mesomelic dysplasia, Nievergelt-type mesomelic dysplasia, Robinow syndrome, Reinhardt syndrome, acrodysostosis, peripheral dysostosis, Kniest dysplasia, fibrochondrogenesis, Roberts syndrome, acromesomelic dysplasia, micromelia, Morquio syndrome, Kniest syndrome, metatrophic dysplasia, spondyloepimetaphyseal dysplasia, disorders related to NPR2 mutation, SHOX mutation (Turner's syndrome/Leri Weill), PTPN11 mutations (Noonan's syndrome) and IGF1R mutation.

14. The method of any one of claims 1 to 13, wherein the CNP is a CNP variant selected from the group consisting of
(Pro-Gly-CNP37)
(SEQ ID NO: 1)
PGQEHPNARKYKGANKKGLSKGCFGLKLDRIGSMSGLGC;
(Gly-CNP-37)
(SEQ ID NO: 2)
GQEHPNARKYKGANKKGLSKGCFGLKLDRIGSMSGLGC;
(Gly-CNP53)
(SEQ ID NO: 3)
GDLRVDTKSRAAWARLLQEHPNARKYKGANKKGLSKGCFGLKLDRIGSM
SGLGC;
(Pro-CNP53)
(SEQ ID NO: 4)
PDLRVDTKSRAAWARLLQEHPNARKYKGANKKGLSKGCFGLKLDRIGSM
SGLGC;
(Met-CNP53)
(SEQ ID NO: 5)
MDLRVDTKSRAAWARLLQEHPNARKYKGANKKGLSKGCFGLKLDRIGSM
SGLGC;
[CNP-53(M48N)]
(SEQ ID NO: 6)
DLRVDTKSRAAWARLLQEHPNARKYKGANKKGLSKGCFGLKLDRIGSNS
GLGC;
(CNP-52)
(SEQ ID NO: 7)
LRVDTKSRAAWARLLQEHPNARKYKGANKKGLSKGCFGLKLDRIGSMSG
LGC;
(CNP-51)
(SEQ ID NO: 8)
RVDTKSRAAWARLLQEHPNARKYKGANKKGLSKGCFGLKLDRIGSMSGL
GC;
(CNP-50)
(SEQ ID NO: 9)
VDTKSRAAWARLLQEHPNARKYKGANKKGLSKGCFGLKLDRIGSMSGLG
C;
(CNP-49)
(SEQ ID NO: 10)
DTKSRAAWARLLQEHPNARKYKGANKKGLSKGCFGLKLDRIGSMSGLG
C;
(CNP-48)
(SEQ ID NO: 11)
TKSRAAWARLLQEHPNARKYKGANKKGLSKGCFGLKLDRIGSMSGLGC;
(CNP-47)
(SEQ ID NO: 12)
KSRAAWARLLQEHPNARKYKGANKKGLSKGCFGLKLDRIGSMSGLGC;
(CNP-46)
(SEQ ID NO: 13)
SRAAWARLLQEHPNARKYKGANKKGLSKGCFGLKLDRIGSMSGLGC;
(CNP-45)
(SEQ ID NO: 14)
RAAWARLLQEHPNARKYKGANKKGLSKGCFGLKLDRIGSMSGLGC;
(CNP-44)
(SEQ ID NO: 15)
AAWARLLQEHPNARKYKGANKKGLSKGCFGLKLDRIGSMSGLGC;
(CNP-43)
(SEQ ID NO: 16)
AWARLLQEHPNARKYKGANKKGLSKGCFGLKLDRIGSMSGLGC;
(CNP-42)
(SEQ ID NO: 17)
WARLLQEHPNARKYKGANKKGLSKGCFGLKLDRIGSMSGLGC;
(CNP-41)
(SEQ ID NO: 18)
ARLLQEHPNARKYKGANKKGLSKGCFGLKLDRIGSMSGLGC;
(CNP-40)
(SEQ ID NO: 19)
RLLQEHPNARKYKGANKKGLSKGCFGLKLDRIGSMSGLGC;
(CNP-39)
(SEQ ID NO: 20)
LLQEHPNARKYKGANKKGLSKGCFGLKLDRIGSMSGLGC;
(CNP-38)
(SEQ ID NO: 21)
LQEHPNARKYKGANKKGLSKGCFGLKLDRIGSMSGLGC;
(CNP-37)
(SEQ ID NO: 22)
QEHPNARKYKGANKKGLSKGCFGLKLDRIGSMSGLGC;
(CNP-36)
(SEQ ID NO: 23)
EHPNARKYKGANKKGLSKGCFGLKLDRIGSMSGLGC;
(CNP-35)
(SEQ ID NO: 24)
HPNARKYKGANKKGLSKGCFGLKLDRIGSMSGLGC;
(CNP-34)
(SEQ ID NO: 25)
PNARKYKGANKKGLSKGCFGLKLDRIGSMSGLGC;
(CNP-33)
(SEQ ID NO: 26)
NARKYKGANKKGLSKGCFGLKLDRIGSMSGLGC;
(CNP-32)
(SEQ ID NO: 27)
ARKYKGANKKGLSKGCFGLKLDRIGSMSGLGC;
(CNP-31)
(SEQ ID NO: 28)
RKYKGANKKGLSKGCFGLKLDRIGSMSGLGC;
(CNP-30)
(SEQ ID NO: 29)
KYKGANKKGLSKGCFGLKLDRIGSMSGLGC;
(CNP-29)
(SEQ ID NO: 30)
YKGANKKGLSKGCFGLKLDRIGSMSGLGC;
(CNP-28)
(SEQ ID NO: 31)
KGANKKGLSKGCFGLKLDRIGSMSGLGC;
(CNP-27)
(SEQ ID NO: 32)
GANKKGLSKGCFGLKLDRIGSMSGLGC;
(CNP-26)
(SEQ ID NO: 33)
ANKKGLSKGCFGLKLDRIGSMSGLGC;
(CNP-25)
(SEQ ID NO: 34)
NKKGLSKGCFGLKLDRIGSMSGLGC;
(CNP-24)
(SEQ ID NO: 35)
KKGLSKGCFGLKLDRIGSMSGLGC;
(CNP-23)
(SEQ ID NO: 36)
KGLSKGCFGLKLDRIGSMSGLGC;
(CNP-21)
(SEQ ID NO: 37)
LSKGCFGLKLDRIGSMSGLGC;
(CNP-20)
(SEQ ID NO: 38)
SKGCFGLKLDRIGSMSGLGC;
(CNP-19)
(SEQ ID NO: 39)
KGCFGLKLDRIGSMSGLGC;
(CNP-18)
(SEQ ID NO: 40)
GCFGLKLDRIGSMSGLGC;
[CNP-37(M32N)]
(SEQ ID NO: 41)
QEHPNARKYKGANKKGLSKGCFGLKLDRIGSNSGLGC;
(Pro-CNP-37)
(SEQ ID NO: 42)
PQEHPNARKYKGANKKGLSKGCFGLKLDRIGSMSGLGC;
(Met-CNP-37)
(SEQ ID NO: 43)
MQEHPNARKYKGANKKGLSKGCFGLKLDRIGSMSGLGC;
[Gly-CNP-37(M32N)]
(SEQ ID NO: 44)
GQEHPNARKYKGANKKGLSKGCFGLKLDRIGSNSGLGC;
(Met-Gly-CNP-37)
(SEQ ID NO: 45)
MGQEHPNARKYKGANKKGLSKGCFGLKLDRIGSMSGLGC;
(SEQ ID NO: 46)
PGQEHPQARRYRGAQRRGLSRGCFGLKLDRIGSMSGLGC;
(SEQ ID NO: 47)
PGQEHPNARKYKGANKKGLSKGCFGLKLDRIGSMSGLGC;
(SEQ ID NO: 48)
PGQEHPNARRYRGANRRGLSRGCFGLKLDRIGSMSGLGC;
and
(SEQ ID NO: 49)
PGQEHPQARKYKGAQKKGLSKGCFGLKLDRIGSMSGLGC.

15. The method of claim 14, wherein the CNP variant is
(Pro-Gly-CNP37)
(SEQ ID NO: 1)
PGQEHPNARKYKGANKKGLSKGCFGLKLDRIGSMSGLGC;
(Gly-CNP-37)
(SEQ ID NO: 2)
GQEHPNARKYKGANKKGLSKGCFGLKLDRIGSMSGLGC;
or
(CNP-38)
(SEQ ID NO: 21)
LQEHPNARKYKGANKKGLSKGCFGLKLDRIGSMSGLGC.

16. The method of any one of claims 1 to 15, wherein levels of NTproCNP or CXM are measured in a plasma sample.

17. The method of any one of claims 1 to 16, wherein the subject is receiving between 7.5 μg/kg and 30 μg/kg CNP therapy.

18. The method of any one of claims 1 to 17, wherein the dose of CNP is increased to 30 μg/kg.

19. The method of any one of claims 1 to 18, wherein the NTproCNP and/or CXM is measured at least 4 hours after administration.

20. The method of any one of claims 1 to 19, wherein the level of NTproCNP and/or CXM is measured at least 6 months after start of CNP therapy.

21. The method of any one of claims 1 to 20, wherein the level of NTproCNP in a sample is compared to a baseline measurement taken prior to start of CNP therapy.

22. The method of any one of claims 1 to 21, wherein CNP therapy dose or frequency is increased when a decrease in NTproCNP indicates an increase in AGV in the subject.

23. The method of any one of claims 1 to 18, wherein the level of CXM in a sample is compared to a baseline measurement taken prior to start of CNP therapy.

24. The method of any one of claim 1-5, 12-20, or 23, wherein the CXM increase indicates increased bone growth, and wherein the dose of CNP frequency or level is increased when there is CXM increase that enhances AGV.

25. The method of any one of claims 1 to 24, wherein the subject is a pediatric subject with open growth plates and received a dose of 15 or 30 μg/kg daily.

26. A method of selecting initiation of CNP therapy in a subject comprising
i) measuring NTproCNP in the subject at multiple timepoints to establish a baseline NTproCNP level;
ii) determining if the NTproCNP levels indicate an SDS of zero, below or above zero; and
iii) starting treatment with CNP therapy when the subject has NTproCNP levels +/−2 SDS.

27. A method of selecting initiation of CNP therapy in a subject having achondroplasia comprising
i) measuring NTproCNP in the subject at multiple timepoints to establish a baseline NTproCNP level;
ii) determining if the NTproCNP levels indicate an SDS of zero or above zero; and
iii) starting treatment with CNP therapy when the subject has NTproCNP levels above SDS zero.

28. The method of claim 27, wherein NTproCNP is measured at 2 weeks, one month, 3 months, and 6 months to establish a baseline NTproCNP level.

29. The method of any one of claims 1 to 28, wherein NTproCNP is measured by radioimmunoassay.

30. A method of treating a subject having a bone-related disorder, skeletal dysplasia or short stature comprising,
[i) identifying whether a subject has a Loss of Function (LoF) or Gain of Function (GoF) variant of a gene related to a bone-related disorder, skeletal dysplasia or short stature;
ii) calculating a polygenic risk score (PRS) for height of the subject;
iii) determining if the subject has a LoF variant and a PRS in the bottom 20%; and
iv) treating the subject with a CNP variant if the subject has a LoF variant and a PRS in the bottom 20%.

31. The method of claim 30, wherein the gene related to a bone-related disorder, skeletal dysplasia or short stature is selected from the group consisting of NPR2, SHOX, PTPN11, COL2A1, COL11A1, COL9A2, COL10), aggrecan (ACAN), indian hedgehog (IHH), NPPC, FGFR3, IGF1R, DTL, and pregnancy-associated plasma protein A2 (PAPPA2) or combinations thereof.

32. The method of claim 30 or 31, wherein the gene related to a bone-related disorder, skeletal dysplasia or short stature is NPR2.

33. A method for increasing facial volume, facial sinus volume, and foramen magnum area in a subject 6 months old or less having a bone-related disorder, skeletal dysplasia or short stature comprising administering a CNP variant at a dose of at least 30 μg/kg.

34. A method of decreasing the incidence of sudden infant death, sleep disordered breathing, and neurosurgical decompression of the foramen magnum in a subject 6 months old or less having a bone-related disorder, skeletal dysplasia or short stature comprising administering CNP variant at a dose of at least 30 μg/kg.

35. The method of claim 33 or 34 wherein the increase in facial volume, facial sinus volume, and foramen magnum area are measured by magnetic resonance imaging (MRI).

36. The method of claim 35, wherein the change in facial volume, facial sinus volume, and foramen magnum area are compared to baseline levels, healthy control subjects or untreated control subjects.

37. The method of any one of claims 33 to 36, wherein the CNP variant is administered subcutaneously.

38. The method of any one of claims 33 to 37, wherein CNP variant is administered daily, weekly, every 2 weeks, monthly, or less.

39. The method of any one of claims 33 to 38, wherein the CNP variant is administered at a dose of 30 μg/kg for 3 months, 6 months, 1 year or more.

40. The method of any one of claims 33 to 39, wherein the dose of CNP variant is decreased to 15 μg/kg when the subject is about 2 years old.


SEQUENCE LISTING (53 sequences)

SEQ ID NO: 1 - protein, 39 aa, synthetic construct
  source 1..39: note = Pro-Gly-CNP37
PGQEHPNARKYKGANKKGLSKGCFGLKLDRIGSMSGLGC

SEQ ID NO: 2 - protein, 38 aa, synthetic construct
  source 1..38: note = Gly-CNP37
GQEHPNARKYKGANKKGLSKGCFGLKLDRIGSMSGLGC

SEQ ID NO: 3 - protein, 54 aa, synthetic construct
  source 1..54: note = Gly-CNP53
GDLRVDTKSRAAWARLLQEHPNARKYKGANKKGLSKGCFGLKLDRIGSMSGLGC

SEQ ID NO: 4 - protein, 54 aa, synthetic construct
  source 1..54: note = Pro-CNP53
PDLRVDTKSRAAWARLLQEHPNARKYKGANKKGLSKGCFGLKLDRIGSMSGLGC

SEQ ID NO: 5 - protein, 54 aa, synthetic construct
  source 1..54: note = Met-CNP53
MDLRVDTKSRAAWARLLQEHPNARKYKGANKKGLSKGCFGLKLDRIGSMSGLGC

SEQ ID NO: 6 - protein, 53 aa, synthetic construct
  source 1..53: note = CNP-53(M48N)
DLRVDTKSRAAWARLLQEHPNARKYKGANKKGLSKGCFGLKLDRIGSNSGLGC

SEQ ID NO: 7 - protein, 52 aa, synthetic construct
  source 1..52: note = CNP-52
LRVDTKSRAAWARLLQEHPNARKYKGANKKGLSKGCFGLKLDRIGSMSGLGC

SEQ ID NO: 8 - protein, 51 aa, synthetic construct
  source 1..51: note = CNP-51
RVDTKSRAAWARLLQEHPNARKYKGANKKGLSKGCFGLKLDRIGSMSGLGC

SEQ ID NO: 9 - protein, 50 aa, synthetic construct
  source 1..50: note = CNP-50
VDTKSRAAWARLLQEHPNARKYKGANKKGLSKGCFGLKLDRIGSMSGLGC

SEQ ID NO: 10 - protein, 49 aa, synthetic construct
  source 1..49: note = CNP-49
DTKSRAAWARLLQEHPNARKYKGANKKGLSKGCFGLKLDRIGSMSGLGC

SEQ ID NO: 11 - protein, 48 aa, synthetic construct
  source 1..48: note = CNP-48
TKSRAAWARLLQEHPNARKYKGANKKGLSKGCFGLKLDRIGSMSGLGC

SEQ ID NO: 12 - protein, 47 aa, synthetic construct
  source 1..47: note = CNP-47
KSRAAWARLLQEHPNARKYKGANKKGLSKGCFGLKLDRIGSMSGLGC

SEQ ID NO: 13 - protein, 46 aa, synthetic construct
  source 1..46: note = CNP-46
SRAAWARLLQEHPNARKYKGANKKGLSKGCFGLKLDRIGSMSGLGC

SEQ ID NO: 14 - protein, 45 aa, synthetic construct
  source 1..45: note = CNP-45
RAAWARLLQEHPNARKYKGANKKGLSKGCFGLKLDRIGSMSGLGC

SEQ ID NO: 15 - protein, 44 aa, synthetic construct
  source 1..44: note = CNP-44
AAWARLLQEHPNARKYKGANKKGLSKGCFGLKLDRIGSMSGLGC

SEQ ID NO: 16 - protein, 43 aa, synthetic construct
  source 1..43: note = CNP-43
AWARLLQEHPNARKYKGANKKGLSKGCFGLKLDRIGSMSGLGC

SEQ ID NO: 17 - protein, 42 aa, synthetic construct
  source 1..42: note = CNP-42
WARLLQEHPNARKYKGANKKGLSKGCFGLKLDRIGSMSGLGC

SEQ ID NO: 18 - protein, 41 aa, synthetic construct
  source 1..41: note = CNP-41
ARLLQEHPNARKYKGANKKGLSKGCFGLKLDRIGSMSGLGC

SEQ ID NO: 19 - protein, 40 aa, synthetic construct
  source 1..40: note = CNP-40
RLLQEHPNARKYKGANKKGLSKGCFGLKLDRIGSMSGLGC

SEQ ID NO: 20 - protein, 39 aa, synthetic construct
  source 1..39: note = CNP-39
LLQEHPNARKYKGANKKGLSKGCFGLKLDRIGSMSGLGC

SEQ ID NO: 21 - protein, 38 aa, synthetic construct
  source 1..38: note = CNP-38
LQEHPNARKYKGANKKGLSKGCFGLKLDRIGSMSGLGC

SEQ ID NO: 22 - protein, 37 aa, synthetic construct
  source 1..37: note = CNP-37
QEHPNARKYKGANKKGLSKGCFGLKLDRIGSMSGLGC

SEQ ID NO: 23 - protein, 36 aa, synthetic construct
  source 1..36: note = CNP-36
EHPNARKYKGANKKGLSKGCFGLKLDRIGSMSGLGC

SEQ ID NO: 24 - protein, 35 aa, synthetic construct
  source 1..35: note = CNP-35
HPNARKYKGANKKGLSKGCFGLKLDRIGSMSGLGC

SEQ ID NO: 25 - protein, 34 aa, synthetic construct
  source 1..34: note = CNP-34
PNARKYKGANKKGLSKGCFGLKLDRIGSMSGLGC

SEQ ID NO: 26 - protein, 33 aa, synthetic construct
  source 1..33: note = CNP-33
NARKYKGANKKGLSKGCFGLKLDRIGSMSGLGC

SEQ ID NO: 27 - protein, 32 aa, synthetic construct
  source 1..32: note = CNP-32
ARKYKGANKKGLSKGCFGLKLDRIGSMSGLGC

SEQ ID NO: 28 - protein, 31 aa, synthetic construct
  source 1..31: note = CNP-31
RKYKGANKKGLSKGCFGLKLDRIGSMSGLGC

SEQ ID NO: 29 - protein, 30 aa, synthetic construct
  source 1..30: note = CNP-30
KYKGANKKGLSKGCFGLKLDRIGSMSGLGC

SEQ ID NO: 30 - protein, 29 aa, synthetic construct
  source 1..29: note = CNP-29
YKGANKKGLSKGCFGLKLDRIGSMSGLGC

SEQ ID NO: 31 - protein, 28 aa, synthetic construct
  source 1..28: note = CNP-28
KGANKKGLSKGCFGLKLDRIGSMSGLGC

SEQ ID NO: 32 - protein, 27 aa, synthetic construct
  source 1..27: note = CNP-27
GANKKGLSKGCFGLKLDRIGSMSGLGC

SEQ ID NO: 33 - protein, 26 aa, synthetic construct
  source 1..26: note = CNP-26
ANKKGLSKGCFGLKLDRIGSMSGLGC

SEQ ID NO: 34 - protein, 25 aa, synthetic construct
  source 1..25: note = CNP-25
NKKGLSKGCFGLKLDRIGSMSGLGC

SEQ ID NO: 35 - protein, 24 aa, synthetic construct
  source 1..24: note = CNP-24
KKGLSKGCFGLKLDRIGSMSGLGC

SEQ ID NO: 36 - protein, 23 aa, synthetic construct
  source 1..23: note = CNP-23
KGLSKGCFGLKLDRIGSMSGLGC

SEQ ID NO: 37 - protein, 21 aa, synthetic construct
  source 1..21: note = CNP-21
LSKGCFGLKLDRIGSMSGLGC

SEQ ID NO: 38 - protein, 20 aa, synthetic construct
  source 1..20: note = CNP-20
SKGCFGLKLDRIGSMSGLGC

SEQ ID NO: 39 - protein, 19 aa, synthetic construct
  source 1..19: note = CNP-19
KGCFGLKLDRIGSMSGLGC

SEQ ID NO: 40 - protein, 18 aa, synthetic construct
  source 1..18: note = CNP-18
GCFGLKLDRIGSMSGLGC

SEQ ID NO: 41 - protein, 37 aa, synthetic construct
  source 1..37: note = CNP37(M32N)
QEHPNARKYKGANKKGLSKGCFGLKLDRIGSNSGLGC

SEQ ID NO: 42 - protein, 38 aa, synthetic construct
  source 1..38: note = Pro-CNP37
PQEHPNARKYKGANKKGLSKGCFGLKLDRIGSMSGLGC

SEQ ID NO: 43 - protein, 38 aa, synthetic construct
  source 1..38: note = Met-CNP37
MQEHPNARKYKGANKKGLSKGCFGLKLDRIGSMSGLGC

SEQ ID NO: 44 - protein, 38 aa, synthetic construct
  source 1..38: note = Gly-CNP37(M32N)
GQEHPNARKYKGANKKGLSKGCFGLKLDRIGSNSGLGC

SEQ ID NO: 45 - protein, 39 aa, synthetic construct
  source 1..39: note = Met-Gly-CNP37
MGQEHPNARKYKGANKKGLSKGCFGLKLDRIGSMSGLGC

SEQ ID NO: 46 - protein, 39 aa, synthetic construct
PGQEHPQARRYRGAQRRGLSRGCFGLKLDRIGSMSGLGC

SEQ ID NO: 47 - protein, 39 aa, synthetic construct; identical to SEQ ID NO: 1

SEQ ID NO: 48 - protein, 39 aa, synthetic construct
PGQEHPNARRYRGANRRGLSRGCFGLKLDRIGSMSGLGC

SEQ ID NO: 49 - protein, 39 aa, synthetic construct
PGQEHPQARKYKGAQKKGLSKGCFGLKLDRIGSMSGLGC

SEQ ID NO: 50 - protein, 38 aa, synthetic construct
GQEHPNARKYKGANPKGLSKGCFGLKLDRIGSMSGLGC

SEQ ID NO: 51 - protein, 38 aa, synthetic construct
GQEHPNARKYKGANQKGLSKGCFGLKLDRIGSMSGLGC

SEQ ID NO: 52 - protein, 38 aa, synthetic construct
GQEHPNARKYKGANQQGLSKGCFGLKLDRIGSMSGLGC

SEQ ID NO: 53 - protein, 38 aa, synthetic construct
GQEHPNARKYKGANKPGLSKGCFGLKLDRIGSMSGLGC